(19) United States
(12) Patent Application Publication
(10) Pub. No.: US 2026/0176358 A1
(43) Pub. Date: Jun. 25, 2026

(54) ANTIGEN-BINDING MOLECULES THAT SPECIFICALLY BIND CGRP AND PACAP AND PHARMACEUTICAL USE THEREOF

(71) Applicants: Jiangsu Hengrui Pharmaceuticals Co., Ltd., Lianyungang, Jiangsu (CN); Shanghai Hengrui Pharmaceutical Co., Ltd., Shanghai (CN)

(72) Inventors: Qian Wang, Shanghai (CN); Manman Zhu, Shanghai (CN); Xinsheng Jin, Shanghai (CN); Jinping Shi, Shanghai (CN); Hua Ying, Shanghai (CN); Weikang Tao, Shanghai (CN)

(21) Appl. No.: 19/124,870

(22) PCT Filed: Sep. 30, 2022
(86) PCT No.: PCT/CN2022/123272

§ 371 Date: Apr. 27, 2025

(30) Foreign Application Priority Data
Sep. 30, 2021 (CN) 202111157522.9

(51) Int. Cl. C07K 16/28; C07K 16/00; C07K 16/22

(52) U.S. Cl. CPC C07K 16/2809; C07K 16/00; C07K 16/22; C07K 2317/56; C07K 2317/565; C07K 2317/76

(57) ABSTRACT

The present disclosure relates to antigen-binding molecules that specifically bind CORP and PACAP and a pharmaceutical use thereof. The antigen-binding molecules can be used to treat pain-related diseases.

### CROSS-REFERENCE TO RELATED APPLICATIONS

The present application is a U.S. National Phase of International PCT Application No. PCT/CN2022/123272 filed on Sep. 30, 2022, which claims priority to Chinese Patent Application No. 202111157522.9 filed on Sep. 30, 2021, the contents of each application are incorporated herein by reference in their entireties.

### SEQUENCE LISTING

This application incorporates by reference the material in the ST.26 XML file titled CHENG-81_722120CPUS_Sequence-listing, which was created on Apr. 28, 2025 and is 256,120 bytes.

### TECHNICAL FIELD

The present disclosure pertains to the field of biotechnology and particularly relates to an antigen-binding molecule and use thereof.

### BACKGROUND

The statements herein merely provide background information related to the present disclosure and may not necessarily constitute the prior art.

CGRP (calcitonin gene-related peptide) is an effective vasodilatory neuropeptide consisting of 37 amino acids. CGRPs of different species are highly homologous. The CGRP receptor consists of two components: RAMP1, a single-transmembrane component, and CALCRL, a seven-transmembrane component. During migraine attacks, the binding of CGRP to its receptor activates adenylate cyclase, thereby elevating cAMP levels, leading to vasodilation, and causing pain. Anti-pain drugs can be developed by inhibiting the release of CGRP or preventing the binding of CGRP to its receptor. Currently commercially available CGRP-related antibodies all demonstrate a clinical response rate of only 50%. PACAP (pituitary adenylate cyclase-activating polypeptide) exists in two forms: 90% of PACAP molecules are in the form of 38 amino acids (PACAP 38), and 10% in the form of 27 amino acids (PACAP 27). PACAP 27 is a spliceosomal form of the first 27 amino acids of PACAP 38. The PACAP sequences of all species are identical. PACAP and vasoactive intestinal peptide (VIP) have 68% sequence homology and share three receptors. PACAP and VIP have the same affinity for the receptors VPAC1 and VPAC2; however, the affinity of PACAP for the receptor PAC1 is 1000 times higher than that of VIP. PACAP injections can induce migraine attacks, while VIP injections cannot. No PACAP-related antibodies are currently commercially available for treating headaches.

Therefore, there is an unmet need for more active drugs for treating pain.

### SUMMARY

The present disclosure provides an antigen-binding molecule that specifically binds to CGRP and PACAP. These antigen-binding molecules are capable of providing better therapeutic activity than anti-CGRP antibodies and anti-PACAP antibodies.

In one aspect, the present disclosure provides an antigen-binding molecule comprising at least one antigen-binding moiety that specifically binds to CGRP and at least one antigen-binding moiety that specifically binds to PACAP, wherein the antigen-binding moiety that specifically binds to CGRP comprises a heavy chain variable region (CGRP-VH) and a light chain variable region (CGRP-VL), and the antigen-binding moiety that specifically binds to PACAP comprises a heavy chain variable region (PACAP-VH) and a light chain variable region (PACAP-VL).

In some embodiments, the antigen-binding molecule binds to human CGRP at 25° C. with a KD of less than 1×10⁻¹⁰ M, as measured by a surface plasmon resonance assay.

In some embodiments, the antigen-binding molecule binds to rat CGRP at 25° C. with a KD of less than 1×10⁻⁹ M, as measured by a surface plasmon resonance assay.

In some embodiments, the antigen-binding molecule binds to human CGRP or rat CGRP with an EC₅₀ of less than 1×10⁻⁸ M, as measured by an ELISA.

In some embodiments, the antigen-binding molecule binds to PACAP38 and PACAP27 with an EC₅₀ of less than 1×10⁻⁹ M, as measured by an ELISA.

In some embodiments, the antigen-binding molecule does not bind to VIP. In some embodiments, the antigen-binding molecule inhibits cAMP production in cells with an IC₅₀ of less than 4×10⁻⁸ M under conditions of CGRP induction; and/or in some embodiments, the antigen-binding molecule inhibits cAMP production in cells with an IC₅₀ of less than 2×10⁻⁸ M under conditions of PACAP induction.

In some embodiments, provided is the antigen-binding molecule according to any one of the foregoing, wherein:
(i) a CGRP-HCDR1, a CGRP-HCDR2, and a CGRP-HCDR3 in the CGRP-VH comprise the amino acid sequences of a CGRP-HCDR1, a CGRP-HCDR2, and a CGRP-HCDR3 in SEQ ID NO: 83, 46, 76, 77, 78, 79, 80, 81, or 82, respectively, and a CGRP-LCDR1, a CGRP-LCDR2, and a CGRP-LCDR3 in the CGRP-VL comprise the amino acid sequences of a CGRP-LCDR1, a CGRP-LCDR2, and a CGRP-LCDR3 in SEQ ID NO: 85, 47, or 84, respectively, or
(ii) a CGRP-HCDR1, a CGRP-HCDR2, and a CGRP-HCDR3 in the CGRP-VH comprise the amino acid sequences of a CGRP-HCDR1, a CGRP-HCDR2, and a CGRP-HCDR3 in SEQ ID NO: 72, 44, 68, 69, 70, or 71, respectively, and a CGRP-LCDR1, a CGRP-LCDR2, and a CGRP-LCDR3 in the CGRP-VL comprise the amino acid sequences of a CGRP-LCDR1, a CGRP-LCDR2, and a CGRP-LCDR3 in SEQ ID NO: 75, 45, 73, or 74, respectively, or
(iii) a CGRP-HCDR1, a CGRP-HCDR2, and a CGRP-HCDR3 in the CGRP-VH comprise the amino acid sequences of a CGRP-HCDR1, a CGRP-HCDR2, and a CGRP-HCDR3 in SEQ ID NO: 40, 54, 55, or 56, respectively, and a CGRP-LCDR1, a CGRP-LCDR2, and a CGRP-LCDR3 in the CGRP-VL comprise the amino acid sequences of a CGRP-LCDR1, a CGRP-LCDR2, and a CGRP-LCDR3 in SEQ ID NO: 41, 57, or 58, respectively, or
(iv) a CGRP-HCDR1, a CGRP-HCDR2, and a CGRP-HCDR3 in the CGRP-VH comprise the amino acid sequences of a CGRP-HCDR1, a CGRP-HCDR2, and a CGRP-HCDR3 in SEQ ID NO: 42, 59, 60, 61, 62, 63, 64, 65, or 66, respectively, and a CGRP-LCDR1, a CGRP-LCDR2, and a CGRP-LCDR3 in the CGRP-VL comprise the amino acid sequences of a CGRP-LCDR1, a CGRP-LCDR2, and a CGRP-LCDR3 in SEQ ID NO: 43 or 67, respectively.

In some embodiments, provided is the antigen-binding molecule described above, wherein the CGRP-HCDR1, CGRP-HCDR2, CGRP-HCDR3, CGRP-LCDR1, CGRP-LCDR2, and CGRP-LCDR3 are defined according to the Kabat, IMGT, Chothia, AbM, or Contact numbering scheme.

In some embodiments, provided is the antigen-binding molecule according to any one of the foregoing, wherein:
(i) the CGRP-VH comprises: a CGRP-HCDR1 comprising the amino acid sequence of SEQ ID NO: 22, a CGRP-HCDR2 comprising the amino acid sequence of SEQ ID NO: 106, 23, 103, 104, or 105, and a CGRP-HCDR3 comprising the amino acid sequence of SEQ ID NO: 24, and the CGRP-VL comprises: a CGRP-LCDR1 comprising the amino acid sequence of SEQ ID NO: 25, a CGRP-LCDR2 comprising the amino acid sequence of SEQ ID NO: 26, and a CGRP-LCDR3 comprising the amino acid sequence of SEQ ID NO: 27, or
(ii) the CGRP-VH comprises: a CGRP-HCDR1 comprising the amino acid sequence of SEQ ID NO: 16, a CGRP-HCDR2 comprising the amino acid sequence of SEQ ID NO: 101, 17, 100, 102, or 192, and a CGRP-HCDR3 comprising the amino acid sequence of SEQ ID NO: 18, and the CGRP-VL comprises: a CGRP-LCDR1 comprising the amino acid sequence of SEQ ID NO: 19, a CGRP-LCDR2 comprising the amino acid sequence of SEQ ID NO: 20, and a CGRP-LCDR3 comprising the amino acid sequence of SEQ ID NO: 21, or
(iii) the CGRP-VH comprises: a CGRP-HCDR1 comprising the amino acid sequence of SEQ ID NO: 4, a CGRP-HCDR2 comprising the amino acid sequence of SEQ ID NO: 5, 93, 94, or 95, and a CGRP-HCDR3 comprising the amino acid sequence of SEQ ID NO: 6, and the CGRP-VL comprises: a CGRP-LCDR1 comprising the amino acid sequence of SEQ ID NO: 7, a CGRP-LCDR2 comprising the amino acid sequence of SEQ ID NO: 8, and a CGRP-LCDR3 comprising the amino acid sequence of SEQ ID NO: 9, or
(iv) the CGRP-VH comprises: a CGRP-HCDR1 comprising the amino acid sequence of SEQ ID NO: 10, a CGRP-HCDR2 comprising the amino acid sequence of SEQ ID NO: 11, 96, 97, 98, or 99, and a CGRP-HCDR3 comprising the amino acid sequence of SEQ ID NO: 12, and the CGRP-VL comprises: a CGRP-LCDR1 comprising the amino acid sequence of SEQ ID NO: 13, a CGRP-LCDR2 comprising the amino acid sequence of SEQ ID NO: 14, and a CGRP-LCDR3 comprising the amino acid sequence of SEQ ID NO: 15.

In some embodiments, provided is the antigen-binding molecule according to any one of the foregoing, wherein:
the CGRP-VH comprises: a CGRP-HCDR1 comprising the amino acid sequence of SEQ ID NO: 22, a CGRP-HCDR2 comprising the amino acid sequence of SEQ ID NO: 106, and a CGRP-HCDR3 comprising the amino acid sequence of SEQ ID NO: 24, and the CGRP-VL comprises: a CGRP-LCDR1 comprising the amino acid sequence of SEQ ID NO: 25, a CGRP-LCDR2 comprising the amino acid sequence of SEQ ID NO: 26, and a CGRP-LCDR3 comprising the amino acid sequence of SEQ ID NO: 27, or
the CGRP-VH comprises: a CGRP-HCDR1 comprising the amino acid sequence of SEQ ID NO: 16, a CGRP-HCDR2 comprising the amino acid sequence of SEQ ID NO: 101, and a CGRP-HCDR3 comprising the amino acid sequence of SEQ ID NO: 18, and the CGRP-VL comprises: a CGRP-LCDR1 comprising the amino acid sequence of SEQ ID NO: 19, a CGRP-LCDR2 comprising the amino acid sequence of SEQ ID NO: 20, and a CGRP-LCDR3 comprising the amino acid sequence of SEQ ID NO: 21.

In some embodiments, provided is the antigen-binding molecule described above, wherein the CGRP-HCDR1, CGRP-HCDR2, CGRP-HCDR3, CGRP-LCDR1, CGRP-LCDR2, and CGRP-LCDR3 are defined according to the Kabat numbering scheme.

In some embodiments, provided is the antigen-binding molecule according to any one of the foregoing, wherein:
(i) the CGRP-VH comprises the amino acid sequence of SEQ ID NO: 83, 46, 76, 77, 78, 79, 80, 81, or 82, and the CGRP-VL comprises the amino acid sequence of SEQ ID NO: 85, 47, or 84, or
(ii) the CGRP-VH comprises the amino acid sequence of SEQ ID NO: 72, 44, 68, 69, 70, or 71, and the CGRP-VL comprises the amino acid sequence of SEQ ID NO: 75, 45, 73, or 74, or
(iii) the CGRP-VH comprises the amino acid sequence of SEQ ID NO: 40, 54, 55, or 56, and the CGRP-VL comprises the amino acid sequence of SEQ ID NO: 41, 57, or 58, or
(iv) the CGRP-VH comprises the amino acid sequence of SEQ ID NO: 42, 59, 60, 61, 62, 63, 64, 65, or 66, and the CGRP-VL comprises the amino acid sequence of SEQ ID NO: 43 or 67.

In some embodiments, provided is the antigen-binding molecule according to any one of the foregoing, wherein:
(i) the CGRP-VH comprises the amino acid sequence of SEQ ID NO: 83, 76, 77, 78, 79, 80, 81, or 82, and the CGRP-VL comprises the amino acid sequence of SEQ ID NO: 85 or 84; or the CGRP-VH comprises the amino acid sequence of SEQ ID NO: 46, and the CGRP-VL comprises the amino acid sequence of SEQ ID NO: 47; or
(ii) the CGRP-VH comprises the amino acid sequence of SEQ ID NO: 72, 68, 69, 70, or 71, and the CGRP-VL comprises the amino acid sequence of SEQ ID NO: 75, 73, or 74; or the CGRP-VH comprises the amino acid sequence of SEQ ID NO: 44, and the CGRP-VL comprises the amino acid sequence of SEQ ID NO: 45; or
(iii) the CGRP-VH comprises the amino acid sequence of SEQ ID NO: 54, 55, or 56, and the CGRP-VL comprises the amino acid sequence of SEQ ID NO: 57 or 58; or the CGRP-VH comprises the amino acid sequence of SEQ ID NO: 40, and the CGRP-VL comprises the amino acid sequence of SEQ ID NO: 41; or
(iv) the CGRP-VH comprises the amino acid sequence of SEQ ID NO: 59, 60, 61, 62, 63, 64, 65, or 66, and the CGRP-VL comprises the amino acid sequence of SEQ ID NO: 67; or the CGRP-VH comprises the amino acid sequence of SEQ ID NO: 42, and the CGRP-VL comprises the amino acid sequence of SEQ ID NO: 43.

In some embodiments, provided is the antigen-binding molecule according to any one of the foregoing, wherein:
(i) the CGRP-VH comprises the amino acid sequence of SEQ ID NO: 83, and the CGRP-VL comprises the amino acid sequence of SEQ ID NO: 85, or
the CGRP-VH comprises the amino acid sequence of SEQ ID NO: 46, and the CGRP-VL comprises the amino acid sequence of SEQ ID NO: 47, or
the CGRP-VH comprises the amino acid sequence of SEQ ID NO: 76, and the CGRP-VL comprises the amino acid sequence of SEQ ID NO: 84, or
the CGRP-VH comprises the amino acid sequence of SEQ ID NO: 77, and the CGRP-VL comprises the amino acid sequence of SEQ ID NO: 84, or
the CGRP-VH comprises the amino acid sequence of SEQ ID NO: 78, and the CGRP-VL comprises the amino acid sequence of SEQ ID NO: 84, or
the CGRP-VH comprises the amino acid sequence of SEQ ID NO: 79, and the CGRP-VL comprises the amino acid sequence of SEQ ID NO: 84, or
the CGRP-VH comprises the amino acid sequence of SEQ ID NO: 80, and the CGRP-VL comprises the amino acid sequence of SEQ ID NO: 84, or
the CGRP-VH comprises the amino acid sequence of SEQ ID NO: 81, and the CGRP-VL comprises the amino acid sequence of SEQ ID NO: 84, or
the CGRP-VH comprises the amino acid sequence of SEQ ID NO: 82, and the CGRP-VL comprises the amino acid sequence of SEQ ID NO: 84; or
(ii) the CGRP-VH comprises the amino acid sequence of SEQ ID NO: 72, and the CGRP-VL comprises the amino acid sequence of SEQ ID NO: 75, or
the CGRP-VH comprises the amino acid sequence of SEQ ID NO: 44, and the CGRP-VL comprises the amino acid sequence of SEQ ID NO: 45, or
the CGRP-VH comprises the amino acid sequence of SEQ ID NO: 68, and the CGRP-VL comprises the amino acid sequence of SEQ ID NO: 73, or
the CGRP-VH comprises the amino acid sequence of SEQ ID NO: 68, and the CGRP-VL comprises the amino acid sequence of SEQ ID NO: 74, or
the CGRP-VH comprises the amino acid sequence of SEQ ID NO: 69, and the CGRP-VL comprises the amino acid sequence of SEQ ID NO: 73, or
the CGRP-VH comprises the amino acid sequence of SEQ ID NO: 69, and the CGRP-VL comprises the amino acid sequence of SEQ ID NO: 74, or
the CGRP-VH comprises the amino acid sequence of SEQ ID NO: 70, and the CGRP-VL comprises the amino acid sequence of SEQ ID NO: 73, or
the CGRP-VH comprises the amino acid sequence of SEQ ID NO: 71, and the CGRP-VL comprises the amino acid sequence of SEQ ID NO: 73, or
the CGRP-VH comprises the amino acid sequence of SEQ ID NO: 71, and the CGRP-VL comprises the amino acid sequence of SEQ ID NO: 74; or
(iii) the CGRP-VH comprises the amino acid sequence of SEQ ID NO: 40, and the CGRP-VL comprises the amino acid sequence of SEQ ID NO: 41, or
the CGRP-VH comprises the amino acid sequence of SEQ ID NO: 54, and the CGRP-VL comprises the amino acid sequence of SEQ ID NO: 57, or
the CGRP-VH comprises the amino acid sequence of SEQ ID NO: 55, and the CGRP-VL comprises the amino acid sequence of SEQ ID NO: 57, or
the CGRP-VH comprises the amino acid sequence of SEQ ID NO: 56, and the CGRP-VL comprises the amino acid sequence of SEQ ID NO: 57, or
the CGRP-VH comprises the amino acid sequence of SEQ ID NO: 54, and the CGRP-VL comprises the amino acid sequence of SEQ ID NO: 58, or
the CGRP-VH comprises the amino acid sequence of SEQ ID NO: 56, and the CGRP-VL comprises the amino acid sequence of SEQ ID NO: 58; or
(iv) the CGRP-VH comprises the amino acid sequence of SEQ ID NO: 42, and the CGRP-VL comprises the amino acid sequence of SEQ ID NO: 43, or
the CGRP-VH comprises the amino acid sequence of SEQ ID NO: 59, and the CGRP-VL comprises the amino acid sequence of SEQ ID NO: 67, or
the CGRP-VH comprises the amino acid sequence of SEQ ID NO: 60, and the CGRP-VL comprises the amino acid sequence of SEQ ID NO: 67, or
the CGRP-VH comprises the amino acid sequence of SEQ ID NO: 61, and the CGRP-VL comprises the amino acid sequence of SEQ ID NO: 67, or
the CGRP-VH comprises the amino acid sequence of SEQ ID NO: 62, and the CGRP-VL comprises the amino acid sequence of SEQ ID NO: 67, or
the CGRP-VH comprises the amino acid sequence of SEQ ID NO: 63, and the CGRP-VL comprises the amino acid sequence of SEQ ID NO: 67, or
the CGRP-VH comprises the amino acid sequence of SEQ ID NO: 64, and the CGRP-VL comprises the amino acid sequence of SEQ ID NO: 67, or
the CGRP-VH comprises the amino acid sequence of SEQ ID NO: 65, and the CGRP-VL comprises the amino acid sequence of SEQ ID NO: 67, or
the CGRP-VH comprises the amino acid sequence of SEQ ID NO: 66, and the CGRP-VL comprises the amino acid sequence of SEQ ID NO: 67.

In some embodiments, provided is the antigen-binding molecule according to any one of the foregoing, wherein:
(i) the CGRP-VH comprises the amino acid sequence of SEQ ID NO: 83, and the CGRP-VL comprises the amino acid sequence of SEQ ID NO: 85, or
the CGRP-VH comprises the amino acid sequence of SEQ ID NO: 81, and the CGRP-VL comprises the amino acid sequence of SEQ ID NO: 84; or
(ii) the CGRP-VH comprises the amino acid sequence of SEQ ID NO: 72, and the CGRP-VL comprises the amino acid sequence of SEQ ID NO: 75.

In some embodiments, provided is the antigen-binding molecule according to any one of the foregoing, wherein:
(v) a PACAP-HCDR1, a PACAP-HCDR2, and a PACAP-HCDR3 in the PACAP-VH comprise the amino acid sequences of a PACAP-HCDR1, a PACAP-HCDR2, and a PACAP-HCDR3 in SEQ ID NO: 87, 48, or 86, respectively, and a PACAP-LCDR1, a PACAP-LCDR2, and a PACAP-LCDR3 in the PACAP-VL comprise the amino acid sequences of a PACAP-LCDR1, a PACAP-LCDR2, and a PACAP-LCDR3 in SEQ ID NO: 90, 49, 88, or 89, respectively, or
(vi) a PACAP-HCDR1, a PACAP-HCDR2, and a PACAP-HCDR3 in the PACAP-VH comprise the amino acid sequences of a PACAP-HCDR1, a PACAP-HCDR2, and a PACAP-HCDR3 in SEQ ID NO: 50 or 91, respectively, and a PACAP-LCDR1, a PACAP-LCDR2, and a PACAP-LCDR3 in the PACAP-VL comprise the amino acid sequences of a PACAP-LCDR1, a PACAP-LCDR2, and a PACAP-LCDR3 in SEQ ID NO: 51 or 92, respectively.

In some embodiments, provided is the antigen-binding molecule described above, wherein the PACAP-HCDR1, PACAP-HCDR2, PACAP-HCDR3, PACAP-LCDR1, PACAP-LCDR2, and PACAP-LCDR3 are defined according to the Kabat, IMGT, Chothia, AbM, or Contact numbering scheme.

In some embodiments, provided is the antigen-binding molecule according to any one of the foregoing, wherein:
(v) the PACAP-VH comprises: a PACAP-HCDR1 comprising the amino acid sequence of SEQ ID NO: 28, a PACAP-HCDR2 comprising the amino acid sequence of SEQ ID NO: 29, and a PACAP-HCDR3 comprising the amino acid sequence of SEQ ID NO: 107 or 30, and the PACAP-VL comprises: a PACAP-LCDR1 comprising the amino acid sequence of SEQ ID NO: 31, a PACAP-LCDR2 comprising the amino acid sequence of SEQ ID NO: 108 or 32, and a PACAP-LCDR3 comprising the amino acid sequence of SEQ ID NO: 33; or
(vi) the PACAP-VH comprises: a PACAP-HCDR1 comprising the amino acid sequence of SEQ ID NO: 34, a PACAP-HCDR2 comprising the amino acid sequence of SEQ ID NO: 35 or 109, and a PACAP-HCDR3 comprising the amino acid sequence of SEQ ID NO: 36, and the PACAP-VL comprises: a PACAP-LCDR1 comprising the amino acid sequence of SEQ ID NO: 37, a PACAP-LCDR2 comprising the amino acid sequence of SEQ ID NO: 38, and a PACAP-LCDR3 comprising the amino acid sequence of SEQ ID NO: 39.

In some embodiments, provided is the antigen-binding molecule according to any one of the foregoing, wherein the PACAP-VH comprises: a PACAP-HCDR1 comprising the amino acid sequence of SEQ ID NO: 28, a PACAP-HCDR2 comprising the amino acid sequence of SEQ ID NO: 29, and a PACAP-HCDR3 comprising the amino acid sequence of SEQ ID NO: 107, and the PACAP-VL comprises: a PACAP-LCDR1 comprising the amino acid sequence of SEQ ID NO: 31, a PACAP-LCDR2 comprising the amino acid sequence of SEQ ID NO: 108, and a PACAP-LCDR3 comprising the amino acid sequence of SEQ ID NO: 33.

In some embodiments, provided is the antigen-binding molecule described above, wherein the PACAP-HCDR1, PACAP-HCDR2, PACAP-HCDR3, PACAP-LCDR1, PACAP-LCDR2, and PACAP-LCDR3 are defined according to the Kabat numbering scheme.

In some embodiments, provided is the antigen-binding molecule according to any one of the foregoing, wherein:
(v) the PACAP-VH comprises the amino acid sequence of SEQ ID NO: 87, 48, or 86, and the PACAP-VL comprises the amino acid sequence of SEQ ID NO: 90, 49, 88, or 89, or
(vi) the PACAP-VH comprises the amino acid sequence of SEQ ID NO: 50 or 91, and the PACAP-VL comprises the amino acid sequence of SEQ ID NO: 51 or 92.

In some embodiments, provided is the antigen-binding molecule according to any one of the foregoing, wherein:
(v) the PACAP-VH comprises the amino acid sequence of SEQ ID NO: 87 or 86, and the PACAP-VL comprises the amino acid sequence of SEQ ID NO: 90, 88, or 89; or the PACAP-VH comprises the amino acid sequence of SEQ ID NO: 48, and the PACAP-VL comprises the amino acid sequence of SEQ ID NO: 49; or
(vi) the PACAP-VH comprises the amino acid sequence of SEQ ID NO: 91, and the PACAP-VL comprises the amino acid sequence of SEQ ID NO: 92; or the PACAP-VH comprises the amino acid sequence of SEQ ID NO: 50, and the PACAP-VL comprises the amino acid sequence of SEQ ID NO: 51.

In some embodiments, provided is the antigen-binding molecule according to any one of the foregoing, wherein:
(v) the PACAP-VH comprises the amino acid sequence of SEQ ID NO: 87, and the PACAP-VL comprises the amino acid sequence of SEQ ID NO: 90, or
the PACAP-VH comprises the amino acid sequence of SEQ ID NO: 48, and the PACAP-VL comprises the amino acid sequence of SEQ ID NO: 49, or
the PACAP-VH comprises the amino acid sequence of SEQ ID NO: 86, and the PACAP-VL comprises the amino acid sequence of SEQ ID NO: 88, or
the PACAP-VH comprises the amino acid sequence of SEQ ID NO: 86, and the PACAP-VL comprises the amino acid sequence of SEQ ID NO: 89; or
(vi) the PACAP-VH comprises the amino acid sequence of SEQ ID NO: 50, and the PACAP-VL comprises the amino acid sequence of SEQ ID NO: 51, or
the PACAP-VH comprises the amino acid sequence of SEQ ID NO: 91, and the PACAP-VL comprises the amino acid sequence of SEQ ID NO: 92.

In some embodiments, provided is the antigen-binding molecule according to any one of the foregoing, wherein:
(v) the PACAP-VH comprises the amino acid sequence of SEQ ID NO: 87, and the PACAP-VL comprises the amino acid sequence of SEQ ID NO: 90.

In some embodiments, provided is the antigen-binding molecule according to any one of the foregoing, wherein the antigen-binding molecule comprises an Fc region (including an IgG Fc region or an IgG1 Fc region). In some embodiments, the Fc region comprises one or more amino acid substitutions capable of increasing the serum half-life of the antigen-binding molecule. In some embodiments, the Fc region is a human IgG1 Fc region, with the amino acid residue at position 252 being Y, the amino acid residue at position 254 being T, and the amino acid residue at position 256 being E, as numbered according to the EU index.

In some embodiments, provided is the antigen-binding molecule according to any one of the foregoing, wherein the antigen-binding molecule comprises two antigen-binding moieties that specifically bind to CGRP, two antigen-binding moieties that specifically bind to PACAP, and an Fc region.

In some embodiments, provided is the antigen-binding molecule according to any one of the foregoing, wherein the antigen-binding molecule comprises two first chains having a structure represented by formula (a) and two second chains having a structure represented by formula (b):

[PACAP-VH]-[CH1]-[CGRP-VH]-[linker 1]-[CGRP-VL]-[linker 2]-[one subunit of the Fc region], and formula (a)

[PACAP-VL]-[CL], formula (b)

wherein the structures represented by formulas (a) and (b) are arranged from the N-terminus to the C-terminus, and the linker 1 and the linker 2 are identical or different peptide linkers.

In some embodiments, provided is the antigen-binding molecule according to any one of the foregoing, wherein the antigen-binding molecule comprises: two first chains comprising the amino acid sequence of SEQ ID NO: 113 and two second chains comprising the amino acid sequence of SEQ ID NO: 114; or
the antigen-binding molecule comprises: two first chains comprising the amino acid sequence of SEQ ID NO: 194 and two second chains comprising the amino acid sequence of SEQ ID NO: 114; or
the antigen-binding molecule comprises: two first chains comprising the amino acid sequence of SEQ ID NO: 111 and two second chains comprising the amino acid sequence of SEQ ID NO: 112.

In some embodiments, provided is the antigen-binding molecule according to any one of the foregoing, wherein the antigen-binding molecule comprises one antigen-binding moiety that specifically binds to CGRP, one antigen-binding moiety that specifically binds to PACAP, and an Fc region, wherein the Fc region comprises a first subunit Fc1 and a second subunit Fc2 that are capable of associating with each other, and the Fc1 and Fc2 each independently have one or more amino acid substitutions that reduce homodimerization of the Fc region.

In some embodiments, the Fc1 has a knob structure according to the knob-into-hole technique, and the Fc2 has a hole structure according to the knob-into-hole technique.

In some embodiments, the amino acid residue at position 366 of the Fc1 is W; and the amino acid residue at position 366 of the Fc2 is S, the amino acid residue at position 368 is A, and the amino acid residue at position 407 is V, as numbered according to the EU index.

In some embodiments, the amino acid residue at position 354 of the Fc1 is C, and the amino acid residue at position 349 of the Fc2 is C, as numbered according to the EU index.

In some embodiments, one of the antigen-binding moiety that specifically binds to CGRP and the antigen-binding moiety that specifically binds to PACAP comprises a Titin chain and an Obscurin chain that are capable of forming a dimer. In some embodiments, the Titin chain comprises an amino acid sequence selected from the group consisting of SEQ ID NO: 121 to SEQ ID NO: 139, and the Obscurin chain comprises an amino acid sequence selected from the group consisting of SEQ ID NO: 140 to SEQ ID NO: 180. In some embodiments, the Titin chain comprises the amino acid sequence of SEQ ID NO: 137, and the Obscurin chain comprises the amino acid sequence of SEQ ID NO: 175.

In some embodiments, provided is the antigen-binding molecule according to any one of the foregoing, wherein the antigen-binding molecule comprises one first chain having a structure represented by formula (c), one second chain having a structure represented by formula (b), one third chain having a structure represented by formula (d), and one fourth chain having a structure represented by formula (e):

[PACAP-VH]-[CH1]-[Fc1], formula (c)

[PACAP-VL]-[CL], formula (b)

[CGRP-VH]-[linker 3]-[Titin chain]-[Fc2], and formula (d)

[CGRP-VL]-[linker 4]-[Obscurin chain], formula (e)

wherein the structures represented by formulas (c), (b), (d), and (e) are arranged from the N-terminus to the C-terminus, and the linker 3 and the linker 4 are identical or different peptide linkers.

In some embodiments, provided is the antigen-binding molecule according to any one of the foregoing, wherein the antigen-binding molecule comprises: a first chain comprising the amino acid sequence of SEQ ID NO: 117, a second chain comprising the amino acid sequence of SEQ ID NO: 118, a third chain comprising the amino acid sequence of SEQ ID NO: 119, and a fourth chain comprising the amino acid sequence of SEQ ID NO: 120.

In another aspect, the present disclosure further provides an isolated antibody capable of specifically binding to CGRP, wherein the antibody comprises a heavy chain variable region CGRP-VH and a light chain variable region CGRP-VL, wherein:
(i) a CGRP-HCDR1, a CGRP-HCDR2, and a CGRP-HCDR3 in the CGRP-VH comprise the amino acid sequences of a CGRP-HCDR1, a CGRP-HCDR2, and a CGRP-HCDR3 in SEQ ID NO: 83, 46, 76, 77, 78, 79, 80, 81, or 82, respectively, and a CGRP-LCDR1, a CGRP-LCDR2, and a CGRP-LCDR3 in the CGRP-VL comprise the amino acid sequences of a CGRP-LCDR1, a CGRP-LCDR2, and a CGRP-LCDR3 in SEQ ID NO: 85, 47, or 84, respectively, or
(ii) a CGRP-HCDR1, a CGRP-HCDR2, and a CGRP-HCDR3 in the CGRP-VH comprise the amino acid sequences of a CGRP-HCDR1, a CGRP-HCDR2, and a CGRP-HCDR3 in SEQ ID NO: 72, 44, 68, 69, 70, or 71, respectively, and a CGRP-LCDR1, a CGRP-LCDR2, and a CGRP-LCDR3 in the CGRP-VL comprise the amino acid sequences of a CGRP-LCDR1, a CGRP-LCDR2, and a CGRP-LCDR3 in SEQ ID NO: 75, 45, 73, or 74, respectively, or
(iii) a CGRP-HCDR1, a CGRP-HCDR2, and a CGRP-HCDR3 in the CGRP-VH comprise the amino acid sequences of a CGRP-HCDR1, a CGRP-HCDR2, and a CGRP-HCDR3 in SEQ ID NO: 40, 54, 55, or 56, respectively, and a CGRP-LCDR1, a CGRP-LCDR2, and a CGRP-LCDR3 in the CGRP-VL comprise the amino acid sequences of a CGRP-LCDR1, a CGRP-LCDR2, and a CGRP-LCDR3 in SEQ ID NO: 41, 57, or 58, respectively, or
(iv) a CGRP-HCDR1, a CGRP-HCDR2, and a CGRP-HCDR3 in the CGRP-VH comprise the amino acid sequences of a CGRP-HCDR1, a CGRP-HCDR2, and a CGRP-HCDR3 in SEQ ID NO: 42, 59, 60, 61, 62, 63, 64, 65, or 66, respectively, and a CGRP-LCDR1, a CGRP-LCDR2, and a CGRP-LCDR3 in the CGRP-VL comprise the amino acid sequences of a CGRP-LCDR1, a CGRP-LCDR2, and a CGRP-LCDR3 in SEQ ID NO: 43 or 67, respectively.

In some embodiments, provided is the isolated antibody according to any one of the foregoing, wherein:
(i) the CGRP-VH comprises: a CGRP-HCDR1 comprising the amino acid sequence of SEQ ID NO: 22, a CGRP-HCDR2 comprising the amino acid sequence of SEQ ID NO: 106, 23, 103, 104, or 105, and a CGRP-HCDR3 comprising the amino acid sequence of SEQ ID NO: 24, and the CGRP-VL comprises: a CGRP-LCDR1 comprising the amino acid sequence of SEQ ID NO: 25, a CGRP-LCDR2 comprising the amino acid sequence of SEQ ID NO: 26, and a CGRP-LCDR3 comprising the amino acid sequence of SEQ ID NO: 27, or
(ii) the CGRP-VH comprises: a CGRP-HCDR1 comprising the amino acid sequence of SEQ ID NO: 16, a CGRP-HCDR2 comprising the amino acid sequence of SEQ ID NO: 101, 17, 100, 102, or 192, and a CGRP-HCDR3 comprising the amino acid sequence of SEQ ID NO: 18, and the CGRP-VL comprises: a CGRP-LCDR1 comprising the amino acid sequence of SEQ ID NO: 19, a CGRP-LCDR2 comprising the amino acid sequence of SEQ ID NO: 20, and a CGRP-LCDR3 comprising the amino acid sequence of SEQ ID NO: 21, or
(iii) the CGRP-VH comprises: a CGRP-HCDR1 comprising the amino acid sequence of SEQ ID NO: 4, a CGRP-HCDR2 comprising the amino acid sequence of SEQ ID NO: 5, 93, 94, or 95, and a CGRP-HCDR3 comprising the amino acid sequence of SEQ ID NO: 6, and the CGRP-VL comprises: a CGRP-LCDR1 comprising the amino acid sequence of SEQ ID NO: 7, a CGRP-LCDR2 comprising the amino acid sequence of SEQ ID NO: 8, and a CGRP-LCDR3 comprising the amino acid sequence of SEQ ID NO: 9, or
(iv) the CGRP-VH comprises: a CGRP-HCDR1 comprising the amino acid sequence of SEQ ID NO: 10, a CGRP-HCDR2 comprising the amino acid sequence of SEQ ID NO: 11, 96, 97, 98, or 99, and a CGRP-HCDR3 comprising the amino acid sequence of SEQ ID NO: 12, and the CGRP-VL comprises: a CGRP-LCDR1 comprising the amino acid sequence of SEQ ID NO: 13, a CGRP-LCDR2 comprising the amino acid sequence of SEQ ID NO: 14, and a CGRP-LCDR3 comprising the amino acid sequence of SEQ ID NO: 15.

In some embodiments, provided is the isolated antibody according to any one of the foregoing, wherein:
the CGRP-VH comprises: a CGRP-HCDR1 comprising the amino acid sequence of SEQ ID NO: 22, a CGRP-HCDR2 comprising the amino acid sequence of SEQ ID NO: 106, and a CGRP-HCDR3 comprising the amino acid sequence of SEQ ID NO: 24, and the CGRP-VL comprises: a CGRP-LCDR1 comprising the amino acid sequence of SEQ ID NO: 25, a CGRP-LCDR2 comprising the amino acid sequence of SEQ ID NO: 26, and a CGRP-LCDR3 comprising the amino acid sequence of SEQ ID NO: 27, or
the CGRP-VH comprises: a CGRP-HCDR1 comprising the amino acid sequence of SEQ ID NO: 16, a CGRP-HCDR2 comprising the amino acid sequence of SEQ ID NO: 101, and a CGRP-HCDR3 comprising the amino acid sequence of SEQ ID NO: 18, and the CGRP-VL comprises: a CGRP-LCDR1 comprising the amino acid sequence of SEQ ID NO: 19, a CGRP-LCDR2 comprising the amino acid sequence of SEQ ID NO: 20, and a CGRP-LCDR3 comprising the amino acid sequence of SEQ ID NO: 21.

In some embodiments, provided is the isolated antibody according to any one of the foregoing, wherein:
(i) the CGRP-VH comprises the amino acid sequence of SEQ ID NO: 83, 46, 76, 77, 78, 79, 80, 81, or 82, and the CGRP-VL comprises the amino acid sequence of SEQ ID NO: 85, 47, or 84, or
(ii) the CGRP-VH comprises the amino acid sequence of SEQ ID NO: 72, 44, 68, 69, 70, or 71, and the CGRP-VL comprises the amino acid sequence of SEQ ID NO: 75, 45, 73, or 74, or
(iii) the CGRP-VH comprises the amino acid sequence of SEQ ID NO: 40, 54, 55, or 56, and the CGRP-VL comprises the amino acid sequence of SEQ ID NO: 41, 57, or 58, or
(iv) the CGRP-VH comprises the amino acid sequence of SEQ ID NO: 42, 59, 60, 61, 62, 63, 64, 65, or 66, and the CGRP-VL comprises the amino acid sequence of SEQ ID NO: 43 or 67.

In some embodiments, provided is the isolated antibody according to any one of the foregoing, wherein:
(i) the CGRP-VH comprises the amino acid sequence of SEQ ID NO: 83, and the CGRP-VL comprises the amino acid sequence of SEQ ID NO: 85, or
the CGRP-VH comprises the amino acid sequence of SEQ ID NO: 46, and the CGRP-VL comprises the amino acid sequence of SEQ ID NO: 47, or
the CGRP-VH comprises the amino acid sequence of SEQ ID NO: 76, and the CGRP-VL comprises the amino acid sequence of SEQ ID NO: 84, or
the CGRP-VH comprises the amino acid sequence of SEQ ID NO: 77, and the CGRP-VL comprises the amino acid sequence of SEQ ID NO: 84, or
the CGRP-VH comprises the amino acid sequence of SEQ ID NO: 78, and the CGRP-VL comprises the amino acid sequence of SEQ ID NO: 84, or
the CGRP-VH comprises the amino acid sequence of SEQ ID NO: 79, and the CGRP-VL comprises the amino acid sequence of SEQ ID NO: 84, or
the CGRP-VH comprises the amino acid sequence of SEQ ID NO: 80, and the CGRP-VL comprises the amino acid sequence of SEQ ID NO: 84, or
the CGRP-VH comprises the amino acid sequence of SEQ ID NO: 81, and the CGRP-VL comprises the amino acid sequence of SEQ ID NO: 84, or
the CGRP-VH comprises the amino acid sequence of SEQ ID NO: 82, and the CGRP-VL comprises the amino acid sequence of SEQ ID NO: 84; or
(ii) the CGRP-VH comprises the amino acid sequence of SEQ ID NO: 72, and the CGRP-VL comprises the amino acid sequence of SEQ ID NO: 75, or
the CGRP-VH comprises the amino acid sequence of SEQ ID NO: 44, and the CGRP-VL comprises the amino acid sequence of SEQ ID NO: 45, or
the CGRP-VH comprises the amino acid sequence of SEQ ID NO: 68, and the CGRP-VL comprises the amino acid sequence of SEQ ID NO: 73, or
the CGRP-VH comprises the amino acid sequence of SEQ ID NO: 68, and the CGRP-VL comprises the amino acid sequence of SEQ ID NO: 74, or
the CGRP-VH comprises the amino acid sequence of SEQ ID NO: 69, and the CGRP-VL comprises the amino acid sequence of SEQ ID NO: 73, or
the CGRP-VH comprises the amino acid sequence of SEQ ID NO: 69, and the CGRP-VL comprises the amino acid sequence of SEQ ID NO: 74, or
the CGRP-VH comprises the amino acid sequence of SEQ ID NO: 70, and the CGRP-VL comprises the amino acid sequence of SEQ ID NO: 73, or
the CGRP-VH comprises the amino acid sequence of SEQ ID NO: 71, and the CGRP-VL comprises the amino acid sequence of SEQ ID NO: 73, or
the CGRP-VH comprises the amino acid sequence of SEQ ID NO: 71, and the CGRP-VL comprises the amino acid sequence of SEQ ID NO: 74; or
(iii) the CGRP-VH comprises the amino acid sequence of SEQ ID NO: 40, and the CGRP-VL comprises the amino acid sequence of SEQ ID NO: 41, or
the CGRP-VH comprises the amino acid sequence of SEQ ID NO: 54, and the CGRP-VL comprises the amino acid sequence of SEQ ID NO: 57, or
the CGRP-VH comprises the amino acid sequence of SEQ ID NO: 55, and the CGRP-VL comprises the amino acid sequence of SEQ ID NO: 57, or
the CGRP-VH comprises the amino acid sequence of SEQ ID NO: 56, and the CGRP-VL comprises the amino acid sequence of SEQ ID NO: 57, or
the CGRP-VH comprises the amino acid sequence of SEQ ID NO: 54, and the CGRP-VL comprises the amino acid sequence of SEQ ID NO: 58, or
the CGRP-VH comprises the amino acid sequence of SEQ ID NO: 56, and the CGRP-VL comprises the amino acid sequence of SEQ ID NO: 58; or
(iv) the CGRP-VH comprises the amino acid sequence of SEQ ID NO: 42, and the CGRP-VL comprises the amino acid sequence of SEQ ID NO: 43, or
the CGRP-VH comprises the amino acid sequence of SEQ ID NO: 59, and the CGRP-VL comprises the amino acid sequence of SEQ ID NO: 67, or
the CGRP-VH comprises the amino acid sequence of SEQ ID NO: 60, and the CGRP-VL comprises the amino acid sequence of SEQ ID NO: 67, or
the CGRP-VH comprises the amino acid sequence of SEQ ID NO: 61, and the CGRP-VL comprises the amino acid sequence of SEQ ID NO: 67, or
the CGRP-VH comprises the amino acid sequence of SEQ ID NO: 62, and the CGRP-VL comprises the amino acid sequence of SEQ ID NO: 67, or
the CGRP-VH comprises the amino acid sequence of SEQ ID NO: 63, and the CGRP-VL comprises the amino acid sequence of SEQ ID NO: 67, or
the CGRP-VH comprises the amino acid sequence of SEQ ID NO: 64, and the CGRP-VL comprises the amino acid sequence of SEQ ID NO: 67, or
the CGRP-VH comprises the amino acid sequence of SEQ ID NO: 65, and the CGRP-VL comprises the amino acid sequence of SEQ ID NO: 67, or
the CGRP-VH comprises the amino acid sequence of SEQ ID NO: 66, and the CGRP-VL comprises the amino acid sequence of SEQ ID NO: 67.

In some embodiments, provided is the isolated antibody according to any one of the foregoing, wherein:
(i) the CGRP-VH comprises the amino acid sequence of SEQ ID NO: 83, and the CGRP-VL comprises the amino acid sequence of SEQ ID NO: 85, or
the CGRP-VH comprises the amino acid sequence of SEQ ID NO: 81, and the CGRP-VL comprises the amino acid sequence of SEQ ID NO: 84; or
(ii) the CGRP-VH comprises the amino acid sequence of SEQ ID NO: 72, and the CGRP-VL comprises the amino acid sequence of SEQ ID NO: 75.

In another aspect, the present disclosure further provides an isolated antibody capable of specifically binding to PACAP, wherein the antibody comprises a heavy chain variable region PACAP-VH and a light chain variable region PACAP-VL, wherein:
(v) a PACAP-HCDR1, a PACAP-HCDR2, and a PACAP-HCDR3 in the PACAP-VH comprise the amino acid sequences of a PACAP-HCDR1, a PACAP-HCDR2, and a PACAP-HCDR3 in SEQ ID NO: 87, 48, or 86, respectively, and a PACAP-LCDR1, a PACAP-LCDR2, and a PACAP-LCDR3 in the PACAP-VL comprise the amino acid sequences of a PACAP-LCDR1, a PACAP-LCDR2, and a PACAP-LCDR3 in SEQ ID NO: 90, 49, 88, or 89, respectively, or
(vi) a PACAP-HCDR1, a PACAP-HCDR2, and a PACAP-HCDR3 in the PACAP-VH comprise the amino acid sequences of a PACAP-HCDR1, a PACAP-HCDR2, and a PACAP-HCDR3 in SEQ ID NO: 50 or 91, respectively, and a PACAP-LCDR1, a PACAP-LCDR2, and a PACAP-LCDR3 in the PACAP-VL comprise the amino acid sequences of a PACAP-LCDR1, a PACAP-LCDR2, and a PACAP-LCDR3 in SEQ ID NO: 51 or 92, respectively.

In some embodiments, provided is the isolated antibody according to any one of the foregoing, wherein:
(v) the PACAP-VH comprises: a PACAP-HCDR1 comprising the amino acid sequence of SEQ ID NO: 28, a PACAP-HCDR2 comprising the amino acid sequence of SEQ ID NO: 29, and a PACAP-HCDR3 comprising the amino acid sequence of SEQ ID NO: 107 or 30, and the PACAP-VL comprises: a PACAP-LCDR1 comprising the amino acid sequence of SEQ ID NO: 31, a PACAP-LCDR2 comprising the amino acid sequence of SEQ ID NO: 108 or 32, and a PACAP-LCDR3 comprising the amino acid sequence of SEQ ID NO: 33; or
(vi) the PACAP-VH comprises: a PACAP-HCDR1 comprising the amino acid sequence of SEQ ID NO: 34, a PACAP-HCDR2 comprising the amino acid sequence of SEQ ID NO: 35 or 109, and a PACAP-HCDR3 comprising the amino acid sequence of SEQ ID NO: 36, and the PACAP-VL comprises: a PACAP-LCDR1 comprising the amino acid sequence of SEQ ID NO: 37, a PACAP-LCDR2 comprising the amino acid sequence of SEQ ID NO: 38, and a PACAP-LCDR3 comprising the amino acid sequence of SEQ ID NO: 39.

In some embodiments, provided is the isolated antibody according to any one of the foregoing, wherein:
the PACAP-VH comprises: a PACAP-HCDR1 comprising the amino acid sequence of SEQ ID NO: 28, a PACAP-HCDR2 comprising the amino acid sequence of SEQ ID NO: 29, and a PACAP-HCDR3 comprising the amino acid sequence of SEQ ID NO: 107, and the PACAP-VL comprises: a PACAP-LCDR1 comprising the amino acid sequence of SEQ ID NO: 31, a PACAP-LCDR2 comprising the amino acid sequence of SEQ ID NO: 108, and a PACAP-LCDR3 comprising the amino acid sequence of SEQ ID NO: 33.

In some embodiments, provided is the isolated antibody according to any one of the foregoing, wherein:
(v) the PACAP-VH comprises the amino acid sequence of SEQ ID NO: 87, 48, or 86, and the PACAP-VL comprises the amino acid sequence of SEQ ID NO: 90, 49, 88, or 89, or (vi) the PACAP-VH comprises the amino acid sequence of SEQ ID NO: 50 or 91, and the PACAP-VL comprises the amino acid sequence of SEQ ID NO: 51 or 92.

In some embodiments, provided is the isolated antibody according to any one of the foregoing, wherein:
(v) the PACAP-VH comprises the amino acid sequence of SEQ ID NO: 87, and the PACAP-VL comprises the amino acid sequence of SEQ ID NO: 90, or
the PACAP-VH comprises the amino acid sequence of SEQ ID NO: 48, and the PACAP-VL comprises the amino acid sequence of SEQ ID NO: 49, or
the PACAP-VH comprises the amino acid sequence of SEQ ID NO: 86, and the PACAP-VL comprises the amino acid sequence of SEQ ID NO: 88, or
the PACAP-VH comprises the amino acid sequence of SEQ ID NO: 86, and the PACAP-VL comprises the amino acid sequence of SEQ ID NO: 89; or
(vi) the PACAP-VH comprises the amino acid sequence of SEQ ID NO: 50, and the PACAP-VL comprises the amino acid sequence of SEQ ID NO: 51, or
the PACAP-VH comprises the amino acid sequence of SEQ ID NO: 91, and the PACAP-VL comprises the amino acid sequence of SEQ ID NO: 92.

In some embodiments, provided is the isolated antibody according to any one of the foregoing, wherein the PACAP-VH comprises the amino acid sequence of SEQ ID NO: 87, and the PACAP-VL comprises the amino acid sequence of SEQ ID NO: 90.

In some embodiments, provided is the isolated antibody according to any one of the foregoing, wherein the isolated antibody is a bispecific antibody. In some embodiments, the bispecific antibody specifically binds to CGRP and PACAP.

In another aspect, the present disclosure provides a pharmaceutical composition comprising: a therapeutically effective amount of the antigen-binding molecule according to any one of the foregoing or the isolated antibody according to any one of the foregoing, and one or more pharmaceutically acceptable vehicles, diluents, buffers, or excipients. In some embodiments, the pharmaceutical composition further comprises at least one second therapeutic agent.

In another aspect, the present disclosure further provides an isolated nucleic acid encoding the antigen-binding molecule according to any one of the foregoing or the isolated antibody according to any one of the foregoing.

In another aspect, the present disclosure further provides a host cell comprising the aforementioned isolated nucleic acid.

In another aspect, the present disclosure further provides a method for treating a disease, the method comprising administering to a subject a therapeutically effective amount of the antigen-binding molecule according to any one of the foregoing or the isolated antibody according to any one of the foregoing or the composition thereof.

In another aspect, the present disclosure further provides use of the antigen-binding molecule according to any one of the foregoing or the isolated antibody according to any one of the foregoing or the composition thereof in the manufacture of a medicament for treating or preventing a disease.

In another aspect, the present disclosure further provides the antigen-binding molecule according to any one of the foregoing or the antibody according to any one of the foregoing or the composition thereof for use as a medicament. In some embodiments, the medicament is used for treating a disease.

In some embodiments, the disease according to any one of the foregoing is pain. In some embodiments, the disease according to any one of the foregoing is PACAP and/or CGRP-related pain. In some embodiments, the disease is headache. In some embodiments, the the disease is migraine or cluster headache.

The antigen-binding molecule provided in the present disclosure has the characteristics of good therapeutic activity, a good safety profile, a good pharmacokinetic profile, and good druggability (e.g., stability).

### BRIEF DESCRIPTION OF THE DRAWINGS

FIG. 1A: a structural schematic diagram of Format2+2.

FIG. 1B: a structural schematic diagram of Format1+1.

### DETAILED DESCRIPTION

### Terminology

The terminology used herein is for the purpose of describing embodiments only and is not intended to be limiting. Unless defined otherwise, all technical and scientific terms used herein have the same meaning as commonly understood by one of ordinary skill in the art to which the present disclosure pertains.

Unless the context clearly requires otherwise, throughout the description and the claims, the words “comprise”, “have”, “include”, and the like are to be understood in an inclusive sense as opposed to an exclusive or exhaustive sense; that is to say, in the sense of “including, but not limited to”. Unless otherwise stated, “comprise” includes “consist of”. For example, for a CGRP-HCDR1 comprising the amino acid sequence of SEQ ID NO: 22, it specifically encompasses a CGRP-HCDR1 whose amino acid sequence is set forth in SEQ ID NO: 22.

The three-letter and single-letter codes used in the present disclosure for amino acids are as described in J Biol. Chem, 243, p 3558 (1968).

The term “and/or”, e.g., “X and/or Y” should be understood to mean “X and Y” or “X or Y” and should be used to provide explicit support for both meanings or either meaning.

The term “amino acid” refers to naturally occurring and synthetic amino acids, as well as amino acid analogs and amino acid mimics that function similarly to the naturally occurring amino acids. Naturally occurring amino acids are those encoded by genetic codes and those amino acids later modified, e.g., hydroxyproline, γ-carboxyglutamic acid, and O-phosphoserine. Amino acid analogs refer to compounds that have a substantially identical chemical structure (i.e., an a carbon that binds to hydrogen, carboxyl, amino, and an R group) to naturally occurring amino acids, e.g., homoserine, norleucine, methionine sulfoxide, and methioninemethyl sulfonium. Such analogs have a modified R group (e.g., norleucine) or a modified peptide skeleton, but retain a substantially identical chemical structure to naturally occurring amino acids. Amino acid mimics refer to chemical compounds that have a structure different from the general chemical structure of amino acids, but function similarly to naturally occurring amino acids.

The term “amino acid mutation” includes amino acid substitutions, deletions, insertions, and modifications. Any combination of substitutions, deletions, insertions, and modifications can be made to obtain a final construct, as long as the final construct possesses the desired properties, such as reduced or binding to an Fc receptor. Amino acid sequence deletions and insertions may be deletions and insertions at the amino-terminus and/or the carboxyl-terminus of a polypeptide chain. Specific amino acid mutations may be amino acid substitutions. In one embodiment, the amino acid mutation is a non-conservative amino acid substitution, i.e., the replacement of one amino acid with another amino acid having different structural and/or chemical properties. Amino acid substitutions include replacement with non-naturally occurring amino acids or with derivatives of the 20 natural amino acids (e.g., 4-hydroxyproline, 3-methylhistidine, ornithine, homoserine, and 5-hydroxylysine). Amino acid mutations can be generated using genetic or chemical methods well known in the art. Genetic methods may include site-directed mutagenesis, PCR, gene synthesis, and the like. It is contemplated that methods for altering amino acid side chain groups other than genetic engineering, such as chemical modification, may also be used. Various names may be used herein to indicate the same amino acid mutation. Herein, the expression of “position+amino acid residue” may be used to denote an amino acid residue at a specific position. For example, 366W indicates that the amino acid residue at position 366 is W. T366W indicates that an amino acid residue at position 366 is mutated from the original T to W.

The term “antigen-binding molecule” is used in the broadest sense and encompasses a variety of molecules that specifically bind to an antigen (or an epitope thereof), including but not limited to antibodies, polypeptides with antigen-binding activity, and antibody fusion proteins in which the two are fused, as long as they exhibit desired antigen-binding activity. The antigen-binding molecule herein comprises a variable region (VH) and a variable region (VL) which together comprise an antigen-binding domain. Illustratively, the antigen-binding molecule herein is a bispecific antigen-binding molecule (e.g., a bispecific antibody).

The term “antibody” is used in the broadest sense and encompasses a variety of antibody structures, including but not limited to monoclonal antibodies, polyclonal antibodies, monospecific antibodies, multispecific antibodies (e.g., bispecific antibodies), full-length antibodies, and antibody fragments (or antigen-binding fragments, or antigen-binding moieties), as long as they exhibit the desired antigen-binding activity. For example, a native IgG antibody is a heterotetrameric protein of about 150,000 Daltons composed of two light chains and two heavy chains linked by a disulfide bond. From the N-terminus to the C-terminus, each heavy chain comprises one variable region (VH, also known as variable heavy domain or heavy chain variable region), followed by three constant domains (CH1, CH2, and CH3). Similarly, from the N-terminus to the C-terminus, each light chain comprises one variable region (VL, also known as variable light domain or light chain variable region), followed by one constant light domain (light chain constant region, CL). The term “bispecific antibody” refers to an antibody (including an antibody or an antigen-binding fragment thereof, such as a single-chain antibody) capable of specifically binding to two different antigens or at least two different epitopes of the same antigen. Bispecific antibodies of various structures have been disclosed in the prior art. The bispecific antibodies can be classified into IgG-like bispecific antibodies and antibody-fragment-type bispecific antibodies according to the integrity of IgG molecules. The bispecific antibodies can be classified into bivalent, trivalent, tetravalent, or higher-valency bispecific antibodies according to the number of the antigen-binding regions. The bispecific antibodies can be classified into symmetric bispecific antibodies and asymmetric bispecific antibodies according to whether their structures are horizontally symmetric or asymmetric.

Fragment-type bispecific antibodies such as Fab fragments lacking Fc fragments are formed by linking two or more Fab fragments in one molecule, and they have relatively low immunogenicity, small molecular weights, and relatively high tumor tissue permeability. Typical antibody structures of this type are, for example, F(ab)2, scFv-Fab, (scFv)2-Fab, and the like. The IgG-like bispecific antibodies (e.g., antibodies having Fc fragments) have large relative molecular weight. The Fc fragments facilitate the purification of the antibodies and increase their solubility and stability, and the Fc portions may further bind to the receptor FcRn and increase the serum half-life of the antibodies. Typical structural models of bispecific antibodies are, for example, KiH, CrossMAb, Triomab quadroma, FcΔΔdp, ART-Ig, BiMAb, Biclonics, BEAT, DuoBody, Azymetric, XmAb, 2:1 TCBs, 1Fab-IgG TDB, FynomAb, two-in-one/DAF, scFv-Fab-IgG, DART-Fc, LP-DART, CODV-Fab-TL, HLE-BiTE, F(ab)2-CrossMAb, IgG-(scFv)2, Bs4Ab, DVD-Ig, Tetravalent-DART-Fc, (scFv)4-Fc, CODV-Ig, mAb2, F(ab)4-CrossMAb, and the like (see Aran F. Labrijn et al., Nature Reviews Drug Discovery, volume 18, pages 585-608 (2019); Chen S1 et al., J Immunol Res., Feb. 11, 2019; 2019:4516041).

The term “variable region” or “variable domain” refers to a domain in an antigen-binding molecule that is involved in antigen binding. Herein, the heavy chain variable region in the antigen-binding moiety that specifically binds to CGRP is denoted by CGRP-VH, and the light chain variable region is denoted by CGRP-VL. The heavy chain variable region in the antigen-binding moiety that specifically binds to PACAP is denoted by PACAP-VH, and the light chain variable region is denoted by PACAP-VL. The VH and VL each comprise four conserved framework regions (FRs) and three complementarity-determining regions (CDRs).

The term “complementarity-determining region” or “CDR” refers to a region within a variable domain that primarily contributes to antigen binding.

“Framework” or “FR” refers to residues in a variable domain other than CDR residues. A VH comprises 3 CDRs: HCDR1, HCDR2, and HCDR3; a VL comprises 3 CDRs: LCDR1, LCDR2, and LCDR3.

Herein, the 3 CDRs in the CGRP-VH are denoted by CGRP-HCDR1, CGRP-HCDR2, and CGRP-HCDR3, respectively; the 3 CDRs in the CGRP-VL are denoted by CGRP-LCDR1, CGRP-LCDR2, and CGRP-LCDR3, respectively.

The 3 CDRs in the PACAP-VH are denoted by PACAP-HCDR1, PACAP-HCDR2, and PACAP-HCDR3, respectively; the 3 CDRs in the PACAP-VL are denoted by PACAP-LCDR1, PACAP-LCDR2, and PACAP-LCDR3, respectively.

Each VH and VL is, from the N-terminus to the C-terminus: FR1, CDR1, FR2, CDR2, FR3, CDR3, FR4.

The amino acid sequence boundaries of the CDRs can be determined by a variety of well-known schemes, for example, the “Kabat” numbering scheme (see Kabat et al. (1991), “Sequences of Proteins of Immunological Interest”, 5th ed., Public Health Service, National Institutes of Health, Bethesda, MD), the “Chothia” numbering scheme, the “ABM” numbering scheme, the “contact” numbering scheme (see Martin, A C R. Protein Sequence and Structure Analysis of Antibody Variable Domains[J]. 2001), and the ImMunoGenTics (IMGT) numbering scheme (Lefranc, M. P. et al., Dev. Comp. Immunol., 27, 55-77 (2003); Front Immunol., Oct. 16, 2018; 9:2278), and the like. The corresponding relationships between the various numbering schemes are well known to those skilled in the art. The numbering schemes of the present disclosure are shown in Table 1 below.

TABLE 1
The relationships between CDR numbering schemes

| CDR | IMGT | Kabat | AbM | Chothia | Contact |
|---|---|---|---|---|---|
| HCDR1 | 27-38 | 31-35 | 26-35 | 26-32 | 30-35 |
| HCDR2 | 56-65 | 50-65 | 50-58 | 52-56 | 47-58 |
| HCDR3 | 105-117 | 95-102 | 95-102 | 95-102 | 93-101 |
| LCDR1 | 27-38 | 24-34 | 24-34 | 24-34 | 30-36 |
| LCDR2 | 56-65 | 50-56 | 50-56 | 50-56 | 46-55 |
| LCDR3 | 105-117 | 89-97 | 89-97 | 89-97 | 89-96 |

Unless otherwise stated, the “Kabat” numbering scheme is applied to the variable region and CDR sequences in examples of the present disclosure. Although one numbering scheme (e.g., Kabat) is employed to define amino acid residues in specific embodiments, corresponding technical solutions for other numbering schemes are to be considered as equivalent technical solutions.

The term “antibody fragment” is used to distinguish it from an intact antibody; it retains the ability of the intact antibody to bind to an antigen. Examples of antibody fragments include, but are not limited to, Fv, Fab, Fab′, Fab′-SH, F(ab′)₂, single-domain antibodies, single-chain Fab (scFab), diabodies, linear antibodies, single-chain antibody molecules (e.g., scFv), and multispecific antibodies formed from antibody fragments.

The term “Fc region” or “fragment crystallizable region” is used to define the C-terminal region of the heavy chain of an antibody, including native Fc regions and engineered Fc regions. In some embodiments, the Fc region comprises two identical or different subunits.

In some embodiments, the Fc region of the human IgG heavy chain is defined as extending from the amino acid residue at position Cys226 or from Pro230 to its carboxyl terminus. Suitable native sequence Fc regions for the antibodies described herein include human IgG1, IgG2 (IgG2A, IgG2B), IgG3, and IgG4. Unless otherwise stated, the numbering scheme for the Fc region is the EU index.

The term “Titin chain” refers to, in a Titin protein, a peptide fragment that is 78-118 amino acids in length and comprises a Titin Ig-like 152 domain, or a functional variant thereof. The Titin chain is capable of binding to the Obscurin Ig-like 1 or Obscurin-like Ig-like 1 domain to form a dimerized complex.

The term “Obscurin chain” refers to, in the Obscurin protein, a peptide fragment that is 87-117 amino acids in length and comprises an Obscurin Ig-like 1 domain, or a functional variant thereof, or to, in the Obscurin-like 1 protein, a peptide fragment that is 78-118 amino acids in length and comprises an Obscurin-like Ig-like 1 domain, or a functional variant thereof. The Obscurin chain is capable of binding to the Titin Ig-like 152 domain to form a dimerized complex.

The Titin chain and the Obscurin chain of the present disclosure can be used to substitute the CH1 and CL in the Fab, respectively, to form a substituted Fab (Fab-S), and the substitution does not affect the binding of the antigen-binding molecule to the antigen or an epitope thereof.

The term “chimeric” antibody refers to an antibody in which a portion of the heavy and/or light chain is derived from a particular source or species, while the remainder of the heavy and/or light chain is derived from a different source or species.

The term “humanized” antibody refers to an antibody that retains the reactivity of a non-human antibody while having low immunogenicity in humans. For example, the humanization can be achieved by retaining the non-human CDRs and replacing the remainder of the antibody with human counterparts (i.e., the constant regions and the framework region portion of the variable regions).

The term “affinity” refers to the overall strength of a non-covalent interaction between a single binding site of a molecule (e.g., an antigen-binding molecule) and its binding partner (e.g., an antigen or epitope). Unless otherwise indicated, as used herein, “binding affinity” refers to an internal binding affinity. The affinity of molecule X for its ligand Y can be generally expressed by the equilibrium dissociation constant (KD). Affinity can be determined by conventional methods known in the art, including those described herein. The term “kassoc” or “ka” refers to the association rate of a particular antibody-antigen interaction, while the term “kdis” or “kd” as used herein refers to the dissociation rate of a particular antibody-antigen interaction. As used herein, the term “KD” refers to the equilibrium dissociation constant, which is the ratio of kd to ka (i.e., kd/ka) and expressed as molar concentration (M). The KD value of an antibody can be measured using methods known in the art, such as a surface plasmon resonance assay, an ELISA, or solution equilibrium titration (SET).

The term “monoclonal antibody” refers to a population of substantially homogeneous antibodies or a member thereof, that is, the amino acid sequences of the antibody molecules comprised in the population are identical, except for a small number of natural mutations that may exist. In contrast, a polyclonal antibody generally comprises a plurality of different antibodies having different amino acid sequences in their variable domains, which are generally specific for different epitopes. “Monoclonal” refers to the characteristics of an antibody obtained from a substantially homogeneous antibody population and should not be construed as requiring the production of the antibody by any particular method. In some embodiments, the antibody provided in the present disclosure is a monoclonal antibody.

The term “antigen” refers to a molecule or a portion of a molecule that can be selectively recognized or bound by an antigen-binding molecule (e.g., an antibody). An antigen may have one or more epitopes capable of interacting with different antigen-binding molecules (e.g., antibodies).

The term “epitope” refers to an area or region on an antigen that is capable of specifically binding to an antibody or an antigen-binding fragment thereof. Epitopes can be formed by contiguous amino acids (linear epitopes) or comprise non-contiguous amino acids (conformational epitopes), e.g., non-contiguous amino acids that are in spatial proximity to each other due to folding of an antigen (i.e., by tertiary folding of an antigen of a protein nature). The difference between the conformational epitope and the linear epitope is that in the presence of denaturing solvents, the binding of the antibody to the conformational epitope is lost. An epitope comprises at least 3, at least 4, at least 5, at least 6, at least 7, or 8-10 amino acids in a unique spatial conformation. Screening for antibodies that bind to particular epitopes (i.e., those that bind to identical epitopes) can be performed using routine methods in the art, such as but not limited to, alanine scanning, peptide blotting (see Meth. Mol. Biol. 248 (2004) 443-463), peptide cleavage analysis, epitope excision, epitope extraction, chemical modification of the antigen (see Prot. Sci. 9 (2000) 487-496), and cross-blocking (see “Antibodies”, Harlow and Lane (Cold Spring Harbor Press, Cold Spring Harb., NY)).

The term “capable of specifically binding”, “specifically binding”, or “binding” means that an antibody is capable of binding to a certain antigen or an epitope thereof with higher affinity than to other antigens or epitopes. Generally, an antibody binds to an antigen or an epitope thereof with an equilibrium dissociation constant (KD) of about 1×10⁻⁷ M or less (e.g., about 1×10⁻⁸ M, 1×10⁻⁹ M, 1×10⁻¹⁰ M, or less). In some embodiments, the KD for the binding of an antibody to an antigen is 10% or less (e.g., 1%) of the KD for the binding of the antibody to a non-specific antigen (e.g., BSA or casein). KD may be determined using known methods, for example, by a FACS or BIACORE® surface plasmon resonance assay. However, an antibody that specifically binds to an antigen or an epitope thereof may have cross-reactivity to other related antigens, e.g., to corresponding antigens from other species (homologous), such as humans or monkeys, e.g., Macaca fascicularis (cynomolgus, cyno), Pan troglodytes (chimpanzee, chimp), or Callithrix jacchus (common marmoset, marmoset).

The term “not bind” means that an antibody is not capable of binding to a certain antigen or an epitope thereof in the specific binding manner described above. For example, when an antibody binds to an antigen or an epitope thereof with an equilibrium dissociation constant (KD) of about 1×10⁻⁶ M or greater, the antibody is considered not to bind to the antigen.

The term “antigen-binding moiety” refers to a polypeptide molecule that specifically binds to an antigen of interest or an epitope thereof. A specific antigen-binding moiety includes an antigen-binding domain of an antibody, e.g., comprises a heavy chain variable region and a light chain variable region.

The term “antigen-binding moiety that specifically binds to CGRP” refers to a moiety that is capable of binding to CGRP or an epitope thereof with sufficient affinity. For example, the antigen-binding moiety that specifically binds to CGRP has the following equilibrium dissociation constant (KD): <about 1 nM, <about 0.1 nM, or <about 0.01 nM, as measured by a surface plasmon resonance assay. Antigen-binding moieties include antibody fragments as defined herein, e.g., a Fab, a substituted Fab, or an scFv.

The term “linker” refers to a linker unit that links two polypeptide fragments. Herein, the linkers present in the same structural formula may be identical or different. The linker may be a peptide linker comprising one or more amino acids, typically about 1-30, 2-24, or 3-15 amino acids. The linkers used herein may be identical or different. When “-” appears in a structural formula, it means that the units on both sides are directly linked by a covalent bond.

“Tm” is the temperature of dissolution and denaturation (endogenous fluorescence). When the protein is denatured (by heating or by a denaturant), the tertiary structure is opened and the aromatic amino acid microenvironment changes, resulting in a change in the emission fluorescence spectrum. In the present disclosure, Tm1 refers to the temperature at which the fluorescence value changes to half of the maximum value.

“Tonset” is the onset temperature of denaturation. It refers to the temperature at which the protein begins to denature, i.e., the temperature at which the fluorescence value begins to change.

“Tagg” is the onset temperature of aggregation. The temperature at which the sample begins to aggregate is monitored by detecting aggregates at two wavelengths of 266 nm and 473 nm by static light scattering. Tagg 266 refers to the onset temperature of aggregation monitored at 266 nm.

The term “nucleic acid” is used interchangeably herein with the term “polynucleotide” and refers to deoxyribonucleotide or ribonucleotide and a polymer thereof in either single-stranded or double-stranded form. The term encompasses nucleic acids comprising known nucleotide analogs or modified backbone residues or linkages, which are synthetic, naturally occurring, and non-naturally occurring, have similar binding properties to the reference nucleic acid, and are metabolized similarly to the reference nucleotide. “Isolated” nucleic acid refers to a nucleic acid molecule that has been separated from components of its natural environment. The isolated nucleic acid includes a nucleic acid molecule comprised in a cell that generally comprises the nucleic acid molecule, but the nucleic acid molecule is present extrachromosomally or at a chromosomal position different from its natural chromosomal position. An isolated nucleic acid encoding the antigen-binding molecule refers to one or more nucleic acid molecules encoding antibody heavy and light chains (or fragments thereof), including such nucleic acid molecule(s) in a single vector or separate vectors and such nucleic acid molecule(s) present at one or more locations in a host cell. Unless otherwise stated, a particular nucleic acid sequence also implicitly encompasses conservatively modified variants thereof (e.g., degenerate codon substitutions) and complementary sequences, as well as the sequence explicitly indicated. Specifically, as detailed below, degenerate codon substitutions may be obtained by generating sequences in which the third position of one or more selected (or all) codons is substituted with mixed bases and/or deoxyinosine residues.

The terms “polypeptide” and “protein” are used interchangeably herein and refer to a polymer of amino acid residues. The terms apply to amino acid polymers in which one or more amino acid residues are artificial chemical mimics of corresponding naturally occurring amino acids, as well as to naturally occurring amino acid polymers and non-naturally occurring amino acid polymers. Unless otherwise stated, a particular polypeptide sequence also implicitly encompasses conservatively modified variants thereof.

The term sequence “identity” refers to the degree (percentage) to which the amino acids/nucleic acids of two sequences are identical at equivalent positions when the two sequences are optimally aligned; in the process of the alignment, gaps are allowed to be introduced as necessary to achieve the maximum percent sequence identity, but any conservative substitution is not considered as part of sequence identity. To determine percent sequence identity, alignments can be accomplished by techniques known to those skilled in the art, for example, using publicly available computer software, such as BLAST, BLAST-2, ALIGN, ALIGN-2, or Megalign (DNASTAR) software. Those skilled in the art can determine parameters suitable for measuring alignment, including any algorithms required to achieve maximum alignment of the full length of the aligned sequences.

The term “fusion” or “linkage” means that components (e.g., antigen-binding moieties and Fc domains) are covalently linked directly or via a linker.

The term “vector” means a polynucleotide molecule capable of transporting another polynucleotide linked thereto. One type of vector is a “plasmid”, which refers to a circular double-stranded DNA loop into which additional DNA segments can be ligated. Another type of vector is a viral vector, such as an adeno-associated viral vector (AAV or AAV2), wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. The term “expression vector” or “expression construct” refers to a vector that is applied to transform a host cell and comprises a nucleic acid sequence that directs and/or controls (along with the host cell) the expression of one or more heterologous coding regions operably linked thereto. Expression constructs may include, but are not limited to, sequences that affect or control transcription and translation and affect RNA splicing of a coding region operably linked thereto in the presence of an intron.

The terms “host cell”, “host cell line”, and “host cell culture” are used interchangeably and refer to cells into which exogenous nucleic acids have been introduced, including progenies of such cells. Host cells include “transformants” and “transformed cells”, which include primary transformed cells and progeny derived therefrom, regardless of the number of passages. Progeny may not be completely identical to parent cells in terms of nucleic acid content and may contain mutations. As used herein, the term includes mutant progeny that have the same function or biological activity as the cells screened or selected from the primary transformed cells. Host cells include prokaryotic and eukaryotic host cells, wherein the eukaryotic host cells include, but are not limited to, mammalian cells, insect cell lines, plant cells, and fungal cells. Mammalian host cells include human, mouse, rat, canine, monkey, porcine, goat, bovine, equine, and hamster cells, including but not limited to, Chinese hamster ovary (CHO) cells, NSO, SP2 cells, HeLa cells, baby hamster kidney (BHK) cells, monkey kidney cells (COS), human hepatocellular carcinoma cells (e.g., Hep G2), A549 cells, 3T3 cells, and HEK-293 cells. Fungal cells include yeast and filamentous fungal cells, including, for example, Pichia pastoris, Pichia finlandica, Pichia trehalophila, Pichia koclamae, Pichia membranaefaciens, Pichia minuta (Ogataea minuta, Pichia lindneri), Pichia opuntiae, Pichia thermotolerans, Pichia salictaria, Pichia guercuum, Pichia pijperi, Pichia stiptis, Pichia methanolica, Pichia, Saccharomycescerevisiae, Saccharomyces, Hansenula polymorpha, Kluyveromyces, Kluyveromyces lactis, Candida albicans, Aspergillus nidulans, Aspergillus niger, Aspergillus oryzae, Trichoderma reesei, Chrysosporium lucknowense, Fusarium sp., Fusarium gramineum, Fusarium venenatum, Physcomitrella patens, and Neurospora crassa. Pichia, any Saccharomyces, Hansenula polymorpha, any Kluyveromyces, Candida albicans, any Aspergillus, Trichoderma reesei, Chrysosporium lucknowense, any Fusarium, Yarrowia lipolytica, and Neurospora crassa. The host cell of the present patent does not include objects not authorized by the Patent Laws. “Optional” or “optionally” means that the feature subsequently described may, but does not necessarily, occur, which indicates that instances in which the feature occurs or does not occur are included.

The term “pharmaceutical composition” refers to a mixture comprising one or more of the antigen-binding molecules or antibodies described herein and other chemical components; the other components are, for example, physiological/pharmaceutically acceptable vehicles and excipients.

The term “pharmaceutically acceptable vehicle” refers to an ingredient in a pharmaceutical formulation that is different from the active ingredient and is not toxic to the subject. Pharmaceutically acceptable vehicles include, but are not limited to, buffers, excipients, stabilizers, or preservatives.

The term “subject” or “individual” includes humans and non-human animals. Non-human animals include all vertebrates (e.g., mammals and non-mammals) such as non-human primates (e.g., cynomolgus monkeys), sheep, dogs, cows, chickens, amphibians, and reptiles. Unless indicated, the terms “patient” and “subject” are used interchangeably herein. As used herein, the term “cynomolgus monkey (cyno)” or “cynomolgus” refers to Macaca fascicularis. In certain embodiments, the individual or subject is a human.

“Administrating” or “giving”, when applied to animals, humans, experimental subjects, cells, tissue, organs, or biological fluids, refers to contact of an exogenous drug, a therapeutic agent, a diagnostic agent, or a composition with the animals, humans, subjects, cells, tissue, organs, or biological fluids.

The term “sample” refers to a collection isolated from a subject (such as fluids, cells, or tissues), as well as fluids, cells, or tissues present in a subject. Exemplary samples are biological fluids (such as blood; serum; serosal fluids; plasma; lymph; urine; saliva; cystic fluids; tears; excretions; sputum; mucosal secretions of secretory tissues or secretory organs; vaginal secretions; ascites; fluids in the pleura; pericardium; peritoneum; fluids from abdominal cavity and other body cavities; fluids collected from bronchial lavage; synovial fluids; liquid solutions in contact with a subject or biological source, e.g., culture media (including conditioned culture media); lavage fluids; and the like), tissue biopsy samples, fine needle punctures, surgically excised tissues, organ cultures, or cell cultures.

“Treatment” or “treat” (and grammatical variations thereof) refers to a clinical intervention intended to change the individual being treated, and the clinical intervention may be performed either for prophylaxis or during the course of clinical pathology. Desirable effects of the treatment include, but are not limited to, preventing the occurrence or recurrence of a disease, palliating symptoms, palliating/diminishing any direct or indirect pathological consequences of the disease, preventing metastasis, decreasing the rate of disease progression, ameliorating or palliating the disease state, and regressing or improving prognosis. In some embodiments, the molecule of the present disclosure is used to delay the development of or slow the progression of disease.

“Effective amount” is generally an amount that is sufficient to reduce the severity and/or frequency of symptoms, eliminate symptoms and/or underlying causes, prevent the appearance of symptoms and/or their underlying causes, and/or ameliorate or improve damage caused by or associated with a disease state.

In some examples, the effective amount is a therapeutically effective amount or a prophylactically effective amount.

“Therapeutically effective amount” is an amount that is sufficient to treat a disease state or symptom, particularly a state or symptom associated with the disease state, or to otherwise prevent, hinder, delay, or reverse the progression of the disease state or any other undesirable symptoms associated with the disease in any way.

“Prophylactically effective amount” is an amount that, when administered to a subject, will have a predetermined prophylactic effect, e.g., preventing or delaying the onset (or recurrence) of the disease state, or reducing the likelihood of the onset (or recurrence) of the disease state or associated symptoms.

A complete therapeutic or prophylactic effect does not necessarily occur after administration of one dose and may occur after administration of a series of doses. Thus, a therapeutically or prophylactically effective amount may be administered in one or more doses. “Therapeutically effective amount” and “prophylactically effective amount” may vary depending on a variety of factors such as the disease state, age, sex, and weight of the individual, and the ability of a therapeutic agent or combination of therapeutic agents to elicit a desired response in the individual. Exemplary indicators of an effective therapeutic agent or combination of therapeutic agents include, for example, improved health condition of a subject.

### Target Molecule

“CGRP” should be understood in the broadest sense and is intended to encompass various forms of molecules of CGRP in various stages in mammals, such as, but not limited to, molecules produced by the CGRP gene during amplification, replication, transcription, splicing, processing, translation, and modification, e.g., precursor BCMA, mature CGRP, CGRP expressed on the membrane, CGRP splice variants, modified CGRP, or fragments thereof; the term also encompasses CGRP artificially prepared or expressed in vitro.

“PACAP” should be understood in the broadest sense and is intended to encompass various forms of molecules of PACAP in various stages in mammals, such as, but not limited to, molecules produced by the PACAP gene during amplification, replication, transcription, splicing, processing, translation, and modification, e.g., precursor PACAP, mature PACAP, PACAP expressed on the membrane, PACAP splice variants, modified PACAP, or fragments thereof; the term also encompasses PACAP artificially prepared or expressed in vitro.

### Antigen-Binding Molecule of the Present Disclosure

The present disclosure provides an antigen-binding molecule having a number of advantageous properties, such as affinity, inhibitory activity against binding of PACAP38 to a receptor, inhibition of cAMP production in cells under conditions of CGRP or PACAP induction, therapeutic activity, safety, pharmacokinetic properties, and druggability (e.g., yield, purity, and stability).

### Exemplary Antigen-Binding Molecules

The antigen-binding molecule of the present disclosure includes bispecific antigen-binding molecules that specifically bind to CGRP and PACAP (e.g., bispecific antibodies), anti-CGRP antibodies, or anti-PACAP antibodies. Particularly, the antigen-binding molecule of the present disclosure has any one of or a combination of the following properties a to d:
a. High affinity for CGRP. In some embodiments, the antigen-binding molecule binds to human CGRP at 25° C. with a KD of less than 1×10⁻¹⁰ M or less than 1×10⁻¹¹ M, as measured by a surface plasmon resonance assay. In some embodiments, the antigen-binding molecule binds to rat CGRP at 25° C. with a KD of less than 1×10⁻⁹ M or less than 1×10⁻¹ M, as measured by a surface plasmon resonance assay. In some embodiments, the antigen-binding molecule binds to human CGRP or rat CGRP with an EC₅₀ of less than 1×10⁻⁸ M, as measured by an ELISA. In some embodiments, the antigen-binding molecule has cross-binding activity to human CGRP and rat CGRP.
b. High affinity and specificity for PACAP. In some embodiments, the antigen-binding molecule binds to PACAP38 and PACAP27 with an EC₅₀ of less than 1×10⁻⁹ M or less than 0.5×10⁻⁹ M, as measured by an ELISA. In some embodiments, the antigen-binding molecule does not bind to VIP.
c. Inhibiting cAMP production in cells under conditions of CGRP induction. In some embodiments, the antigen-binding molecule inhibits cAMP production in cells with an IC₅₀ of less than 4×10⁻⁸ M, less than 1×10⁻⁸ M, or less than 5×10⁻⁹ M under conditions of CGRP induction. In some embodiments, the cells are SK-N-MC cells.
d. Inhibiting cAMP production in cells under conditions of PACAP induction. In some embodiments, the antigen-binding molecule inhibits cAMP production in cells with an IC50 of less than 2×10⁻⁸ M, less than 1×10⁻⁸ M, or less than 5×10⁻⁹ M under conditions of PACAP induction. In some embodiments, the cells are SH-SY5Y cells.

In one aspect, the present disclosure provides an antigen-binding molecule comprising at least one antigen-binding moiety that specifically binds to CGRP and at least one antigen-binding moiety that specifically binds to PACAP, wherein the antigen-binding moiety that specifically binds to CGRP comprises a heavy chain variable region CGRP-VH and a light chain variable region CGRP-VL, and the antigen-binding moiety that specifically binds to PACAP comprises a heavy chain variable region PACAP-VH and a light chain variable region PACAP-VL. In another aspect, the present disclosure provides an antigen-binding molecule that is an anti-CGRP antibody comprising a heavy chain variable region CGRP-VH and a light chain variable region CGRP-VL. In another aspect, the present disclosure provides an antigen-binding molecule that is an anti-PACAP antibody comprising a heavy chain variable region PACAP-VH and a light chain variable region PACAP-VL.
(i) In some embodiments, provided is the aforementioned antigen-binding molecule, wherein the CGRP-VH comprises: a CGRP-HCDR1 whose amino acid sequence is set forth in SEQ ID NO: 22, a CGRP-HCDR2 whose amino acid sequence is set forth in SEQ ID NO: 106, 23, 103, 104, or 105, and a CGRP-HCDR3 whose amino acid sequence is set forth in SEQ ID NO: 24, and the CGRP-VL comprises: a CGRP-LCDR1 whose amino acid sequence is set forth in SEQ ID NO: 25, a CGRP-LCDR2 whose amino acid sequence is set forth in SEQ ID NO: 26, and a CGRP-LCDR3 whose amino acid sequence is set forth in SEQ ID NO: 27.

In some embodiments, provided is the antigen-binding molecule according to any one of the foregoing, wherein the CGRP-VH comprises: a CGRP-HCDR1 whose amino acid sequence is set forth in SEQ ID NO: 22, a CGRP-HCDR2 whose amino acid sequence is set forth in SEQ ID NO: 106, and a CGRP-HCDR3 whose amino acid sequence is set forth in SEQ ID NO: 24, and the CGRP-VL comprises: a CGRP-LCDR1 whose amino acid sequence is set forth in SEQ ID NO: 25, a CGRP-LCDR2 whose amino acid sequence is set forth in SEQ ID NO: 26, and a CGRP-LCDR3 whose amino acid sequence is set forth in SEQ ID NO: 27.

In some embodiments, provided is the antigen-binding molecule according to any one of the foregoing, wherein the CGRP-VH and/or the CGRP-VL are/is murine or humanized.

In some embodiments, the CGRP-VH and/or the CGRP-VL are humanized. In some embodiments, a FR1, a FR2, and a FR3 of the humanized CGRP-VH have at least 60%, 70%, or 80% sequence identity to a FR1, a FR2, and a FR3 of SEQ ID NO: 46, and a FR4 of the humanized CGRP-VH has at least 80% or 90% sequence identity to a FR4 of SEQ ID NO: 46; a FR1, a FR2, and a FR3 of the humanized CGRP-VL have at least 60%, 70%, or 80% sequence identity to a FR1, a FR2, and a FR3 of SEQ ID NO: 47, and/or a FR4 of the humanized CGRP-VL has at least 80% or 90% sequence identity to a FR4 of SEQ ID NO: 47. In some embodiments, the CGRP-VH comprises a FR1, a FR2, and a FR3 derived from IGHV1-3*01 and a FR4 derived from IGHJ6*01, and the framework regions of the heavy chain variable region are unsubstituted or have one or more amino acid substitutions selected from the group consisting of 1E, 44C, 48I, 67A, 69L, 71V, 73K, and 94S; and/or the CGRP-VL comprises a FR1, a FR2, and a FR3 derived from IGKV1-12*01 and a FR4 derived from IGKJ4*01, and the framework regions of the light chain variable region are unsubstituted or have one or more amino acid substitutions selected from the group consisting of 43S, 46A, and 100C. In some embodiments, the variable regions and CDRs described above are defined according to the Kabat numbering scheme.

In some embodiments, provided is the antigen-binding molecule according to any one of the foregoing, wherein the amino acid sequence of the CGRP-VH has at least 90%, 95%, 96%, 97%, 98%, or 99% sequence identity to SEQ ID NO: 83, 46, 76, 77, 78, 79, 80, 81, or 82, and the amino acid sequence of the CGRP-VL has at least 90%, 95%, 96%, 97%, 98%, or 99% sequence identity to SEQ ID NO: 85, 47, or 84. In some embodiments, the amino acid sequence of the CGRP-VH is set forth in SEQ ID NO: 83, 46, 76, 77, 78, 79, 80, 81, or 82, and the amino acid sequence of the CGRP-VL is set forth in SEQ ID NO: 85, 47, or 84. In some embodiments, the amino acid sequence of the CGRP-VH is set forth in SEQ ID NO: 83, 76, 77, 78, 79, 80, 81, or 82, and the amino acid sequence of the CGRP-VL is set forth in SEQ ID NO: 85 or 84.

In some embodiments, provided is the antigen-binding molecule according to any one of the foregoing, wherein:
the amino acid sequence of the CGRP-VH is set forth in SEQ ID NO: 83, and the amino acid sequence of the CGRP-VL is set forth in SEQ ID NO: 85; or
the amino acid sequence of the CGRP-VH is set forth in SEQ ID NO: 46, and the amino acid sequence of the CGRP-VL is set forth in SEQ ID NO: 47; or
the amino acid sequence of the CGRP-VH is set forth in SEQ ID NO: 76, and the amino acid sequence of the CGRP-VL is set forth in SEQ ID NO: 84; or
the amino acid sequence of the CGRP-VH is set forth in SEQ ID NO: 77, and the amino acid sequence of the CGRP-VL is set forth in SEQ ID NO: 84; or
the amino acid sequence of the CGRP-VH is set forth in SEQ ID NO: 78, and the amino acid sequence of the CGRP-VL is set forth in SEQ ID NO: 84; or
the amino acid sequence of the CGRP-VH is set forth in SEQ ID NO: 79, and the amino acid sequence of the CGRP-VL is set forth in SEQ ID NO: 84; or
the amino acid sequence of the CGRP-VH is set forth in SEQ ID NO: 80, and the amino acid sequence of the CGRP-VL is set forth in SEQ ID NO: 84; or
the amino acid sequence of the CGRP-VH is set forth in SEQ ID NO: 81, and the amino acid sequence of the CGRP-VL is set forth in SEQ ID NO: 84; or
the amino acid sequence of the CGRP-VH is set forth in SEQ ID NO: 82, and the amino acid sequence of the CGRP-VL is set forth in SEQ ID NO: 84.

In some embodiments, provided is the antigen-binding molecule according to any one of the foregoing, wherein the amino acid sequence of the CGRP-VH is set forth in SEQ ID NO: 83, and the amino acid sequence of the CGRP-VL is set forth in SEQ ID NO: 85; or
the amino acid sequence of the CGRP-VH is set forth in SEQ ID NO: 81, and the amino acid sequence of the CGRP-VL is set forth in SEQ ID NO: 84.
(ii) In some embodiments, provided is the aforementioned antigen-binding molecule, wherein the CGRP-VH comprises: a CGRP-HCDR1 whose amino acid sequence is set forth in SEQ ID NO: 16, a CGRP-HCDR2 whose amino acid sequence is set forth in SEQ ID NO: 101, 17, 100, 102, or 192, and a CGRP-HCDR3 whose amino acid sequence is set forth in SEQ ID NO: 18, and the CGRP-VL comprises: a CGRP-LCDR1 whose amino acid sequence is set forth in SEQ ID NO: 19, a CGRP-LCDR2 whose amino acid sequence is set forth in SEQ ID NO: 20, and a CGRP-LCDR3 whose amino acid sequence is set forth in SEQ ID NO: 21.

In some embodiments, provided is the aforementioned antigen-binding molecule, wherein the CGRP-VH comprises: a CGRP-HCDR1 whose amino acid sequence is set forth in SEQ ID NO: 16, a CGRP-HCDR2 whose amino acid sequence is set forth in SEQ ID NO: 101, and a CGRP-HCDR3 whose amino acid sequence is set forth in SEQ ID NO: 18, and the CGRP-VL comprises: a CGRP-LCDR1 whose amino acid sequence is set forth in SEQ ID NO: 19, a CGRP-LCDR2 whose amino acid sequence is set forth in SEQ ID NO: 20, and a CGRP-LCDR3 whose amino acid sequence is set forth in SEQ ID NO: 21.

In some embodiments, provided is the antigen-binding molecule according to any one of the foregoing, wherein the CGRP-VH and/or the CGRP-VL are/is murine or humanized.

In some embodiments, the CGRP-VH and/or the CGRP-VL are humanized. In some embodiments, a FR1, a FR2, and a FR3 of the humanized CGRP-VH have at least 60%, 70%, or 80% sequence identity to a FR1, a FR2, and a FR3 of SEQ ID NO: 44, and a FR4 of the humanized CGRP-VH has at least 80% or 90% sequence identity to a FR4 of SEQ ID NO: 44; a FR1, a FR2, and a FR3 of the humanized CGRP-VL have at least 60%, 70%, or 80% sequence identity to a FR1, a FR2, and a FR3 of SEQ ID NO: 45, and/or a FR4 of the humanized CGRP-VL has at least 80% or 90% sequence identity to a FR4 of SEQ ID NO: 45.

In some embodiments, the CGRP-VH comprises a FR1, a FR2, and a FR3 derived from IGHV1-3*01 and a FR4 derived from IGHJ6*01, and the framework regions of the heavy chain variable region are unsubstituted or have one or more amino acid substitutions selected from the group consisting of 1E, 44C, 481, 67A, 69L, 71V, 73K, and 94S; and/or the CGRP-VL comprises a FR1, a FR2, and a FR3 derived from IGKV1-16*01 and a FR4 derived from IGKJ4*01, and the framework regions of the light chain variable region are unsubstituted or have one or more amino acid substitutions selected from the group consisting of 43S, 46A, and 100C. In some embodiments, the variable regions and CDRs described above are defined according to the Kabat numbering scheme.

In some embodiments, provided is the antigen-binding molecule according to any one of the foregoing, wherein the amino acid sequence of the CGRP-VH has at least 90%, 95%, 96%, 97%, 98%, or 99% sequence identity to SEQ ID NO: 72, 44, 68, 69, 70, or 71, and the amino acid sequence of the CGRP-VL has at least 90%, 95%, 96%, 97%, 98%, or 99% sequence identity to SEQ ID NO: 75, 45, 73, or 74. In some embodiments, the amino acid sequence of the CGRP-VH is set forth in SEQ ID NO: 72, 44, 68, 69, 70, or 71, and the amino acid sequence of the CGRP-VL is set forth in SEQ ID NO: 75, 45, 73, or 74. In some embodiments, the amino acid sequence of the CGRP-VH is set forth in SEQ ID NO: 72, 68, 69, 70, or 71, and the amino acid sequence of the CGRP-VL is set forth in SEQ ID NO: 75, 73, or 74.

In some embodiments, provided is the antigen-binding molecule according to any one of the foregoing, wherein:
the amino acid sequence of the CGRP-VH is set forth in SEQ ID NO: 72, and the amino acid sequence of the CGRP-VL is set forth in SEQ ID NO: 75; or
the amino acid sequence of the CGRP-VH is set forth in SEQ ID NO: 44, and the amino acid sequence of the CGRP-VL is set forth in SEQ ID NO: 45; or
the amino acid sequence of the CGRP-VH is set forth in SEQ ID NO: 68, and the amino acid sequence of the CGRP-VL is set forth in SEQ ID NO: 73; or
the amino acid sequence of the CGRP-VH is set forth in SEQ ID NO: 68, and the amino acid sequence of the CGRP-VL is set forth in SEQ ID NO: 74; or
the amino acid sequence of the CGRP-VH is set forth in SEQ ID NO: 69, and the amino acid sequence of the CGRP-VL is set forth in SEQ ID NO: 73; or
the amino acid sequence of the CGRP-VH is set forth in SEQ ID NO: 69, and the amino acid sequence of the CGRP-VL is set forth in SEQ ID NO: 74; or
the amino acid sequence of the CGRP-VH is set forth in SEQ ID NO: 70, and the amino acid sequence of the CGRP-VL is set forth in SEQ ID NO: 73; or
the amino acid sequence of the CGRP-VH is set forth in SEQ ID NO: 71, and the amino acid sequence of the CGRP-VL is set forth in SEQ ID NO: 73; or
the amino acid sequence of the CGRP-VH is set forth in SEQ ID NO: 71, and the amino acid sequence of the CGRP-VL is set forth in SEQ ID NO: 74.

In some embodiments, provided is the antigen-binding molecule according to any one of the foregoing, wherein the amino acid sequence of the CGRP-VH is set forth in SEQ ID NO: 72, and the amino acid sequence of the CGRP-VL is the amino acid sequence set forth in SEQ ID NO: 75.
(iii) The CGRP-VH comprises: a CGRP-HCDR1 whose amino acid sequence is set forth in SEQ ID NO: 4, a CGRP-HCDR2 whose amino acid sequence is set forth in SEQ ID NO: 5, 93, 94, or 95, and a CGRP-HCDR3 whose amino acid sequence is set forth in SEQ ID NO: 6, and the CGRP-VL comprises: a CGRP-LCDR1 whose amino acid sequence is set forth in SEQ ID NO: 7, a CGRP-LCDR2 whose amino acid sequence is set forth in SEQ ID NO: 8, and a CGRP-LCDR3 whose amino acid sequence is set forth in SEQ ID NO: 9.

In some embodiments, provided is the aforementioned antigen-binding molecule, wherein the CGRP-VH and/or the CGRP-VL are/is murine or humanized. In some embodiments, the CGRP-VH and/or the CGRP-VL are humanized. In some embodiments, a FR1, a FR2, and a FR3 of the humanized CGRP-VH have at least 60%, 70%, or 80% sequence identity to a FR1, a FR2, and a FR3 of SEQ ID NO: 40, and a FR4 of the humanized CGRP-VH has at least 80% or 90% sequence identity to a FR4 of SEQ ID NO: 40; a FR1, a FR2, and a FR3 of the humanized CGRP-VL have at least 60%, 70%, or 80% sequence identity to a FR1, a FR2, and a FR3 of SEQ ID NO: 41, and/or a FR4 of the humanized CGRP-VL has at least 80% or 90% sequence identity to a FR4 of SEQ ID NO: 41. In some embodiments, the CGRP-VH comprises a FRI, a FR2, and a FR3 derived from IGHV1-69*02 and a FR4 derived from IGHJ6*01, and the framework regions of the heavy chain variable region are unsubstituted or have one or more amino acid substitutions selected from the group consisting of 1E, 27Y, and 94G; and/or the CGRP-VL comprises a FR1, a FR2, and a FR3 derived from IGKV2-40*01 and a FR4 derived from IGKJ2*01, and the framework regions of the light chain variable region are unsubstituted or have an amino acid substitution of 28S. In some embodiments, the variable regions and CDRs described above are defined according to the Kabat numbering scheme.

In some embodiments, provided is the antigen-binding molecule according to any one of the foregoing, wherein the amino acid sequence of the CGRP-VH has at least 90%, 95%, 96%, 97%, 98%, or 99% sequence identity to SEQ ID NO: 40, 54, 55, or 56, and the amino acid sequence of the CGRP-VL has at least 90%, 95%, 96%, 97%, 98%, or 99% sequence identity to SEQ ID NO: 41, 57, or 58. In some embodiments, the amino acid sequence of the CGRP-VH is set forth in SEQ ID NO: 40, 54, 55, or 56, and the amino acid sequence of the CGRP-VL is set forth in SEQ ID NO: 41, 57, or 58. In some embodiments, the amino acid sequence of the CGRP-VH is set forth in SEQ ID NO: 54, 55, or 56, and the amino acid sequence of the CGRP-VL is set forth in SEQ ID NO: 57 or 58.

In some embodiments, provided is the antigen-binding molecule according to any one of the foregoing, wherein:
the amino acid sequence of the CGRP-VH is set forth in SEQ ID NO: 40, and the amino acid sequence of the CGRP-VL is set forth in SEQ ID NO: 41; or
the amino acid sequence of the CGRP-VH is set forth in SEQ ID NO: 54, and the amino acid sequence of the CGRP-VL is set forth in SEQ ID NO: 57; or
the amino acid sequence of the CGRP-VH is set forth in SEQ ID NO: 55, and the amino acid sequence of the CGRP-VL is set forth in SEQ ID NO: 57; or
the amino acid sequence of the CGRP-VH is set forth in SEQ ID NO: 56, and the amino acid sequence of the CGRP-VL is set forth in SEQ ID NO: 57; or
the amino acid sequence of the CGRP-VH is set forth in SEQ ID NO: 54, and the amino acid sequence of the CGRP-VL is set forth in SEQ ID NO: 58; or
the amino acid sequence of the CGRP-VH is set forth in SEQ ID NO: 56, and the amino acid sequence of the CGRP-VL is set forth in SEQ ID NO: 58.
(iv) The CGRP-VH comprises: a CGRP-HCDR1 whose amino acid sequence is set forth in SEQ ID NO: 10, a CGRP-HCDR2 whose amino acid sequence is set forth in SEQ ID NO: 11, 96, 97, 98, or 99, and a CGRP-HCDR3 whose amino acid sequence is set forth in SEQ ID NO: 12, and the CGRP-VL comprises: a CGRP-LCDR1 whose amino acid sequence is set forth in SEQ ID NO: 13, a CGRP-LCDR2 whose amino acid sequence is set forth in SEQ ID NO: 14, and a CGRP-LCDR3 whose amino acid sequence is set forth in SEQ ID NO: 15.

In some embodiments, provided is the aforementioned antigen-binding molecule, wherein the CGRP-VH and/or the CGRP-VL are/is murine or humanized. In some embodiments, the CGRP-VH and/or the CGRP-VL are humanized. In some embodiments, a FR1, a FR2, and a FR3 of the humanized CGRP-VH have at least 60%, 70%, or 80% sequence identity to a FR1, a FR2, and a FR3 of SEQ ID NO: 42, and a FR4 of the humanized CGRP-VH has at least 80% or 90% sequence identity to a FR4 of SEQ ID NO: 42; a FR1, a FR2, and a FR3 of the humanized CGRP-VL have at least 60%, 70%, or 80% sequence identity to a FR1, a FR2, and a FR3 of SEQ ID NO: 43, and/or a FR4 of the humanized CGRP-VL has at least 80% or 90% sequence identity to a FR4 of SEQ ID NO: 43. In some embodiments, the CGRP-VH comprises a FR1, a FR2, and a FR3 derived from IGHV1-3*01 and a FR4 derived from IGHJ6*01, and the framework regions of the heavy chain variable region are unsubstituted or have one or more amino acid substitutions selected from the group consisting of 1E, 481, 67A, 69L, 71V, 73K, and 94S; and/or the CGRP-VL comprises a FR1, a FR2, and a FR3 derived from IGKV1-27*01 and a FR4 derived from IGKJ2*01, and the framework regions of the light chain variable region are unsubstituted or have one or more amino acid substitutions selected from the group consisting of 43S, 46A, and 87H.

In some embodiments, the variable regions and CDRs described above are defined according to the Kabat numbering scheme.

In some embodiments, provided is the antigen-binding molecule according to any one of the foregoing, wherein the amino acid sequence of the CGRP-VH has at least 90%, 95%, 96%, 97%, 98%, or 99% sequence identity to SEQ ID NO: 42, 59, 60, 61, 62, 63, 64, 65, or 66, and the amino acid sequence of the CGRP-VL has at least 90%, 95%, 96%, 97%, 98%, or 99% sequence identity to SEQ ID NO: 43 or 67. In some embodiments, the amino acid sequence of the CGRP-VH is set forth in SEQ ID NO: 42, 59, 60, 61, 62, 63, 64, 65, or 66, and the amino acid sequence of the CGRP-VL is set forth in SEQ ID NO: 43 or 67.

In some embodiments, the amino acid sequence of the CGRP-VH is set forth in SEQ ID NO: 59, 60, 61, 62, 63, 64, 65, or 66, and the amino acid sequence of the CGRP-VL is set forth in SEQ ID NO: 67.

In some embodiments, provided is the antigen-binding molecule according to any one of the foregoing, wherein:
the amino acid sequence of the CGRP-VH is set forth in SEQ ID NO: 42, and the amino acid sequence of the CGRP-VL is set forth in SEQ ID NO: 43; or
the amino acid sequence of the CGRP-VH is set forth in SEQ ID NO: 59, and the amino acid sequence of the CGRP-VL is set forth in SEQ ID NO: 67; or
the amino acid sequence of the CGRP-VH is set forth in SEQ ID NO: 60, and the amino acid sequence of the CGRP-VL is set forth in SEQ ID NO: 67; or
the amino acid sequence of the CGRP-VH is set forth in SEQ ID NO: 61, and the amino acid sequence of the CGRP-VL is set forth in SEQ ID NO: 67; or
the amino acid sequence of the CGRP-VH is set forth in SEQ ID NO: 62, and the amino acid sequence of the CGRP-VL is set forth in SEQ ID NO: 67; or
the amino acid sequence of the CGRP-VH is set forth in SEQ ID NO: 63, and the amino acid sequence of the CGRP-VL is set forth in SEQ ID NO: 67; or
the amino acid sequence of the CGRP-VH is set forth in SEQ ID NO: 64, and the amino acid sequence of the CGRP-VL is set forth in SEQ ID NO: 67; or
the amino acid sequence of the CGRP-VH is set forth in SEQ ID NO: 65, and the amino acid sequence of the CGRP-VL is set forth in SEQ ID NO: 67; or
the amino acid sequence of the CGRP-VH is set forth in SEQ ID NO: 66, and the amino acid sequence of the CGRP-VL is set forth in SEQ ID NO: 67.
(v) In some embodiments, provided is the aforementioned antigen-binding molecule, wherein the PACAP-VH comprises: a PACAP-HCDR1 whose amino acid sequence is set forth in SEQ ID NO: 28, a PACAP-HCDR2 whose amino acid sequence is set forth in SEQ ID NO: 29, and a PACAP-HCDR3 whose amino acid sequence is set forth in SEQ ID NO: 107 or 30, and the PACAP-VL comprises: a PACAP-LCDR1 whose amino acid sequence is set forth in SEQ ID NO: 31, a PACAP-LCDR2 whose amino acid sequence is set forth in SEQ ID NO: 108 or 32, and a PACAP-LCDR3 whose amino acid sequence is set forth in SEQ ID NO: 33.

In some embodiments, provided is the aforementioned antigen-binding molecule, wherein the PACAP-VH comprises: a PACAP-HCDR1 whose amino acid sequence is set forth in SEQ ID NO: 28, a PACAP-HCDR2 whose amino acid sequence is set forth in SEQ ID NO: 29, and a PACAP-HCDR3 whose amino acid sequence is set forth in SEQ ID NO: 107, and the PACAP-VL comprises: a PACAP-LCDR1 whose amino acid sequence is set forth in SEQ ID NO: 31, a PACAP-LCDR2 whose amino acid sequence is set forth in SEQ ID NO: 108, and a PACAP-LCDR3 whose amino acid sequence is set forth in SEQ ID NO: 33.

In some embodiments, provided is the antigen-binding molecule according to any one of the foregoing, wherein the PACAP-VH and/or the PACAP-VL are/is murine or humanized. In some embodiments, the PACAP-VH and/or the PACAP-VL are humanized. In some embodiments, a FR1, a FR2, and a FR3 of the humanized PACAP-VH have at least 60%, 70%, or 80% sequence identity to a FR1, a FR2, and a FR3 of SEQ ID NO: 48, and a FR4 of the humanized PACAP-VH has at least 80% or 90% sequence identity to a FR4 of SEQ ID NO: 48; a FR1, a FR2, and a FR3 of the humanized PACAP-VL have at least 60%, 70%, or 80% sequence identity to a FR1, a FR2, and a FR3 of SEQ ID NO: 49, and/or a FR4 of the humanized PACAP-VL has at least 80% or 90% sequence identity to a FR4 of SEQ ID NO: 49. In some embodiments, the PACAP-VH comprises a FR1, a FR2, and a FR3 derived from IGHV3-21*01 and a FR4 derived from IGHJ6*01, and the framework regions of the heavy chain variable region are unsubstituted or have one or more amino acid substitutions selected from the group consisting of 13Q, 28A, 30N, and 49A; and/or the PACAP-VL comprises a FR1, a FR2, and a FR3 derived from IGKV4-1*01 and a FR4 derived from IGKJ4*01, and the framework regions of the light chain variable region are unsubstituted or have one or more amino acid substitutions selected from the group consisting of 4L and 58I. In some embodiments, the variable regions and CDRs described above are defined according to the Kabat numbering scheme.

In some embodiments, provided is the antigen-binding molecule according to any one of the foregoing, wherein the amino acid sequence of the PACAP-VH has at least 90%, 95%, 96%, 97%, 98%, or 99% sequence identity to SEQ ID NO: 87, 48, or 86, and the amino acid sequence of the PACAP-VL has at least 90%, 95%, 96%, 97%, 98%, or 99% sequence identity to SEQ ID NO: 90, 49, 88, or 89. In some embodiments, the amino acid sequence of the PACAP-VH is set forth in SEQ ID NO: 87, 48, or 86, and the amino acid sequence of the PACAP-VL is set forth in SEQ ID NO: 90, 49, 88, or 89. In some embodiments, the amino acid sequence of the PACAP-VH is set forth in SEQ ID NO: 87 or 86, and the amino acid sequence of the PACAP-VL is set forth in SEQ ID NO: 90, 88, or 89.

In some embodiments, provided is the antigen-binding molecule according to any one of the foregoing, wherein:
the amino acid sequence of the PACAP-VH is set forth in SEQ ID NO: 87, and the amino acid sequence of the PACAP-VL is set forth in SEQ ID NO: 90; or
the amino acid sequence of the PACAP-VH is set forth in SEQ ID NO: 48, and the amino acid sequence of the PACAP-VL is set forth in SEQ ID NO: 49; or
the amino acid sequence of the PACAP-VH is set forth in SEQ ID NO: 86, and the amino acid sequence of the PACAP-VL is set forth in SEQ ID NO: 88; or
the amino acid sequence of the PACAP-VH is set forth in SEQ ID NO: 86, and the amino acid sequence of the PACAP-VL is set forth in SEQ ID NO: 89.

In some embodiments, provided is the antigen-binding molecule according to any one of the foregoing, wherein the amino acid sequence of the PACAP-VH is set forth in SEQ ID NO: 87, and the amino acid sequence of the PACAP-VL is the amino acid sequence set forth in SEQ ID NO: 90.
(vi) In some embodiments, provided is the antigen-binding molecule according to any one of the foregoing, wherein the PACAP-VH comprises: a PACAP-HCDR1 whose amino acid sequence is set forth in SEQ ID NO: 34, a PACAP-HCDR2 whose amino acid sequence is set forth in SEQ ID NO: 35 or 109, and a PACAP-HCDR3 whose amino acid sequence is set forth in SEQ ID NO: 36, and the PACAP-VL comprises: a PACAP-LCDR1 whose amino acid sequence is set forth in SEQ ID NO: 37, a PACAP-LCDR2 whose amino acid sequence is set forth in SEQ ID NO: 38, and a PACAP-LCDR3 whose amino acid sequence is set forth in SEQ ID NO: 39.

In some embodiments, provided is the antigen-binding molecule according to any one of the foregoing, wherein the PACAP-VH and/or the PACAP-VL are/is murine or humanized. In some embodiments, the PACAP-VH and/or the PACAP-VL are humanized. In some embodiments, a FR1, a FR2, and a FR3 of the humanized PACAP-VH have at least 60%, 70%, or 80% sequence identity to a FR1, a FR2, and a FR3 of SEQ ID NO: 50, and a FR4 of the humanized PACAP-VH has at least 80% or 90% sequence identity to a FR4 of SEQ ID NO: 50; a FR1, a FR2, and a FR3 of the humanized PACAP-VL have at least 60%, 70%, or 80% sequence identity to a FR1, a FR2, and a FR3 of SEQ ID NO: 51, and/or a FR4 of the humanized PACAP-VL has at least 80% or 90% sequence identity to a FR4 of SEQ ID NO: 51. In some embodiments, the PACAP-VH comprises a FR1, a FR2, and a FR3 derived from IGHV1-69-2*01 and a FR4 derived from IGHJ6*01, and the framework regions of the heavy chain variable region are unsubstituted or have one or more amino acid substitutions selected from the group consisting of 24A, 27F, 28N, 291, 30K, 71T, 76N, 93V, and 94F; and/or the PACAP-VL comprises a FR1, a FR2, and a FR3 derived from IGLV7-43*01 and a FR4 derived from IGLJ2*01, and the framework regions of the light chain variable region are unsubstituted or have one or more amino acid substitutions selected from the group consisting of 36V, 44F, 46G, 49G, 57G, and 58A. In some embodiments, the variable regions and CDRs described above are defined according to the Kabat numbering scheme.

In some embodiments, provided is the antigen-binding molecule according to any one of the foregoing, wherein the amino acid sequence of the PACAP-VH has at least 90%, 95%, 96%, 97%, 98%, or 99% sequence identity to SEQ ID NO: 50 or 91, and the amino acid sequence of the PACAP-VL has at least 90%, 95%, 96%, 97%, 98%, or 99% sequence identity to SEQ ID NO: 51 or 92. In some embodiments, the amino acid sequence of the PACAP-VH is set forth in SEQ ID NO: 50 or 91, and the amino acid sequence of the PACAP-VL is set forth in SEQ ID NO: 51 or 92.

In some embodiments, provided is the antigen-binding molecule according to any one of the foregoing, wherein:
the amino acid sequence of the PACAP-VH is set forth in SEQ ID NO: 50, and the amino acid sequence of the PACAP-VL is set forth in SEQ ID NO: 51; or
the amino acid sequence of the PACAP-VH is set forth in SEQ ID NO: 91, and the amino acid sequence of the PACAP-VL is the amino acid sequence set forth in SEQ ID NO: 92.

In some embodiments, provided is the antigen-binding molecule according to any one of the foregoing, wherein:
the CGRP-VH comprises: a CGRP-HCDR1 whose amino acid sequence is set forth in SEQ ID NO: 22, a CGRP-HCDR2 whose amino acid sequence is set forth in SEQ ID NO: 106, and a CGRP-HCDR3 whose amino acid sequence is set forth in SEQ ID NO: 24, and the CGRP-VL comprises: a CGRP-LCDR1 whose amino acid sequence is set forth in SEQ ID NO: 25, a CGRP-LCDR2 whose amino acid sequence is set forth in SEQ ID NO: 26, and a CGRP-LCDR3 whose amino acid sequence is set forth in SEQ ID NO: 27; or
the CGRP-VH comprises: a CGRP-HCDR1 whose amino acid sequence is set forth in SEQ ID NO: 16, a CGRP-HCDR2 whose amino acid sequence is set forth in SEQ ID NO: 101, and a CGRP-HCDR3 whose amino acid sequence is set forth in SEQ ID NO: 18, and the CGRP-VL comprises: a CGRP-LCDR1 whose amino acid sequence is set forth in SEQ ID NO: 19, a CGRP-LCDR2 whose amino acid sequence is set forth in SEQ ID NO: 20, and a CGRP-LCDR3 whose amino acid sequence is set forth in SEQ ID NO: 21; and
the PACAP-VH comprises: a PACAP-HCDR1 whose amino acid sequence is set forth in SEQ ID NO: 28, a PACAP-HCDR2 whose amino acid sequence is set forth in SEQ ID NO: 29, and a PACAP-HCDR3 whose amino acid sequence is set forth in SEQ ID NO: 107, and the PACAP-VL comprises: a PACAP-LCDR1 whose amino acid sequence is set forth in SEQ ID NO: 31, a PACAP-LCDR2 whose amino acid sequence is set forth in SEQ ID NO: 108, and a PACAP-LCDR3 whose amino acid sequence is set forth in SEQ ID NO: 33.

In some embodiments, provided is the antigen-binding molecule according to any one of the foregoing, wherein:
the amino acid sequence of the CGRP-VH is set forth in SEQ ID NO: 83, and the amino acid sequence of the CGRP-VL is set forth in SEQ ID NO: 85; or
the amino acid sequence of the CGRP-VH is set forth in SEQ ID NO: 81, and the amino acid sequence of the CGRP-VL is set forth in SEQ ID NO: 84; or
the amino acid sequence of the CGRP-VH is set forth in SEQ ID NO: 72, and the amino acid sequence of the CGRP-VL is the amino acid sequence set forth in SEQ ID NO: 75; and
the amino acid sequence of the PACAP-VH is set forth in SEQ ID NO: 87, and the amino acid sequence of the PACAP-VL is the amino acid sequence set forth in SEQ ID NO: 90.

### Structure of Antigen-Binding Molecule

The function of the bispecific antigen-binding molecule of the present disclosure is not limited to a particular molecular structure.

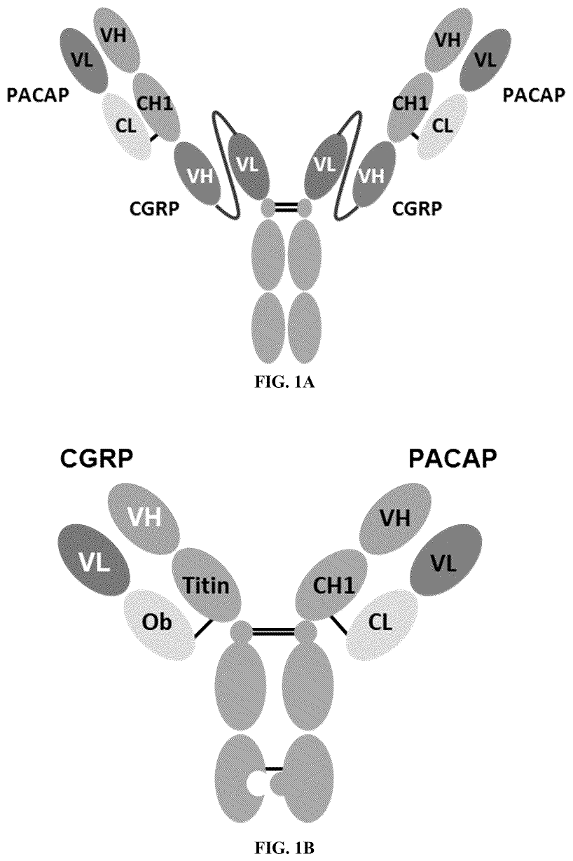

The present disclosure provides a tetravalent (Format 2+2) antigen-binding molecule comprising two antigen-binding moieties that specifically bind to CGRP, two antigen-binding moieties that specifically bind to PACAP, and an Fc region. Illustratively, the antigen-binding molecule comprises two first chains having a structure represented by formula (a) and two second chains having a structure represented by formula (b):

[PACAP-VH]-[CH1]-[CGRP-VH]-[linker 1]-[CGRP-VL]-[linker 2]-[one subunit of the Fc region], and formula (a)

[PACAP-VL]-[CL], formula (b)

wherein the structures represented by formulas (a) and (b) are arranged from the N-terminus to the C-terminus, and the linker 1 and the linker 2 are identical or different peptide linkers; the antigen-binding molecule is schematically shown in FIG. 1A.

The present disclosure further provides a bivalent (Format 1+1) antigen-binding molecule comprising an antigen-binding moiety that specifically binds to CGRP, an antigen-binding moiety that specifically binds to PACAP, and an Fc region. Illustratively, the antigen-binding molecule comprises one first chain having a structure represented by formula (c), one second chain having a structure represented by formula (b), one third chain having a structure represented by formula (d), and one fourth chain having a structure represented by formula (e):

[PACAP-VH]-[CH1]-[Fc1], formula (c)

[PACAP-VL]-[CL], formula (b)

[CGRP-VH]-[linker 3]-[Titin chain]-[Fc2], and formula (d)

[CGRP-VL]-[linker 4]-[Obscurin chain], formula (e)

wherein the structures represented by formulas (c), (b), (d), and (e) are arranged from the N-terminus to the C-terminus, and the linker 3 and the linker 4 are identical or different peptide linkers; the antigen-binding molecule is schematically shown in FIG. 1B.

The peptide linkers can be any suitable peptide chains, as long as the antigen-binding molecule is capable of exhibiting the desired antigen-binding activity. For example, the peptide linkers may be flexible peptides of 1-50 or 3-20 amino acid residues. In some embodiments, the peptide linkers each independently have a structure of L₁-(GGGGS)n-L₂, wherein L₁ is a bond, A, GS, GGS, or GGGS, n is 0, 1, 2, 3, 4, 5, 6, 7, 8, 9, or 10, L2 is a bond, G, GG, GGG, or GGGG, and the peptide linkers are not bonds. In some embodiments, the peptide linkers are 3-15 amino acid residues in length. In some embodiments, the peptide linkers each independently have a structure of (GGGGS)n, wherein n is 1, 2, or 3. In some embodiments, the peptide linkers are GGG (SEQ ID NO: 186), GGGGS (SEQ ID NO: 187), GGGGSGGGGS (SEQ ID NO: 188), or GGGGSGGGGSGGGGS (SEQ ID NO: 189). In some embodiments, the peptide linker 1 is GGGGSGGGGSGGGGS (SEQ ID NO: 189), and the peptide linker 2 is GGG (SEQ ID NO: 186). In some embodiments, both the peptide linker 3 and the peptide linker 4 are GGGGS (SEQ ID NO: 187).

In some embodiments, formula (a) is: [PACAP-VH]-[CH1]-[CGRP-VH]-[GGGGSGGGGSGGGGS]-[CGRP-VL]-[GGG]-[one subunit of the Fc region].

In some embodiments, formula (d) is: [CGRP-VH]-[GGGGS]-[Titin chain]-[Fc2].

In some embodiments, formula (e) is: [CGRP-VL]-[GGGGS]-[Obscurin chain].

An exemplary tetravalent antigen-binding molecule comprises:
two first chains whose amino acid sequences are set forth in SEQ ID NO: 113 and two second chains whose amino acid sequences are set forth in SEQ ID NO: 114; or
the antigen-binding molecule comprises: two first chains comprising the amino acid sequence of SEQ ID NO: 194 and two second chains comprising the amino acid sequence of SEQ ID NO: 114; or
two first chains whose amino acid sequences are set forth in SEQ ID NO: 111 and two second chains whose amino acid sequences are set forth in SEQ ID NO: 112.

An exemplary bivalent antigen-binding molecule comprises a first chain whose amino acid sequence is set forth in SEQ ID NO: 117, a second chain whose amino acid sequence is set forth in SEQ ID NO: 118, a third chain whose amino acid sequence is set forth in SEQ ID NO: 119, and a fourth chain whose amino acid sequence is set forth in SEQ ID NO: 120. The monospecific antigen-binding molecule of the present disclosure may be a chimeric antibody or a humanized antibody.

### Variants of Antigen-Binding Molecule

In certain embodiments, amino acid sequence variants of the antigen-binding molecule provided herein are encompassed. For example, it may be desirable to improve the binding affinity and/or other biological properties of the antibody. Amino acid sequence variants of an antibody can be prepared by introducing appropriate modifications into the nucleotide sequence encoding the antibody, or by peptide synthesis. Such modifications include, for example, deletions from, and/or insertions into and/or substitutions of residues within the amino acid sequence of the antigen-binding molecule. Any combination of deletion, insertion, and substitution can be made to obtain the final construct, provided that the final construct possesses the desired characteristics, e.g., antigen binding.

### Substitutional, Insertional, and Deletional Variants

In certain embodiments, an antigen-binding molecule variant having one or more amino acid substitutions is provided. The substitutions are made at sites of interest, including CDRs and FRs. Conservative substitutions are shown in Table 2 under the heading of “Preferred substitution”. More substantial changes are provided in Table 2 under the heading of “exemplary substitution”, and as further described below with reference to amino acid side chain classes. Amino acid substitutions can be introduced into an antibody of interest and the products are screened for a desired activity, e.g., retained/improved antigen binding, reduced immunogenicity, or improved ADCC or CDC.

TABLE 2
Amino acid substitutions

| Original residue | Exemplary substitution | Preferred substitution |
|---|---|---|
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Ser | Ser |
| Trp (W) | Tyr; Phe | Tyr |
| Tyr (Y) | Trp; Phe; Thr; Ser | Phe |
| Val (V) | Ile; Leu; Met; Phe; Ala; norleucine | Leu |

According to common side-chain properties, amino acids can be grouped as follows:
(1) hydrophobic: norleucine, Met, Ala, Val, Leu, Ile;
(2) neutral, hydrophilic: Cys, Ser, Thr, Asn, Gln;
(3) acidic: Asp, Glu;
(4) basic: His, Lys, Arg;
(5) residues affecting chain orientation: Gly, Pro;
(6) aromatic: Trp, Tyr, Phe.

A non-conservative substitution refers to substituting a member of one class for a member of another class.

One type of substitutional variant involves substituting one or more CDR residues of a parent antibody (e.g., a humanized or human antibody). Generally, the resulting variant selected for further study will have changes (e.g., improvements) in certain biological properties (e.g., increased affinity or reduced immunogenicity) relative to the parent antibody, and/or will have substantially retained certain biological properties of the parent antibody. An exemplary substitutional variant is an affinity-matured antibody, which can be conveniently produced, for example, using phage display-based affinity maturation techniques such as those described herein. Briefly, one or more CDR residues are mutated and the variant antibodies are displayed on phages and screened for a particular biological activity (e.g. binding affinity). Changes (e.g., substitutions) can be made in CDRs, for example, to improve antibody affinity. Such changes can be made in CDR “hotspots”, i.e., residues encoded by codons that undergo mutation at high frequency during the somatic maturation process, and/or antigen-contacting residues, and meanwhile, the resulting variant VH or VL is tested for binding affinity. In some embodiments of affinity maturation, diversity is introduced into the variable genes selected for maturation by any one of a variety of methods (e.g., error-prone PCR, chain shuffling, or oligonucleotide-directed mutagenesis). A secondary library is then created. The library is then screened to identify any antibody variants with the desired affinity. Another method for introducing diversity involves CDR-directed methods in which several CDR residues (e.g., 4-6 residues at a time) are randomized. CDR residues involved in antigen binding can be specifically identified, for example, using alanine scanning mutagenesis or modeling. Particularly, HCDR3 and LCDR3 are often targeted.

In certain embodiments, substitutions, insertions or deletions may occur within one or more CDRs, as long as such changes do not substantially reduce the ability of the antibody to bind to the antigen. For example, conservative changes (e.g., conservative substitutions as provided herein) that do not substantially reduce binding affinity may be made in CDRs. Such changes may be, for example, in regions other than antigen-contacting residues. In certain embodiments of the variant VH and VL sequences provided above, each CDR is unaltered or contains no more than 1, 2, or 3 amino acid substitutions.

A useful method for identifying residues or regions of an antibody that can be used as mutagenesis targets is called “alanine scanning mutagenesis”. In this method, when a residue or residue group (e.g., charged residues such as Arg, Asp, His, Lys, and Glu) is substituted with a neutral or negatively charged amino acid (e.g., Ala or polyalanine), whether the interaction of the antibody with the antigen is affected is determined. Further substitutions may be introduced at amino acid locations that show functional sensitivity to the initial substitutions. In addition, a crystal structure of an antigen-antibody complex may be studied to identify contact points between the antibody and antigen. These contact residues and neighboring residues may be targeted or eliminated as substitution candidates. Variants may be screened to determine whether they contain the desired properties.

Amino acid sequence insertions include: the fusion of a polypeptide that is one residue to 100 or more residues in length at the amino-terminus and/or the carboxyl-terminus, and the intrasequence insertion of single or multiple amino acid residues. Examples of terminal insertions include: an antibody with an N-terminal methionyl residue. Other insertional variants include a fusion in which an enzyme or a polypeptide that extends the serum half-life of an antibody is fused to the N- or C-terminus of the antibody.

### Engineering of Fab

In one aspect, in the antigen-binding molecule of the present disclosure, one of the antigen-binding moiety that specifically binds to CGRP and the antigen-binding moiety that specifically binds to PACAP is a substituted Fab, and the substituted Fab comprises a heavy chain variable region, a light chain variable region, a Titin chain, and an Obscurin chain. In the substituted Fab, the original CH1 and CL of the Fab are substituted with the Titin chain and the Obscurin chain. Illustratively, the sequences of the Titin chain and the Obscurin chain are shown in Tables 3-1 and 3-2.

TABLE 3-1
The amino acid sequences of Titin chains

| | SEQ ID | |
|---|---|---|
| No. | NO | Amino acid sequence |
| T.0 | 121 | GIPPKIEALPSDISIDEGKVLTVACAFTGEPTPEVTWSCGGRKIHSQEQGRFHIE |
| | | NTDDLTTLIIMDVQKQDGGLYTLSLGNEFGSDSATVNIHIRSI |
| T.1 | 122 | GIPPKIECLPSDISIDEGKVLTVASAFTGEPTPEVTWSTGGRKIHSQEQGRFHIE |
| | | NTDDLTTLIIMDVQKQDGGLYTLSLGNEFGSDSATVNIHIRSI |
| T.2 | 123 | GIPPKIEALPSDISIDEGKCLTVASAFTGEPTPEVTWSTGGRKIHSQEQGRFHIE |
| | | NTDDLTTLIIMDVQKQDGGLYTLSLGNEFGSDSATVNIHIRSI |
| T.3 | 124 | GIPPKIEALPSDISIDEGKVLTVASCFTGEPTPEVTWSTGGRKIHSQEQGRFHIE |
| | | NTDDLTTLIIMDVQKQDGGLYTLSLGNEFGSDSATVNIHIRSI |
| T.4 | 125 | GIPPKIEALPSDISIDEGKVLCVASAFTGEPTPEVTWSTGGRKIHSQEQGRFHIE |
| | | NTDDLTTLIIMDVQKQDGGLYTLSLGNEFGSDSATVNIHIRSI |
| T.5 | 126 | KAGIRGIPPKIECLPSDISIDEGKVLTVASAFTGEPTPEVTWSTGGRKIHSQEQG |
| | | RFHIENTDDLTTLIIMDVQKQDGGLYTLSLGNEFGSDSATVNIHIRSI |
| T.6 | 127 | KAGIRGIPPKIEALPSDISIDEGKVLTVACAFTGEPTPEVTWSCGGRKIHSQEQ |
| | | GRFHIENTDDLTTLIIMDVQKQDGGLYTLSLGNEFGSDSATVNIHIRSI |
| T.7 | 128 | GIPPKIECLPSDISIDEGKVLTVASAFTGEPTPEVTWSTGGRKIHSQEQGRFHIE |
| | | NTDDLTTLIISDVQKQDGGLYSLSLGNEFGSDSATVNIHIRSI |
| T.8 | 129 | GIPPKIECLPSDISIDEGKVLTVASAFTGEPTPEVTWSTGGRKIHSQEQGRFHIE |
| | | NTDDLTTLIIKDVQRQDGGLYTLTLRNEFGSDSATVNIHIRSI |
| T.9 | 130 | GIWPKIECLPIDLSIDEGKVLMVASAFTGEPTPEVTWSTGGRKIHSQEQGRFHI |
| | | ENTDDLTTLIIMDVQKQDGGLYTLSLGMEFGSDSATVNIHIRSI |
| T.10 | 131 | GIPPKIECLPIDISIDEGKVLTVASAFTGEPTPEVTWSTGGRKIHSQEQGRFHIE |
| | | NTDDLTTLIIKDVQKQDGGLYTLTLRNEFGSDSATVNIHIRSI |
| T.11 | 132 | GIPPKIECLPSDISIDEGKVLTVASAFTGEPTPEVTWSTSGKKISSEEGGRFHIES |
| | | TEDLTTLIIMDVQKQDGGVYTLSLGNDLGSDSATVNIHIRSI |
| T.12 | 133 | GIPPKIECLPSDISIDEGKVLTVASAFTGEPTPEVTWSTGGRKIHSEEGGRFHIE |
| | | STEDLTTLIIMDVQKQDGGVYTLSLGNEFGSDSATVNIHIRSI |
| T.13 | 134 | GIPPKIECLPSDISIDEGKVLTVASAFTGEPTPEVTWSTGGRKIHSQEQGRFHIE |
| | | STEDLTTLIIMDVQKQDGGVYTLSLGNEFGSDSATVNIHIRSI |
| T.14 | 135 | GIPPKIECLPSDISIDEGKVLTVASAFTGEPTPEVTWSTGGRKIHSQEQGRFHIE |
| | | STEDLTTLIISDVQKQDGGLYSLSLGNEFGSDSATVNIHIRSI |
| T.15 | 136 | KAGIRGIPPKIECLPSDISIDEGKVLTVASAFTGEPTPEVTWSTGGRKIHSQEQG |
| | | RFHIESTEDLTTLIISDVQKQDGGLYSLSLGNEFGSDSATVNIHIRSI |
| T.16 | 137 | GIPPKIECLPIDISIDEGKVLTVASAFTGEPTPEVTWSTGGRKIHSQEQGRFHIE |
| | | NTDDSTTLTIKDVQKQDGGLYTLTLRNEFGSDSATVNIHIRSI |
| T.17 | 138 | GIPPKIECLPIDISIDEGKCLTVASAFTGEPTPEVTWSTGGRKIHSQEQGRFHIEN |
| | | TDDSTTLTIKDVQKQDGGLYTLTLRNEFGSDSATVNIHIRSI |
| T.18 | 139 | GIPPKIECLPIDISIDEGKVLTVASCFTGEPTPEVTWSTGGRKIHSQEQGRFHIEN |
| | | TDDSTTLTIKDVQKQDGGLYTLTLRNEFGSDSATVNIHIRSI |

TABLE 3-2
The amino acid sequences of Obscurin chains

| | SEQ ID | |
|---|---|---|
| No. | NO | Amino acid sequence |
| O.0 | 140 | SGAPRFLTRPKAFVVSVGKDATLSCQIVGNPTPQVSWEKDQQPVAAGARFRL |
| | | AQDGDLYRLTILDLALGDSGQYVCRARNAIGEAFAAVGLQVDAEA |
| OL.0 | 141 | QGSPPCFLRFPRPVRVVSGAEAELKCVVLGEPPPVVVWEKGGQQLAASERLS |
| | | FPADGAEHGLLLTAALPTDAGVYVCRARNAAGEAYAAAAVTVLEPP |
| O.1 | 142 | SGAPRFLTRPKAFVVSVGKDATLSCQIVGNPTPQVSWEKDQQPVAAGARFRL |
| | | AQDGDLYRLTILDLALGDSGQYVCRARNAIGEAFACVGLQVDAEA |
| O.2 | 143 | SGCPRFLTRPKAFVVSVGKDATLSCQIVGNPTPQVSWEKDQQPVAAGARFRL |
| | | AQDGDLYRLTILDLALGDSGQYVCRARNAIGEAFAAVGLQVDAEA |
| O.3 | 144 | SGAPRFLTCPKAFVVSVGKDATLSCQIVGNPTPQVSWEKDQQPVAAGARFRL |
| | | AQDGDLYRLTILDLALGDSGQYVCRARNAIGEAFAAVGLQVDAEA |
| O.4 | 145 | SGAPRFLTRPKAFVVSVGKDATLSSQIVGNPTPQVSWEKDQQPVAAGARFRL |
| | | AQDGDLYRLTILDLALGDSGQYVSRARNAIGEAFACVGLQVDAEA |
| O.5 | 146 | SGCPRFLTRPKAFVVSVGKDATLSSQIVGNPTPQVSWEKDQQPVAAGARFRL |
| | | AQDGDLYRLTILDLALGDSGQYVSRARNAIGEAFAAVGLQVDAEA |
| O.6 | 147 | SGAPRFLTCPKAFVVSVGKDATLSSQIVGNPTPQVSWEKDQQPVAAGARFRL |
| | | AQDGDLYRLTILDLALGDSGQYVSRARNAIGEAFAAVGLQVDAEA |
| O.7 | 148 | SGAPRFKTRPKAFVVSVGKDATLSSQIVGNPTPQVSWEKDQQPVAAGARFRL |
| | | AQDGDLYRLKILDLALGDSGQYVSRARNAIGEAFACVGLQVDAEA |
| O.8 | 149 | SGAPRFKTRPKAFVVSVGKDATLSSQIVGNPTPQVSWEKDQQPVAAGARFRL |
| | | AQDGDLYRLHILDLALGDSGQYVSRARNAIGEAFACVGLQVDAEA |
| O.9 | 150 | SGAPRFLTRPLAFVVSVGKDATLSSQIVGNPTPQVSWEKDQQPVAAGARFRL |
| | | AQDGDLYRLKILDLALGDSGQYVSRARNAIGEAFACVGLQVDAEA |
| O.10 | 151 | SGAPRFLTRPLAFVVSVGKDATLSSQIVGNPTPQVSWEKDQQPVAAGARFRL |
| | | AQDGDLYRLHILDLALGDSGQYVSRARNAIGEAFACVGLQVDAEA |
| O.11 | 152 | DQPQFSGAPRFLTRPKAFVVSVGKDATLSSQIVGNPTPQVSWEKDQQPVAAG |
| | | ARFRLAQDGDLYRLTILDLALGDSGQYVSRARNAIGEAFACVGLQVDAEA |
| O.12 | 153 | DQPQFSGAPRFKTRPKAFVVSVGKDATLSSQIVGNPTPQVSWEKDQQPVAAG |
| | | ARFRLAQDGDLYRLKILDLALGDSGQYVSRARNAIGEAFACVGLQVDAEA |
| O.13 | 154 | DQPQFSGAPRFKTRPKAFVVSVGKDATLSSQIVGNPTPQVSWEKDQQPVAAG |
| | | ARFRLAQDGDLYRLHILDLALGDSGQYVSRARNAIGEAFACVGLQVDAEA |
| O.14 | 155 | DQPQFSGAPRFRTRPKAFVVSVGKDATLSSQIVGNPTPQVSWEKDQQPVAAG |
| | | ARFRLAQDGDLYRLKILDLALGDSGQYVSRARNAIGEAFACVGLQVDAEA |
| O.15 | 156 | DQPQFSGAPRFRTRPKAFVVSVGKDATLSSQIVGNPTPQVSWEKDQQPVAAG |
| | | ARFRLAQDGDLYRLHILDLALGDSGQYVSRARNAIGEAFACVGLQVDAEA |
| O.16 | 157 | DQPQFSGAPRFLTRPKAFVVSVGKDATLSCQIVGNPTPQVSWEKDQQPVAAG |
| | | ARFRLAQDGDLYRLTILDLALGDSGQYVCRARNAIGEAFAAVGLQVDAEA |
| OL.1 | 158 | QGSPPEFLRFPRPVRVVSGAEAELKCVVLGEPPPVVVWEKGGQQLAASERLS |
| | | FPADGAEHGLLLTAALPTDAGCYVCRARNAAGEAYAAAAVTVLEPP |
| OL.2 | 159 | QGSPPEFLRFPRPVRVVSGAEAELKCVVLGEPPPVVVWEKGGQQLAASERLS |
| | | FPADGAEHGLLLTAALPTDAGVYVCRARNAACEAYAAAAVTVLEPP |
| OL.3 | 160 | QGSPPEFLRFPRPVRVVSGAEAELKCVVLGEPPPVVVWEKGGQQLAASERLS |
| | | FPADGAEHGLLLTAALPTDAGVYVCRARNAAGECYAAAAVTVLEPP |
| OL.4 | 161 | QGSPPEFLRFPRPVRVVSGAEAELKSVVLGEPPPVVVWEKGGQQLAASERLS |
| | | FPADGAEHGLLLTAALPTDAGCYVSRARNAAGEAYAAAAVTVLEPP |
| OL.5 | 162 | QGSPPEFLRFPRPVRVVSGAEAELKSVVLGEPPPVVVWEKGGQQLAASERLS |
| | | FPADGAEHGLLLTAALPTDAGVYVSRARNAACEAYAAAAVTVLEPP |
| OL.6 | 163 | QGSPPEFLRFPRPVRVVSGAEAELKSVVLGEPPPVVVWEKGGQQLAASERLS |
| | | FPADGAEHGLLLTAALPTDAGVYVSRARNAAGECYAAAAVTVLEPP |
| O.17 | 164 | SGAPRFLTRPKSYTVSVGKDASLSSQIVGNPTPQVSWEKDQQPVAAGARFRL |
| | | AQDGDLYRLKILDLALGDSGQYVSRARNAIGEAFACVGLQVDAEA |
| O.18 | 165 | SEAPRFLTRPLAFVVSEGKDATLSSQIVGDPPPEVTWEKDQQPITAGARFRLA |
| | | QDGDVYRLEILDLQLSDSGQYVSRARNAIGEAFACVGLQVDAEG |
| O.19 | 166 | SGAPRFLTRPLAFVVSVGKLAMLSSQIVGNPTPQVSWEKDQQPVAAGARFRL |
| | | LQDGDLYRLKILDLALGDSGQYVSRARNAIGEAFACVGLQVDAEA |
| O.20 | 167 | SGAPRFLTRPLAFVVSVGKDATLSSQIVGNPTPQVSWEKDKQPVTAGARFRL |
| | | AQDGDLYRLKILDLQLSDSGQYVSRARNAIGEAFACLGLQVDAEA |
| O.21 | 168 | SGAPRFLTRPLAFVVSVGKDATLSSQIVGNPTPQVSWEKDQLPVTAGARFRL |
| | | AQDGDLYRLKILDLTLSDSGQYVSRARNAIGEAFACVGLEVGAEA |
| O.22 | 169 | SGAPRFLTRPLSYVVSVGKDASLSSQIVGNPTPQVSWEKDQLPVTAGARFRL |
| | | AQDGDLYRLKILDLQLSDSGQYVSRARNAIGEAFACVGLEVGAEA |
| O.23 | 170 | DQPQFSGAPRFLTRPLSYVVSVGKDASLSSQIVGNPTPQVSWEKDQLPVTAG |
| | | ARFRLAQDGDLYRLKILDLQLSDSGQYVSRARNAIGEAFACVGLEVGAEA |
| O.24 | 171 | SGAPRFLTRPKAFVVSVGKDATLSSQIVGNPFPQVSWEKDKQPVTAGVRFRL |
| | | AQDGDLYRLKILDLQLSDSGQYVSRARNAIGEAFACLGLQVDAEA |
| O.25 | 172 | SGAPRFLTRPKASVVSVGKDATLSSQIVGNPFPQVSWEKDKQPVTAGVRFRL |
| | | AQDGDLYRLKILDLQLSDSGQYVSRARNAHGEAFACLGLQVDAEA |
| O.26 | 173 | SGCPRFLTRPKASVVSVGKDATLSSQIVGNPFPQVSWEKDKQPVTAGVRFRL |
| | | AQDGDLYRLKILDLQLSDSGQYVSRARNAHGEAFACLGLQVDAEA |
| O.27 | 174 | SGAPRFLTCPKASVVSVGKDATLSSQIVGNPFPQVSWEKDKQPVTAGVRFRL |
| | | AQDGDLYRLKILDLQLSDSGQYVSRARNAHGEAFACLGLQVDAEA |
| O.28 | 175 | SGAPRFLTRPKASVVSVGKDATLSCQIVGNPFPQVSWEKDKQPVTAGVRFRL |
| | | AQDGDLYRLKILDLQLSDSGQYVCRARNAHGEAFACLGLQVDAEA |
| O.29 | 176 | SGCPRFLTRPKASVVSVGKDATLSCQIVGNPFPQVSWEKDKQPVTAGVRFRL |
| | | AQDGDLYRLKILDLQLSDSGQYVCRARNAHGEAFACLGLQVDAEA |
| O.30 | 177 | SGAPRFLTCPKASVVSVGKDATLSCQIVGNPFPQVSWEKDKQPVTAGVRFRL |
| | | AQDGDLYRLKILDLQLSDSGQYVCRARNAHGEAFACLGLQVDAEA |
| O.31 | 178 | SGAPRFLTRPKASVVSVGKDATLSSQIVGNPFPQVSWEKDKQPVTAGVRFRL |
| | | AQDGDLYRLKILDCQLSDSGQYVSRARNAHGEAFACLGLQCDAEA |
| O.32 | 179 | SGCPRFLTRPKASVVSVGKDATLSSQIVGNPFPQVSWEKDKQPVTAGVRFRL |
| | | AQDGDLYRLKILDCQLSDSGQYVSRARNAHGEAFACLGLQCDAEA |
| O.33 | 180 | SGAPRFLTCPKASVVSVGKDATLSSQIVGNPFPQVSWEKDKQPVTAGVRFRL |
| | | AQDGDLYRLKILDCQLSDSGQYVSRARNAHGEAFACLGLQCDAEA |

Engineering of Fc region

In one aspect, the Fc region of the antigen-binding molecule of the present disclosure comprises one or more amino acid substitutions. The one or more amino acid substitutions reduce binding of the antigen-binding molecule to an Fc receptor, e.g., its binding to an Fc7 receptor, and reduce or eliminate effector functions. A natural IgG Fc region, specifically an IgG₁ Fc region or an IgG₄ Fc region, may cause the antigen-binding molecule of the present disclosure to target cells expressing an Fc receptor, rather than cells expressing an antigen. The engineered Fc region of the present disclosure exhibits a reduced binding affinity for an Fc receptor and/or reduced effector functions. In some embodiments, the binding affinity of the engineered Fc region for an Fc receptor is reduced by 50%, 80%, 90%, or 95% or more compared to that of a native Fc region. In some embodiments, the Fc receptor is an Fc7 receptor. In some embodiments, the Fc receptor is a human Fc7 receptor, e.g., FcγRI, FcγRIIa, FcγRIIB, orFcγRIIIa. In some embodiments, the engineered Fc region also has a reduced binding affinity for a complement such as C1q compared to a native Fc region. In some embodiments, the engineered Fc region has no reduced binding affinity for a neonatal Fc receptor (FcRn) compared to a native Fc region. In some examples, the engineered Fc region has reduced effector functions, which may include, but are not limited to, one or more of the following: reduced complement-dependent cytotoxicity (CDC), reduced antibody-dependent cell-mediated cytotoxicity (ADCC), reduced antibody-dependent cellular phagocytosis (ADCP), reduced cytokine secretion, reduced immune complex-mediated antigen uptake by antigen-presenting cells, reduced binding to NK cells, reduced binding to macrophages, reduced binding to monocytes, reduced binding to polymorphonuclear cells, reduced direct signaling-induced apoptosis, reduced dendritic cell maturation, and reduced T cell priming. For the IgG₁ Fc region, amino acid residue substitutions at positions such as positions 238, 265, 269, 270, 297, 327, and 329 can reduce effector functions. In some embodiments, the Fc region is a human IgG₁ Fc region, and the amino acid residues at positions 234 and 235 are A, as numbered according to the EU index. For the IgG₄ Fc region, amino acid residue substitutions at positions such as position 228 can reduce effector functions.

The antigen-binding molecule may also comprise disulfide bond engineering, such as 354C in the first subunit and 349C in the second subunit. To increase the serum half-life of the antigen-binding molecule, 252Y, 254T, and 256E mutations may be introduced.

When the different antigen-binding moieties of the antigen-binding molecule are fused to the two subunits of the Fc region, respectively, it may result in undesired homodimerization. To improve yield and purity, it is thus advantageous to introduce modifications that promote heterodimerization in the Fc region of the antigen-binding molecule of the present disclosure. In some embodiments, the Fc region of the present disclosure comprises engineering according to the knob-into-hole (KIH) technique, which involves introducing a knob structure at the interface of the first subunit and a hole structure at the interface of the second subunit. As such, the knob structure can be positioned in the hole structure, thereby promoting the formation of heterodimers and inhibiting the production of homodimers. The knob structure is constructed by substituting a small amino acid side chain at the interface of the first subunit with a larger side chain (e.g., tyrosine or tryptophan). The hole structure is created at the interface of the second subunit by substituting a large amino acid side chain with a smaller amino acid side chain (e.g., alanine or threonine). The knob structure and the hole structure are prepared by altering the nucleic acid encoding the polypeptide. Optional amino acid substitutions are shown in the table below:

TABLE 4
Combinations of KIH mutations

| | | | | | | | | |
|---|---|---|---|---|---|---|---|---|
| First | T366Y | T366W | T394W | F405W | T366W | T366Y | T366W | F405W |
| subunit | | | | | | F405A | F405W | Y407A |
| Second | Y407T | Y407A | F405A | T394S | T366S | T394W | T394W | T366W |
| subunit | | | | | L368A | Y407T | Y407A | T394S |
| | | | | | Y407V | | | |

In addition to the knob-into-hole technique, other techniques for modifying the CH3 domain of the heavy chain to achieve heterodimerization are also known in the art, e.g., WO96/27011, WO98/050431, EP1870459, WO2007/110205, WO 007/147901, WO2009/089004, WO2010/129304, WO2011/90754, WO2011/143545, WO2012/058768, WO2013/157954, and WO 013/096291.

The C-terminus of the Fc region may be an intact C-terminus (ending with amino acid residues PGK) or a truncated C-terminus, e.g., a truncated C-terminus in which one or two C-terminal amino acid residues have been removed. In a preferred aspect, the C-terminus of the heavy chain is a truncated C-terminus ending with PG. Thus, in some embodiments, intact antibodies may include antibodies with K447 residues and/or G446+K447 residues removed. In some embodiments, intact antibodies may include antibodies without K447 residues and/or G446+K447 residues removed. In some embodiments, intact antibodies have an antibody population of an antibody mixture with and without K447 residues and/or G446+K447 residues.

### Recombination Method

The antigen-binding molecule may be produced using recombination methods. For these methods, one or more isolated nucleic acids encoding the antigen-binding molecule are provided.

In the case of a native antibody, a native antibody fragment, or a bispecific antibody with homodimeric heavy chains, two nucleic acids are required, one for the light chain or a fragment thereof and the other for the heavy chain or a fragment thereof. Such nucleic acids encode an amino acid sequence comprising the VL of the antibody and/or an amino acid sequence comprising the VH of the antibody (e.g., the light chain and/or heavy chain of the antibody). These nucleic acids can be on the same expression vector or different expression vectors.

In the case of a bispecific antibody with heterodimeric heavy chains, four nucleic acids are required, one for the first light chain, one for the first heavy chain comprising the first heteromonomeric Fc-region polypeptide, one for the second light chain, and one for the second heavy chain comprising the second heteromonomeric Fc-region polypeptide. The four nucleic acids can be contained in one or more nucleic acid molecules or expression vectors. Generally, these nucleic acids are located on two or three expression vectors; that is, one vector can comprise more than one of these nucleic acids.

In one embodiment, the present disclosure provides an isolated nucleic acid encoding the aforementioned antibody. Such nucleic acids may each independently encode any one of the aforementioned polypeptide chains. In another aspect, the present disclosure provides one or more vectors (e.g., expression vectors) comprising such nucleic acids. In another aspect, the present disclosure provides a host cell comprising such nucleic acids. In one embodiment, provided is a method for preparing an antigen-binding molecule, wherein the method comprises culturing a host cell comprising a nucleic acid encoding the antibody, as provided above, under conditions suitable for antibody expression, and optionally collecting the antibody from the host cell (or host cell culture medium).

For recombinant production of the antigen-binding molecule, the nucleic acid encoding the protein is isolated and inserted into one or more vectors for further cloning and/or expression in a host cell. Such nucleic acids can be readily isolated and sequenced using conventional procedures (e.g., by using oligonucleotide probes that are capable of specifically binding to genes encoding the heavy and light chains of an antibody), or produced by recombinant methods or obtained by chemical synthesis.

Suitable host cells for cloning or expressing a vector encoding the antibody include prokaryotic or eukaryotic cells described herein. For example, the antibody can be produced in bacteria, particularly when the antibody does not require glycosylation and Fc effector functions. After expression, the antibody can be isolated from the bacterial cell paste in a soluble fraction and can be further purified.

In addition to prokaryotes, eukaryotic microorganisms such as filamentous fungi or yeast are suitable cloning or expression hosts for antibody-encoding vectors, including fungal and yeast strains, whose glycosylation pathways have been “humanized”, such that an antibody with a partially or fully human glycosylation pattern is produced. Suitable host cells for expression of (glycosylated) antibodies may also be derived from multicellular organisms (invertebrates and vertebrates); examples of invertebrate cells include plant and insect cells. A number of baculovirus strains have been identified, which can be used in combination with insect cells, particularly for transfection of Spodoptera frugiperda cells; plant cell cultures may also be used as hosts, see, e.g., U.S. Pat. Nos. 5,959,177, 6,040,498, 6,420,548, 7,125,978, and 6,417,429; vertebrate cells can also be used as hosts, e.g., mammalian cell lines adapted for growth in a suspension. Other examples of suitable mammalian host cell lines include an SV40-transformed monkey kidney CVl line (COS-7); a human embryonic kidney line (293 or 293T cells); baby hamster kidney cells (BHK); mouse Sertoli cells (TM4 cells); monkey kidney cells (CV1); African green monkey kidney cells (VERO-76); human cervical cancer cells (HELA); canine kidney cells (MDCK); buffalo rat liver cells (BRL3A); human lung cells (W138); human liver cells (Hep G2); mouse mammary tumor cells (MMT 060562); TRI cells; MRC 5 cells; and FS4 cells. Other suitable mammalian host cell lines include Chinese hamster ovary (CHO) cells, including DHFR-CHO cells; and myeloma cell lines such as Y0, NS0, and Sp2/0. For reviews of certain mammalian host cell lines suitable for the production of the antibody, see, e.g., Yazaki, P. and Wu, A. M., Methods in Molecular Biology, Vol. 248, Lo, B. K. C. (eds.), Humana Press, Totowa, NJ (2004), pp. 255-268.

### Diagnostic and Therapeutic Compositions

In certain embodiments, the antigen-binding molecule provided in the present disclosure can be used to detect the presence of CGRP and/or PACAP in a biological sample. As used herein, the term “detect” encompasses quantitative or qualitative detection. In certain embodiments, the biological sample includes a cell or tissue, such as tumor tissue.

In one embodiment, provided is an antigen-binding molecule for use in a diagnostic or detection method. In yet another aspect, provided is a method for detecting the presence of CGRP and/or PACAP in a biological sample. In certain embodiments, the method comprises contacting the biological sample with an antigen-binding molecule under suitable conditions and detecting whether a complex is formed between the detection agent and the antigen. Such methods may be in vitro or in vivo methods. In one embodiment, an antigen-binding molecule is used to select a subject suitable for treatment; for example, CGRP and/or PACAP are/is biomarkers/a biomarker for the selection of a subject.

An exemplary disorder that can be diagnosed using the antigen-binding molecule of the present disclosure is, for example, headache or migraine.

In certain embodiments, provided is a labeled antigen-binding molecule. Labels include, but are not limited to, labels or moieties that are detected directly (such as fluorescent, chromophoric, electron-dense, chemiluminescent, and radioactive labels), and moieties that are detected indirectly (e.g., moieties that are detected indirectly through an enzymatic reaction or molecular interaction, such as enzymes or ligands).

In an additional aspect, provided is a pharmaceutical composition comprising the antigen-binding molecule, for example, for use in any of the following treatment methods. In one aspect, the pharmaceutical composition comprises any of the antigen-binding molecules provided herein and a pharmaceutically acceptable vehicle. In another aspect, the pharmaceutical composition comprises any of the antigen-binding molecules provided herein and at least one additional therapeutic agent.

The pharmaceutical composition of the antigen-binding molecule described in the present disclosure is prepared by: mixing such an antigen-binding molecule having desired purity with one or more optional pharmaceutically acceptable vehicles. The pharmaceutical composition is in the form of a freeze-dried composition or an aqueous solution. Formulations for in vivo administration are generally sterile. Sterility can be readily achieved, for example, by filtration through a sterile filter membrane.

### Treatment Method and Route of Administration

Any of the antigen-binding molecules provided herein can be used in the treatment method. In yet another aspect, the present disclosure provides use of the antigen-binding molecule in the manufacture or preparation of a medicament. In one embodiment, the medicament is used for treating headache or migraine. In addition, the medicament is present in an amount effective for the diseases described above. In some embodiments, the effective amount is a unit daily dose or a unit weekly dose. In one such embodiment, the use further comprises administering to a subject an effective amount of at least one additional therapeutic agent (e.g., one, two, three, four, five, or six additional therapeutic agents).

The “subject” according to any of the above embodiments may be a human.

In a specific embodiment, the subject is an individual who has had, is suspected of having, or is susceptible to headache or migraine.

In yet another aspect, provided is a pharmaceutical composition comprising the antigen-binding molecule, e.g., for use in any of the above pharmaceutical uses or treatment methods. In another embodiment, the pharmaceutical composition further comprises at least one additional therapeutic agent.

The antigen-binding molecule of the present disclosure may be used alone or in combination with other agents for treatment. For example, the antigen-binding molecule of the present disclosure may be co-administered with at least one additional therapeutic agent.

The antigen-binding molecule of the present disclosure (and any additional therapeutic agents) may be administered by any suitable means, including parenteral, intrapulmonary, and intranasal administration, and if required for local treatment, intralesional administration. Parenteral infusion includes intramuscular, intravenous, intra-arterial, intraperitoneal, or subcutaneous administration. Administration may be performed via any suitable route, e.g., by injection, such as intravenous or subcutaneous injection, depending in part on whether the administration is short-term or long-term. Various administration schedules are contemplated herein, including but not limited to, single administration or multiple administrations at multiple time points, bolus administration, and pulse infusion. The antigen-binding molecule of the present disclosure will be formulated, administered, and applied in a manner consistent with Good Medical Practice. Factors considered in this context include the specific disorder being treated, the specific mammal being treated, the clinical state of the individual subject, the cause of the disorder, the site of delivery of the agent, the method of administration, the timing of administration, and other factors known to medical practitioners. The antigen-binding molecule needs not be, but is optionally, formulated along with one or more agents currently used for preventing or treating the disorder. The effective amount of such additional agents depends on the amount of the antigen-binding molecule present in the pharmaceutical composition, the type of the disorder or treatment, and other factors discussed above. These are generally used in the same dose and with routes of administration as described herein, or in about 1 to 99% of the dose described herein, or in any dose and by any route that is empirically/clinically determined to be appropriate.

For the prevention or treatment of disease, the appropriate dosage of the antigen-binding molecule of the present disclosure (when used alone or in combination with one or more other additional therapeutic agents) will depend on the type of the disease to be treated, the type of the therapeutic molecule, the severity and course of the disease, whether the therapeutic molecule is administered for prophylactic or therapeutic purposes, previous therapy, the subject's clinical history and response to the therapeutic molecule, and the discretion of the attending physician. The therapeutic molecule is suitably administered to a subject in one or a series of treatments. Depending on the type and severity of the disease, about 1 μg/kg to 15 mg/kg of the antigen-binding molecule can be an initial candidate dose for administration to the subject, whether, for example, by one or more separate administrations, or by continuous infusion. One typical daily dose may range from about 1 μg/kg to 100 mg/kg or more, depending on the factors mentioned above. Accordingly, taking 50 kg body weight as an example, an exemplary unit daily dose is 50 μg-5 g.

### Product

In another aspect of the present disclosure, provided is a product (e.g., a medication box or a kit) comprising the antigen-binding molecule of the present disclosure and optional materials that can be used for treating, preventing, and/or diagnosing the above disorders. The product comprises one or more containers and a label or package insert on or associated with the containers. Suitable containers include, for example, bottles, vials, syringes, IV solution bags, and the like. The containers may be formed of a variety of materials such as glass or plastic. The containers contain the antigen-binding molecule of the present disclosure alone, or the antigen-binding molecule of the present disclosure and another composition for treating, preventing, and/or diagnosing a disease. The containers may have a sterile access port (for example, the containers may be intravenous solution bags or vials). At least one active agent in the composition is the antigen-binding molecule of the present disclosure. The label or package insert indicates that the composition is used to treat the selected condition.

Moreover, the product may comprise:
(a) a first container containing a composition, wherein the composition comprises the antigen-binding molecule of the present disclosure; and
(b) a second container containing a composition, wherein the composition comprises an additional therapeutic agent.

Alternatively or additionally, the product may further comprise a second (or third) container containing a pharmaceutically acceptable buffer. From a commercial and user standpoint, it may further contain other materials as required, including other buffers, diluents, filters, needles, and syringes.

### EXAMPLES AND TEST EXAMPLES

The present disclosure is further described below with reference to the following examples and test examples, which, however, do not limit the present disclosure. The experimental methods in the examples and test examples of the present disclosure in which specific conditions are not specified are generally performed under conventional conditions such as Antibodies: A Laboratory Manual and Molecular Cloning: A Laboratory Manual by Cold Spring Harbor Laboratory, or under conditions recommended by the manufacturers of the starting materials or commercial products. Reagents without specific origins indicated were commercially available conventional reagents.

### Example 1. Antigen-Binding Molecules Comprising Titin Chain/Obscurin Chain

The Titin/Obscurin chains of the present disclosure may be derived from any suitable polypeptide, including polypeptides from WO2021139758, which is incorporated herein by reference in its entirety, and CN202110527339.7 and the patents which refer to it as a priority document, which are incorporated herein by reference in their entirety. Bispecific antibodies were constructed. The CL was the kappa light chain constant region in WO2021139758. The amino acid sequences of the Titin and Obscurin chains are shown in Tables 3-1 and 3-2. The linker sequences included GGGGS (SEQ ID NO: 187), ASTKG (SEQ ID NO: 190), or RTVAS (SEQ ID NO: 191). The amino acid sequences of the Fc1, Fc2, and CH1 in this example are shown below.

>Fc1
(knob, SEQ ID NO: 181)
DKTHTCPPCPAPEAAGGPSVFLFPPKPKDTLMISRTPEVTCVVVDVSHE
DPEVKFNWYVDGVEVHNAKTKPREEQYNSTYRVVSVLTVLHQDWLNGKE
YKCKVSNKALPAPIEKTISKAKGQPREPQVYTLPPCREEMTKNQVSLWC
LVKGFYPSDIAVEWESNGQPENNYKTTPPVLDSDGSFFLYSKLTVDKSR
WQQGNVFSCSVMHEALHNHYTQKSLSLSPGK;
>Fc2
(hole, SEQ ID NO: 182)
DKTHTCPPCPAPEAAGGPSVFLFPPKPKDTLMISRTPEVTCVVVDVSHE
DPEVKFNWYVDGVEVHNAKTKPREEQYNSTYRVVSVLTVLHQDWLNGKE
YKCKVSNKALPAPIEKTISKAKGQPREPQVCTLPPSREEMTKNQVSLSC
AVKGFYPSDIAVEWESNGQPENNYKTTPPVLDSDGSFFLVSKLTVDKS
RWQQGNVFSCSVMHEALHNHYTQKSLSLSPGK;
>CH1
(SEQ ID NO: 183)
ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKV
EPKSC.

### 1.1. DI Bispecific Antibodies

DI-2 to DI-20, DI bispecific antibodies against hNGF and hRANKL comprising a first heavy chain, a second heavy chain, a first light chain, and a second light chain as described below, were constructed with reference to Example 5 in WO2021139758:
the first heavy chain: from the N-terminus to the C-terminus, [VH1-I]-[linker 1]-[Obscurin chain]-[Fc2];
the first light chain: from the N-terminus to the C-terminus, [VL1-I]-[linker 2]-[Titin chain];
the second heavy chain: from the N-terminus to the C-terminus, [VH2-D]-[CH1]-[Fc1]; and
the second light chain: from the N-terminus to the C-terminus, [VL2-D]-[CL].

The VH1-I and VL1-I were the heavy chain variable region and light chain variable region of the IO in WO2021139758, respectively, and the VH2-D and VL2-D were the heavy chain variable region and light chain variable region of the DO in WO2021139758, respectively. The structures of the Obscurin chain, the Titin chain, the linker 1, and the linker 2 in the DI bispecific antibodies of this example are shown in the table below.

TABLE 5
The Obscurin chain/Titin chain and linkers in DI
bispecific antibodies

| | First heavy chain | | First light chain | |
|---|---|---|---|---|
| | Obscurin | Linker | Titin | Linker |
| No. | chain | 1 | chain | 2 |
| DI-2 | O.20 | GGGGS | T.10 | GGGGS |
| DI-3 | O.25 | GGGGS | T.16 | GGGGS |
| DI-4 | O.26 | GGGGS | T.17 | GGGGS |
| DI-5 | O.27 | GGGGS | T.18 | GGGGS |
| DI-6 | O.28 | GGGGS | T.16 | GGGGS |
| DI-7 | O.29 | GGGGS | T.17 | GGGGS |
| DI-8 | O.30 | GGGGS | T.18 | GGGGS |
| DI-9 | O.31 | GGGGS | T.16 | GGGGS |
| DI-10 | O.32 | GGGGS | T.17 | GGGGS |
| DI-11 | O.33 | GGGGS | T.18 | GGGGS |
| DI-12 | O.25 | ASTKG | T.16 | RTVAS |
| DI-13 | O.26 | ASTKG | T.17 | RTVAS |
| DI-14 | O.27 | ASTKG | T.18 | RTVAS |
| DI-15 | O.28 | ASTKG | T.16 | RTVAS |
| DI-16 | O.29 | ASTKG | T.17 | RTVAS |
| DI-17 | O.30 | ASTKG | T.18 | RTVAS |
| DI-18 | O.31 | ASTKG | T.16 | RTVAS |
| DI-19 | O.32 | ASTKG | T.17 | RTVAS |
| DI-20 | O.33 | ASTKG | T.18 | RTVAS |

Note:
The numbering of the Titin and Obscurin chains in the table is shown in Tables 3-1 and 3-2.

The binding activity of bispecific antibodies DI-2 to DI-20 to their antigens was measured using the method in Test Example 4 of WO2021139758. The thermal stability of the antibodies was studied. Study method: The antibodies were diluted with PBS to a concentration of 5 mg/mL, and their thermal stability was measured using a high throughput differential scanning fluorimeter (UNCHAINED; model: Unit). The experimental results show that there were no significant changes in the binding activity of the engineered bispecific antibodies to the antigens; in addition, DI-4 to DI-8, DI-10 to DI-16, and DI-20 exhibited significantly improved Tm1 (° C.) and Tonset (° C.) as compared to DI-2, indicating that the bispecific antibodies have superior thermal stability.

TABLE 6
The binding activity of DI bispecific antibodies

| | |
|---|---|
| RANKL | NGF |

| | |
|---|---|
| No. | EC50 (nM) |

| | | |
|---|---|---|
| DI-2 | 0.3832 | 6.633 |
| DI-3 | 0.3613 | 5.7730 |
| DI-4 | 0.3959 | 6.2930 |
| DI-5 | 0.3290 | 6.1890 |
| DI-6 | 0.2509 | 5.6720 |
| DI-7 | 0.2557 | 6.6430 |
| DI-8 | 0.3643 | 7.6250 |
| DI-9 | 0.2944 | 8.4950 |
| DI-10 | 0.3460 | 7.1660 |
| DI-11 | 0.3721 | 10.9600 |
| DI-12 | 0.4125 | 6.5156 |
| DI-13 | 0.4440 | 5.5420 |
| DI-14 | 0.4182 | 3.2610 |
| DI-15 | 0.2206 | 5.2800 |
| DI-16 | 0.1474 | 5.5140 |
| DI-17 | 0.2329 | 6.7270 |
| DI-18 | 0.2662 | 5.9080 |
| DI-19 | 0.1843 | 5.9280 |
| DI-20 | 0.3184 | 6.4250 |

TABLE 7
The thermal stability of DI bispecific antibodies

| No. | Tm1 (° C.) | Tonset (° C.) |
|---|---|---|
| DI-2 | 55.6 | 48.3 |
| DI-4 | 60.1 | 52.493 |
| DI-5 | 61 | 51.967 |
| DI-6 | 60.8 | 53.012 |
| DI-7 | 60.34 | 52.003 |
| DI-8 | 60.61 | 50.425 |
| DI-10 | 60.2 | 52.766 |
| DI-11 | 57.35 | — |
| DI-12 | 59.9 | 51.726 |
| DI-13 | 61 | 50.988 |
| DI-14 | 61.2 | 52.191 |
| DI-15 | 60.41 | 50.558 |
| DI-16 | 61.5 | 50.691 |
| DI-20 | 60.7 | 51.859 |

Solutions of the DI bispecific antibodies were prepared using a buffer containing 10 mM acetic acid and 9 sucrose and having a pH of 5.5, and the solutions were incubated in an incubator at 40° C. for four weeks. Following the incubation, the antibody solutions were concentrated to the concentration at the start of the incubation, and the solutions were observed for precipitate formation. The experimental results show that there was precipitate formation in the solution of the DI-2 bispecific antibody group, and that DI-3 to DI-7 were more stable than DI-2.

TABLE 8
Precipitate formation in solutions of DI bispecific antibodies

| | | Concentration | |
|---|---|---|---|
| | | to which | |
| | | solution was | Precipitate |
| | Starting | concentrated at | formation |
| No. | concentration | week 4 | in solution |
| DI-2 | 20 mg/mL | 20 mg/mL | Precipitation occurred |
| DI-3 | 20 mg/mL | 20 mg/mL | No precipitation |
| DI-4 | 60 mg/mL | 60 mg/mL | No precipitation |
| DI-5 | 25 mg/mL | 25 mg/mL | No precipitation |
| DI-6 | 60 mg/mL | 60 mg/mL | No precipitation |
| DI-7 | 16 mg/mL | 16 mg/mL | No precipitation |

### 1.2. PL Bispecific Antibodies

PL-1 to PL-19, PL bispecific antibodies against hPDL1 and hCTLA4 comprising a first heavy chain, a second heavy chain, a first light chain, and a second light chain as described below, were constructed:
the first heavy chain: from the N-terminus to the C-terminus, [VH1-P]-[linker 1]-[Obscurin chain]-[Fc1];
the first light chain: from the N-terminus to the C-terminus, [VL1-P]-[linker 2]-[Titin chain];
the second heavy chain: from the N-terminus to the C-terminus, [VH2-L]-[CH1]-[Fc2]; and
the second light chain: from the N-terminus to the C-terminus, [VL2-L]-[CL].

The VH1-P and VL1-P were the heavy chain variable region and light chain variable region of the antibody h1831K in WO2020177733A1, respectively. The amino acid sequences of the VH2-L and VL2-L are shown below.

>VH2-L
(SEQ ID NO: 184)
QVQLVESGGGVVQPGRSLRLSCAASGFTFSSYTMHWVRQAPGKGLEWVT
FISYDGNNKYYADSVKGRFTISRDNSKNTLYLQMNSLRAEDTAIYYCAR
TGWLGPFDYWGQGTLVTVSS;
>VL2-L
(SEQ ID NO: 185)
EIVLTQSPGTLSLSPGERATLSCRASQSVGSSYLAWYQQKPGQAPRLLI
YGAFSRATGIPDRFSGSGSGTDFTLTISRLEPEDFAVYYCQQYGSSPWT
FGQGTKVEIK.

The structures of the Obscurin chain, the Titin chain, the linker 1, and the linker 2 in the PL bispecific antibodies of this example are shown in the table below.

TABLE 9
The Obscurin chain/Titin chain and linkers in PL bispecific antibodies

| | First heavy chain | | First light chain | |
|---|---|---|---|---|
| No. | Obscurin chain | Linker 1 | Titin chain | Linker 2 |
| PL-1 | O.20 | GGGGS | T.10 | GGGGS |
| PL-2 | O.25 | GGGGS | T.16 | GGGGS |
| PL-3 | O.26 | GGGGS | T.17 | GGGGS |
| PL-4 | O.27 | GGGGS | T.18 | GGGGS |
| PL-5 | O.28 | GGGGS | T.16 | GGGGS |
| PL-6 | O.29 | GGGGS | T.17 | GGGGS |
| PL-7 | O.30 | GGGGS | T.18 | GGGGS |
| PL-8 | O.31 | GGGGS | T.16 | GGGGS |
| PL-9 | O.32 | GGGGS | T.17 | GGGGS |
| PL-10 | O.33 | GGGGS | T.18 | GGGGS |
| PL-11 | O.25 | ASTKG | T.16 | RTVAS |
| PL-12 | O.26 | ASTKG | T.17 | RTVAS |
| PL-13 | O.27 | ASTKG | T.18 | RTVAS |
| PL-14 | O.28 | ASTKG | T.16 | RTVAS |
| PL-15 | O.29 | ASTKG | T.17 | RTVAS |
| PL-16 | O.30 | ASTKG | T.18 | RTVAS |
| PL-17 | O.31 | ASTKG | T.16 | RTVAS |
| PL-18 | O.32 | ASTKG | T.17 | RTVAS |
| PL-19 | O.33 | ASTKG | T.18 | RTVAS |

Note:
The numbering of the Titin and Obscurin chains in the table is shown in Tables 3-1 and 3-2.

The binding activity of the PL bispecific antibodies was measured with reference to the ELISA method in Test Example 4 of WO2021139758; the antigens hPDL1 and hCTLA4 were purchased from Sino biology. The thermal stability of the antibodies was studied. Method: The antibodies were diluted with PBS to a concentration of 1.4-3 mg/mL, and their thermal stability was measured using a high throughput differential scanning fluorimeter (UNCHAINED; model: Unit). The experimental results show that the PL bispecific antibodies still exhibited good binding activity to the antigens; in addition, PL-2 to PL-19 exhibited significantly improved Tm1 (° C.), Tagg 266 (° C.), and Tonset (° C.) as compared to PL-1, indicating that the bispecific antibodies have superior thermal stability.

TABLE 10
The binding activity of PL bispecific antibodies

| | |
|---|---|
| hPDL1 | hCTLA4 |

| | |
|---|---|
| No. | EC50 (nM) |

| | | |
|---|---|---|
| PL-1 | 1.6 | 16.5 |
| PL-2 | 0.5 | 8.0 |
| PL-3 | 0.6 | 7.9 |
| PL-4 | 0.7 | 6.5 |
| PL-5 | 0.7 | 6.7 |
| PL-6 | 0.8 | 5.3 |
| PL-7 | 0.5 | 7.3 |
| PL-8 | 1.4 | 14.5 |
| PL-9 | 0.6 | 5.5 |
| PL-10 | 0.6 | 6.9 |
| PL-11 | 0.6 | 5.6 |
| PL-12 | 0.5 | 8.0 |
| PL-13 | 0.5 | 4.4 |
| PL-14 | 1.3 | 6.0 |
| PL-15 | 1.4 | 3.2 |
| PL-16 | 1.7 | 34.9 |
| PL-17 | 1.4 | 6.2 |
| PL-18 | 1.6 | 6.7 |
| PL-19 | 1.6 | 6.5 |

TABLE 11
The thermal stability of PL
bispecific antibodies

| | | Tagg | |
|---|---|---|---|
| | Tm1 | 266 | Tonset |
| No. | (° C.) | (° C.) | (° C.) |
| PL-1 | 61.64 | 64.82 | 52.566 |
| PL-2 | 66.20 | 66.55 | 58.317 |
| PL-3 | 64.07 | 68.28 | 57.661 |
| PL-4 | 70.71 | 67.29 | 56.246 |
| PL-5 | 74.56 | 68.11 | 58.407 |
| PL-6 | 70.43 | 70.12 | 61.069 |
| PL-7 | 68.46 | 67.41 | 63.031 |
| PL-8 | 65.00 | — | — |
| PL-9 | 69.24 | 67.83 | 58.597 |
| PL-10 | 69.63 | 68.12 | 56.788 |
| PL-11 | 65.88 | — | 57.976 |
| PL-12 | 65.54 | 67.94 | — |
| PL-13 | 71.85 | 68.17 | 58.581 |
| PL-14 | 74.18 | 69.42 | 58.589 |
| PL-15 | 70.96 | 69.91 | 58.622 |
| PL-16 | 63.48 | 68.98 | 58.702 |
| PL-17 | 70.15 | 69.86 | 56.193 |
| PL-18 | — | 69.43 | — |
| PL-19 | — | 69.52 | 57.766 |

### 1.3. HJ Bispecific Antibodies

HJ-3 to HJ11, HJ bispecific antibodies against hIL 5 and hTSLP comprising a first heavy chain, a second heavy chain, a first light chain, and a second light chain as described below, were constructed:
the first heavy chain: from the N-terminus to the C-terminus, [VH1-H]-[linker 1]-[Titin chain]-[Fc1];
the first light chain: from the N-terminus to the C-terminus, [VL1-H]-[linker 2]-[Obscurin chain];
the second heavy chain: from the N-terminus to the C-terminus, [VH12-J]-[CH1]-[Fc2]; and the second light chain: from the N-terminus to the C-terminus, [VL2-J]-[CL].

The VH1-H and VL1-I were the heavy chain variable region and light chain variable region of the H0 in WO2021139758, respectively, and the VH2-J and VL2-J were the heavy chain variable region and light chain variable region of the J1 in WO2021139758, respectively. The structures of the Obscurin chain, the Titin chain, the linker 1, and the linker 2 in the HJ bispecific antibodies of this example are shown in the table below.

TABLE 12
The Obscurin chain/Titin chain and linkers in HJ bispecific antibodies

| | First heavy chain | | First light chain | |
|---|---|---|---|---|
| No. | Titin chain | Linker 1 | Obscurin chain | Linker 2 |
| HJ-3 | T.10 | GGGGS | O.20 | GGGGS |
| HJ-5 | T.16 | GGGGS | O.25 | GGGGS |
| HJ-6 | T.16 | GGGGS | O.27 | GGGGS |
| HJ-7 | T.16 | GGGGS | O.28 | GGGGS |
| HJ-8 | T.16 | ASTKG | O.25 | RTVAS |
| HJ-9 | T.16 | ASTKG | O.27 | RTVAS |
| HJ-10 | T.16 | ASTKG | O.28 | RTVAS |
| HJ-11 | T.16 | ASTKG | O.29 | RTVAS |

The antigen-binding activity of the HJ bispecific antibodies was measured with reference to the method in Test Example 4 of WO2021139758. The thermal stability of the antibodies was studied. Method: Diluted solutions of the HJ bispecific antibodies were prepared using a buffer containing 10 mM acetic acid and 9% sucrose and having a pH of 5.5 and then concentrated by ultrafiltration to obtain HJ bispecific antibody solutions with different concentrations (the concentrations of the HJ bispecific antibodies are shown in Table 13-2). Subsequently, the concentrated solutions were incubated in an incubator at 40° C. On day 0 (i.e., before the 40° C. incubation, DO), day 7 (the 7th day of the 40° C. incubation, D7), day 14 (the 14th day of the 40° C. incubation, D14), day 21 (the 21st day of the 40° C. incubation, D21), and day 28 (the 28th day of the 40° C. incubation, D28), samples were taken and their SEC purity was tested. Immediately after 28 days of incubation at 40° C., samples were taken and their CE-SDS purity was tested. The experimental results show that there were no significant changes in the binding activity of the HJ bispecific antibodies constructed in the present disclosure to the antigens; in addition, bispecific antibodies HJ-5 to HJ-11 were superior in thermal stability to HJ-3.

TABLE 13-1
The binding activity of HJ bispecific antibodies

| | |
|---|---|
| hIL5 | hTSLP |

| | |
|---|---|
| No. | EC50 (nM) |

| | | |
|---|---|---|
| HJ-3 | 17.38 | 3.234 |
| HJ-5 | 15.96 | 2.639 |
| HJ-6 | 37.05 | 2.884 |
| HJ-7 | 17.69 | 2.182 |
| HJ-8 | 18.74 | 3.332 |
| HJ-9 | 10.86 | 3.184 |
| HJ-10 | 20.81 | 3.173 |
| HJ-11 | 9.464 | 3.005 |

TABLE 13-2
The accelerated stability of HJ bispecific antibodies

| | | | | | D28 non- |
|---|---|---|---|---|---|
| | D0 | D0 | D28 | D28Δ | reduced CE- |
| | concentration | SEC purity | SEC purity | SEC purity | SDS purity |
| No. | (mg/mL) | (%) | (%) | (%) | (%) |
| HJ-3 | 50 | 98 | 84 | 14 | 67 |
| HJ-5 | 60.8 | 98.09 | 93.86 | 4.23 | 82.93 |
| HJ-6 | 54 | 98.14 | 89.68 | 8.46 | 80.76 |
| HJ-7 | 56.8 | 97.7 | 92.72 | 4.98 | 85.47 |
| HJ-8 | 62.6 | 93.39 | 84.28 | 9.11 | 73.03 |
| HJ-9 | 53.5 | 90.01 | 85.93 | 4.08 | 80.32 |
| HJ-10 | 69.8 | 90.9 | 88.31 | 2.59 | 80.4 |
| HJ-11 | 50.4 | 92.55 | 90.89 | 1.66 | 82.96 |

### Example 2. Expression of Receptors PAC1, VPAC1, and VPAC2 of PACAP

Sequences encoding extracellular regions of the receptors PAC1, VPAC1, and VPAC2 of human PACAP containing an IgG1-Fc tag were inserted into a phr vector to construct expression plasmids, and HEK293 cells were then transfected with the expression plasmids. The specific transfection steps were as follows: HEK293E cells were seeded at a density of 0.8×10⁶ cells/mL in a FreeStyle™ expression culture medium (containing 1% FBS) and cultured on a thermostatic shaker (120 rpm) at 37° C. for 24 h. A plasmid and PEI were well mixed and left to stand for 15 min. The mixture of the plasmid and PEI was added slowly to 200 mL of HEK293E cells, and the cells were cultured on a shaker (8% CO₂, 120 rpm, 37° C.). On day 3 of transfection, a 10% volume of culture medium was added. On day 6 of transfection, the culture system was centrifuged, and the cell supernatant was collected.

>PAC1 extracellular region-Fc
(SEQ ID NO: 1)
MHSDCIFKKEQAMCLEKIQRANELMGFNDSSPGCPGMWDNITCWKPAHV
GEMVLVSCPELFRIFNPDQVWETETIGESDFGDSNSLDLSDMGVVSRNC
TEDGWSEPFPHYFDACGFDEYESETGDQDYYYLSVKADIEGRMDEPKSS
DKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKE
YKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSR
WQQGNVFSCSVMHEALHNHYTQKSLSLSPGK;
>VPAC1 extracellular region-Fc
(SEQ ID NO: 2)
ARLQEECDYVQMIEVQHKQCLEEAQLENETIGCSKMWDNLTCWPATPRG
QVVVLACPLIFKLFSSIQGRNVSRSCTDEGWTHLEPGPYPIACGLDDKA
ASLDEQQTMFYGSVDIEGRMDEPKSSDKTHTCPPCPAPELLGGPSVFLF
PPKPKDTLMISRTPEVTCVVVDVSHEDPEVKFNWYVDGVEVHNAKTKPR
EEQYNSTYRVVSVLTVLHQDWLNGKEYKCKVSNKALPAPIEKTISKAKG
QPREPQVYTLPPSRDELTKNQVSLTCLVKGFYPSDIAVEWESNGQPENN
YKTTPPVLDSDGSFFLYSKLTVDKSRWQQGNVFSCSVMHEALHNHYTQK
SLSLSPGK;
>VPAC2 extracellular region-Fc
(SEQ ID NO: 3)
IHPECRFHLEIQEEETKCAELLRSQTEKHKACSGVWDNITCWRPANVGE
TVTVPCPKVFSNFYSKAGNISKNCTSDGWSETFPDFVDACGYSDPEDES
KITFYILVDIEGRMDEPKSSDKTHTCPPCPAPELLGGPSVFLFPPKPKD
TLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQ
VYTLPPSRDELTKNQVSLTCLVKGFYPSDIAVEWESNGQPENNYKTTPP
VLDSDGSFFLYSKLTVDKSRWQQGNVFSCSVMHEALHNHYTQKSLSLSP
GK;
Note:
The extracellular regions of PAC1, VPAC1, and VPAC2 are underlined, and the human IgG1 Fc tag is italicized.

The PAC1, VPAC1, and VPAC2 cell expression supernatant samples were centrifuged at high speed to remove impurities, and the recombinant antibody expression supernatants were purified using a Protein A column. The column was rinsed with PBS until the A280 reading dropped to the baseline. The protein of interest was eluted with 100 mM acetic acid (pH 3.5), neutralized with 1 M Tris-HCl (pH 8.0), and buffer-exchanged into a PBS solution. After confirmation by mass spectroscopy, the protein solution was aliquoted and stored for later use.

### Example 3. Hybridoma Screening for CGRP and PACAP Antibodies

In the present disclosure, hybridoma technology was used to screen for monoclonal antibodies against CGRP and monoclonal antibodies against PACAP.

The criteria for screening for CGRP antibodies of interest were: binding specifically to human CGRP, cross-reacting with rat CGRP, and inhibiting cAMP production in SK-N-MC cells.

The criteria for screening for PACAP antibodies of interest were: binding specifically to PACAP38 and PACAP27, not binding to VIP, having activity in blocking the binding of PACAP38 to its receptors PAC1, VPAC1, and VPAC2, and inhibiting cAMP production in SH-SY5Y cells.

KLH was coupled with human CGRP, rat CGRP, and PACAP38 separately to obtain immunizing agents, and TiterMax® Gold Adjuvant (Sigma Cat No. T2684) and Thermo Imject® Alum (Thermo Cat No. 77161) were used as adjuvants to cross-immunize mice. Mice with high antibody titers in serum were selected for spleen cell fusion. After fusion, the hybridoma culture supernatant was assayed based on the growth density of hybridoma cells, and antibodies that specifically bind to CGRP and PACAP were selected by screening.

The screening yielded monoclonal hybridoma cell strains C1 #, C9 #, C21 #, C28 #, P14 #, and P96 #, which exhibited good activity. Hybridoma cells in the logarithmic growth phase were collected, and their RNA was extracted using NucleoZol (MN) and reverse-transcribed (PrimeScript™ Reverse Transcriptase, Takara, cat #2680A). The resulting cDNA was PCR-amplified using mouse Ig-Primer Set (Novagen, TB326 Rev.B 0503) and then sequenced. The amino acid sequences of the CDRs and variable regions of C1 #, C9 #, C21 #, C28 #, P14 #, and P96 #are shown below.

TABLE 14
The CDRs of CGRP and PACAP antibodies

| No. | Heavy chain | | Light chain | |
|---|---|---|---|---|
| C1# | HCDR1 | TSWMN | LCDR1 | RSSQSLVHSNGNTYFH |
| | | (SEQ ID NO: 4) | | (SEQ ID NO: 7) |
| | HCDR2 | QIYPGDGYTNYNGKFRG | LCDR2 | EVSNRFS |
| | | (SEQ ID NO: 5) | | (SEQ ID NO: 8) |
| | HCDR3 | DYEAL | LCDR3 | SQSTHVPFT |
| | | (SEQ ID NO: 6) | | (SEQ ID NO: 9) |
| C9# | HCDR1 | DYYMN | LCDR1 | KASQNVGTNVA |
| | | (SEQ ID NO: 10) | | (SEQ ID NO: 13) |
| | HCDR2 | VINPYNGRTTYNRKFKG | LCDR2 | SASYRYS |
| | | (SEQ ID NO: 11) | | (SEQ ID NO: 14) |
| | HCDR3 | LLIRPHYFDY | LCDR3 | QQYNFYPYT |
| | | (SEQ ID NO: 12) | | (SEQ ID NO: 15) |
| C21# | HCDR1 | DYYMN | LCDR1 | KASQNVDTNVA |
| | | (SEQ ID NO: 16) | | (SEQ ID NO: 19) |
| | HCDR2 | DINPNNGGTNYNQKFKG | LCDR2 | SASYRYS |
| | | (SEQ ID NO: 17) | | (SEQ ID NO: 20) |
| | HCDR3 | IIIVPNYFDY | LCDR3 | QQYNNYPYT |
| | | (SEQ ID NO: 18) | | (SEQ ID NO: 21) |
| C28# | HCDR1 | DYYMN | LCDR1 | KASQNVGSNVA |
| | | (SEQ ID NO: 22) | | (SEQ ID NO: 25) |
| | HCDR2 | DINPNNGVTTYNQKFKG | LCDR2 | SASYRYS |
| | | (SEQ ID NO: 23) | | (SEQ ID NO: 26) |
| | HCDR3 | IIIVPDYFDY | LCDR3 | QQYNSYPYT |
| | | (SEQ ID NO: 24) | | (SEQ ID NO: 27) |
| P14# | HCDR1 | NYDMS | LCDR1 | KASQSVDYDGDSYMT |
| | | (SEQ ID NO: 28) | | (SEQ ID NO: 31) |
| | HCDR2 | TITGGGSYTWYPDSVKG | LCDR2 | AASNLDS |
| | | (SEQ ID NO: 29) | | (SEQ ID NO: 32) |
| | HCDR3 | LNGYDQAGAMDY | LCDR3 | QQSNEDPWT |
| | | (SEQ ID NO: 30) | | (SEQ ID NO: 33) |
| P96# | HCDR1 | DYYMH | LCDR1 | RSSSGTVTTSNYAN |
| | | (SEQ ID NO: 34) | | (SEQ ID NO: 37) |
| | HCDR2 | WIDPENGNTLYDPKFQD | LCDR2 | GTNNRAP |
| | | (SEQ ID NO: 35) | | (SEQ ID NO: 38) |
| | HCDR3 | GVMTTVEGDFDY | LCDR3 | ALWYSNHWV |
| | | (SEQ ID NO: 36) | | (SEQ ID NO: 39) |

>C1# murine heavy chain variable region
(SEQ ID NO: 40)
QVQLQQSGAELVRPGSSVKISCKASGYTFSTSWMNWVKQRPGQGLEWIGQIYPG
DGYTNYNGKFRGKATLTADKSSSTAYMQLSSLTSEDSAVYFCAGDYEALWGQGT
TLTVSS
>C1# murine light chain variable region
(SEQ ID NO: 41)
DVVMTQTPLSLPVSLGDQASISCRSSQSLVHSNGNTYFHWYLQRPGQSPKLLIYE
VSNRFSGVPDRFSGSGSGTDFTLKISRVEAEDLGVYFCSQSTHVPFTFGSGTKLEI
K
>C9# murine heavy chain variable region
(SEQ ID NO: 42)
EVQLQQSGPVLVKPGASVKMSCKASGYTFTDYYMNWVKQSLGKSLEWIGVINP
YNGRTTYNRKFKGKATLTVDKSSSTAYMELNSLTSEDSAVYYCASLLIRPHYFDY
WGQGTTLTVSS
>C9# murine light chain variable region
(SEQ ID NO: 43)
DIVMTQSQKFMATSVGDRVSVTCKASQNVGTNVAWYQQKPGQSPKALIYSASYR
YSVVPDRFTGSGSGTDFTLTIRNVQSEDLAEYHCQQYNFYPYTFGAGTKLELK
>C21# murine heavy chain variable region
(SEQ ID NO: 44)
EVQLQQSGPELVKPGASVRISCKASGYTFTDYYMNWVRQSHRKSLEWIGDINPN
NGGTNYNQKFKGKATLTVDKSSSTAYMELRSLTSEDSAVYYCASIIIVPNYFDYW
GQGTTLTVSS
>C21# murine light chain variable region
(SEQ ID NO: 45)
DIVMTQSQKFMSTSVGDRVSVTCKASQNVDTNVAWFQQRPGQSPKALIYSASYR
YSGVPDRFTGSGSGTDFTLTISNVQSEDLADYFCQQYNNYPYTFGGGTKLEIE
>C28# murine heavy chain variable region
(SEQ ID NO: 46)
EVQLQQSGPELVKPGASVKISCEASGYTFTDYYMNWVKQSHGKSLEWIGDINPN
NGVTTYNQKFKGKATLTVDKSSSTAYMELRSLTSEDSAVYYCASIIIVPDYFDYW
GQGTTLTVSS
>C28# murine light chain variable region
(SEQ ID NO: 47)
DIVMTHSQKFMSTSVGDRVSVTCKASQNVGSNVAWYQEKPGQSPKALIYSASYR
YSGVPDRFTGSGSGTDFTLTISNVQSEDLAEYFCQQYNSYPYTFGGGTKLEIK
>P14# murine heavy chain variable region
(SEQ ID NO: 48)
DVKLVESGGGLVKPGGSLKVSCAASGFAFNNYDMSWVRQTPEKRLDWVATITG
GGSYTWYPDSVKGRFTISRDNARNTLFLQMSSLRSEDTALYYCARLNGYDQAGA
MDYWGQGTSVTVSS
>P14# murine light chain variable region
(SEQ ID NO: 49)
DIVLTQSPASLAVSLGQRATISCKASQSVDYDGDSYMTWYQQKPGQPPKLLIYAA
SNLDSGIPARFSGSGSGTDFTLNIHPVEEEDAATYYCQQSNEDPWTFGGGTKLEIK
>P96# murine heavy chain variable region
(SEQ ID NO: 50)
EVQLQQSGADLVRPGALVKLSCKASGFNIKDYYMHWVKQRPEQGLEWIGWIDP
ENGNTLYDPKFQDKASITTDTSSNTAYLQLSSLTSEDTAVYYCVFGVMTTVEGDF
DYWGQGTALTVSS
>P96# murine light chain variable region
(SEQ ID NO: 51)
QAVVTQESALTTSPGETVTLTCRSSSGTVTTSNYANWVQEKPDHLFTGLIGGTNN
RAPGAPARFSGSLIGDKAALTITGAQTEDEAIYFCALWYSNHWVFGGGTKLTVL.
The variable regions of the above antibodies were fused to human
constant regions, andthe resulting chimeric antibodies were
designated C1#-CH1, C9#-CH1, C21#-CH1, C28#-CH1, P14#-CH1, and
P96#-CH1.
>Heavy chain constant region
(SEQ ID NO: 52)
ASTKGPSVFPLAPSSKSTSGGTAALGCLVKDYFPEPVTVSWNSGALTSGVHTFPAV
LQSSGLYSLSSVVTVPSSSLGTQTYICNVNHKPSNTKVDKKVEPKSCDKTHTCPP
CPAPELLGGPSVFLFPPKPKDTLYITREPEVTCVVVDVSHEDPEVKFNWYVDGVE
VHNAKTKPREEQYNSTYRVVSVLTVLHQDWLNGKEYKCKVSNKALPAPIEKTIS
KAKGQPREPQVYTLPPSRDELTKNQVSLTCLVKGFYPSDIAVEWESNGQPENNYK
TTPPVLDSDGSFFLYSKLTVDKSRWQQGNVFSCSVMHEALHNHYTQKSLSLSPG
K
>Light chain constant region
(SEQ ID NO: 53)
RTVAAPSVFIFPPSDEQLKSGTASVVCLLNNFYPREAKVQWKVDNALQSGNSQES
VTEQDSKDSTYSLSSTLTLSKADYEKHKVYACEVTHQGLSSPVTKSFNRGEC.

### Example 4. Humanization Design for Anti-Human CGRP and PACAP Monoclonal Antibodies

In the present disclosure, antibodies C1 #, C9 #, C21 #, C28 #, P14 #, and P96 #were humanized. The humanization of murine anti-human CGRP and PACAP monoclonal antibodies was performed as described in many publications in the art. In brief, based on the typical structures of the VH/VL CDRs of the murine antibodies obtained, homologous sequences of the light chain variable regions (VLs) and the heavy chain variable regions (VHs) were searched for in a human germline database and ranked in descending order according to their FR homology, and the germline with the highest FR homology was selected as the template; the CDRs of the murine antibodies were grafted onto the human template, and some amino acid residues were then mutated; the constant regions of the murine antibodies were substituted with human constant regions to obtain the final humanized molecules.

### 1. Selection of Human FRs and Mutations for C1 #

For the humanized antibodies of C1 #, the FR1, FR2, and FR3 of IGHV1-69*02 and the FR4 of IGHJ6*01 were selected as heavy chain framework region templates, and the FR1, FR2, and FR3 of IGKV2-40*01 and the FR4 of IGKJ2*01 were selected as light chain framework region templates. Optionally, the amino acid residue(s) at position(s) 1, 27, 94, 60, and/or 61 in the heavy chain variable regions of the humanized antibodies were/was substituted; and/or the amino acid residue at position 28 in the light chain variable regions of the humanized antibodies was substituted. The positions were determined according to the Kabat numbering scheme. See the table below.

TABLE 15
Mutations for humanized antibodies of C1#

| | |
|---|---|
| C1#.VL | C1#.VH |

| | | | |
|---|---|---|---|
| L1 | | H1 | Q1E, G27Y, R94G + N60S |
| L2 | N28S | H2 | Q1E, G27Y, R94G + N60Q |
| | | H3 | Q1E, G27Y, R94G + G61V |

Illustratively, R94G means mutating the R at position 94 back to G according to the Kabat numbering scheme; the mutation(s) at position N60 or G61 is/are optimization of the CDRs. This applies hereinafter.

The specific sequences of the variable regions of the antibodies obtained by humanizing murine antibody C1 #are shown below.

>C1H1
(SEQ ID NO: 54)
EVQLVQSGAEVKKPGSSVKVSCKASGYTFSTSWMNWVRQAPGQGLEWMG
QIYPGDGYTNYSGKFRGRVTITADKSTSTAYMELSSLRSEDTAVYYCAG
DYEALWGQGTTVTVSS
>C1H2
(SEQ ID NO: 55)
EVQLVQSGAEVKKPGSSVKVSCKASGYTFSTSWMNWVRQAPGQGLEWMG
QIYPGDGYTNYQGKFRGRVTITADKSTSTAYMELSSLRSEDTAVYYCAG
DYEALWGQGTTVTVSS
>C1H3
(SEQ ID NO: 56)
EVQLVQSGAEVKKPGSSVKVSCKASGYTFSTSWMNWVRQAPGQGLEWMG
QIYPGDGYTNYNVKFRGRVTITADKSTSTAYMELSSLRSEDTAVYYCAG
DYEALWGQGTTVTVSS
>C1L1
(SEQ ID NO: 57)
DIVMTQTPLSLPVTPGEPASISCRSSQSLVHSNGNTYFHWYLQKPGQSP
QLLIYEVSNRFSGVPDRFSGSGSGTDFTLKISRVEAEDVGVYYCSQSTH
VPFTFGQGTKLEIK
>C1L2
(SEQ ID NO: 58)
DIVMTQTPLSLPVTPGEPASISCRSSQSLVHSSGNTYFHWYLQKPGQSP
QLLIYEVSNRFSGVPDRFSGSGSGTDFTLKISRVEAEDVGVYYCSQSTH
VPFTFGQGTKLEIK.

### 2. Selection of Human FRs and Mutations for C9 #

For the humanized antibodies of C9 #, the FR1, FR2, and FR3 of IGHV1-3*01 and the FR4 of IGHJ6*01 were selected as heavy chain framework region templates, and the FR1, FR2, and FR3 of IGKV1-27*01 and the FR4 of IGKJ2*01 were selected as light chain framework region templates. Optionally, the amino acid residue(s) at position(s) 1, 48, 67, 69, 71, 73, 94, 54, and/or 55 in the heavy chain variable regions of the humanized antibodies were/was substituted; and/or the amino acid residue(s) at position(s) 43, 46, and/or 87 in the light chain variable regions of the humanized antibodies were/was substituted. The positions were determined according to the Kabat numbering scheme. See the table below.

TABLE 16
Mutations for humanized antibodies of C9#

| C9#.VL | | C9#.VH | |
|---|---|---|---|
| L1 | V43S, L46A, Y87H | H1 | Q1E, R71V, T73K, R94S + G55V |
| | | H2 | Q1E, M48I, V67A, I69L, R71V, T73K, R94S + G55V |
| | | H3 | Q1E, R71V, T73K, R94S + N54Q |
| | | H4 | Q1E, M48I, V67A, I69L, R71V, T73K, R94S + N54Q |
| | | H5 | Q1E, R71V, T73K, R94S + N54S |
| | | H6 | Q1E, M48I, V67A, I69L, R71V, T73K, R94S + N54S |
| | | H7 | Q1E, R71V, T73K, R94S + N54T |
| | | H8 | Q1E, M48I, V67A, I69L, R71V, T73K, R94S + N54T |

The specific sequences of the variable regions of the antibodies obtained by humanizing murine antibody C9 #are shown below.

>C9H1
(SEQ ID NO: 59)
EVQLVQSGAEVKKPGASVKVSCKASGYTFTDYYMNWVRQAPGQRLEWMGVIN
PYNVRTTYNRKFKGRVTITVDKSASTAYMELSSLRSEDTAVYYCASLLIRPHYFDY
WGQGTTVTVSS
>C9H2
(SEQ ID NO: 60)
EVQLVQSGAEVKKPGASVKVSCKASGYTFTDYYMNWVRQAPGQRLEWIGVINP
YNVRTTYNRKFKGRATLTVDKSASTAYMELSSLRSEDTAVYYCASLLIRPHYFDY
WGQGTTVTVSS
>C9H3
(SEQ ID NO: 61)
EVQLVQSGAEVKKPGASVKVSCKASGYTFTDYYMNWVRQAPGQRLEWMGVIN
PYQGRTTYNRKFKGRVTITVDKSASTAYMELSSLRSEDTAVYYCASLLIRPHYFDY
WGQGTTVTVSS
>C9H4
(SEQ ID NO: 62)
EVQLVQSGAEVKKPGASVKVSCKASGYTFTDYYMNWVRQAPGQRLEWIGVINP
YQGRTTYNRKFKGRATLTVDKSASTAYMELSSLRSEDTAVYYCASLLIRPHYFDY
WGQGTTVTVSS
>C9H5
(SEQ ID NO: 63)
EVQLVQSGAEVKKPGASVKVSCKASGYTFTDYYMNWVRQAPGQRLEWMGVIN
PYSGRTTYNRKFKGRVTITVDKSASTAYMELSSLRSEDTAVYYCASLLIRPHYFDY
WGQGTTVTVSS
>C9H6
(SEQ ID NO: 64)
EVQLVQSGAEVKKPGASVKVSCKASGYTFTDYYMNWVRQAPGQRLEWIGVINP
YSGRTTYNRKFKGRATLTVDKSASTAYMELSSLRSEDTAVYYCASLLIRPHYFDY
WGQGTTVTVSS
>C9H7
(SEQ ID NO: 65)
EVQLVQSGAEVKKPGASVKVSCKASGYTFTDYYMNWVRQAPGQRLEWMGVIN
PYTGRTTYNRKFKGRVTITVDKSASTAYMELSSLRSEDTAVYYCASLLIRPHYFDY
WGQGTTVTVSS
>C9H8
(SEQ ID NO: 66)
EVQLVQSGAEVKKPGASVKVSCKASGYTFTDYYMNWVRQAPGQRLEWIGVINP
YTGRTTYNRKFKGRATLTVDKSASTAYMELSSLRSEDTAVYYCASLLIRPHYFDY
WGQGTTVTVSS
>C9L1
(SEQ ID NO: 67)
DIQMTQSPSSLSASVGDRVTITCKASQNVGTNVAWYQQKPGKSPKALIYSASYRY
SGVPSRFSGSGSGTDFTLTISSLQPEDVATYHCQQYNFYPYTFGQGTKLEIK.

### 3. Selection of Human FRs and Mutations for C21 #

For the humanized antibodies of C21 #, the FR1, FR2, and FR3 of IGHV1-3*01 and the FR4 of IGHJ6*01 were selected as heavy chain framework region templates, and the FR1, FR2, and FR3 of IGKV1-16*01 and the FR4 of IGKJ4*01 were selected as light chain framework region templates. Optionally, the amino acid residue(s) at position(s) 1, 44, 48, 67, 69, 71, 73, 94, 54, and/or 55 in the heavy chain variable regions of the humanized antibodies were/was substituted; and/or the amino acid residue(s) at position(s) 43, 46, and/or 100 in the light chain variable regions of the humanized antibodies were/was substituted. The positions were determined according to the Kabat numbering scheme. See the table below.

TABLE 17
Mutations for humanized antibodies of C21#

| C21#.VL | | C21#.VH | |
|---|---|---|---|
| L1 | | H1 | Q1E, M48I, V67A, I69L, R71V, T73K, R94S + G55V |
| L2 | A43S, S46A | H2 | Q1E, M48I, V67A, I69L, R71V, T73K, R94S + N54Q |
| L3 | G100C | H3 | Q1E, R71V, T73K, R94S + N54S |
| | | H4 | Q1E, M48I, V67A, I69L, R71V, T73K, R94S + N54T |
| | | H5 | Q1E, R44C, M48I, V67A, I69L, R71V, T73K, R94S + N54Q |

The specific sequences of the variable regions of the antibodies obtained by humanizing murine antibody C21 #are shown below.

>C21H1
(SEQ ID NO: 68)
EVQLVQSGAEVKKPGASVKVSCKASGYTFTDYYMNWVRQAPGQRLEWIGDINP
NNVGTNYNQKFKGRATLTVDKSASTAYMELSSLRSEDTAVYYCASIIIVPNYFDY
WGQGTTVTVSS
>C21H2
(SEQ ID NO: 69)
EVQLVQSGAEVKKPGASVKVSCKASGYTFTDYYMNWVRQAPGQRLEWIGDINP
NQGGTNYNQKFKGRATLTVDKSASTAYMELSSLRSEDTAVYYCASIIIVPNYFDY
WGQGTTVTVSS
>C21H3
(SEQ ID NO: 70)
EVQLVQSGAEVKKPGASVKVSCKASGYTFTDYYMNWVRQAPGQRLEWMGDIN
PNSGGTNYNQKFKGRVTITVDKSASTAYMELSSLRSEDTAVYYCASIIIVPNYFDY
WGQGTTVTVSS
>C21H4
(SEQ ID NO: 71)
EVQLVQSGAEVKKPGASVKVSCKASGYTFTDYYMNWVRQAPGQRLEWIGDINP
NTGGTNYNQKFKGRATLTVDKSASTAYMELSSLRSEDTAVYYCASIIIVPNYFDY
WGQGTTVTVSS
>C21H5
(SEQ ID NO: 72)
EVQLVQSGAEVKKPGASVKVSCKASGYTFTDYYMNWVRQAPGQCLEWIGDINP
NQGGTNYNQKFKGRATLTVDKSASTAYMELSSLRSEDTAVYYCASIIIVPNYFDY
WGQGTTVTVSS
>C21L1
(SEQ ID NO: 73)
DIQMTQSPSSLSASVGDRVTITCKASQNVDTNVAWFQQKPGKAPKSLIYSASYRY
SGVPSRFSGSGSGTDFTLTISSLQPEDFATYYCQQYNNYPYTFGGGTKVEIK
>C21L2
(SEQ ID NO: 74)
DIQMTQSPSSLSASVGDRVTITCKASQNVDTNVAWFQQKPGKSPKALIYSASYRY
SGVPSRFSGSGSGTDFTLTISSLQPEDFATYYCQQYNNYPYTFGGGTKVEIK
>C21L3
(SEQ ID NO: 75)
DIQMTQSPSSLSASVGDRVTITCKASQNVDTNVAWFQQKPGKAPKSLIYSASYRY
SGVPSRFSGSGSGTDFTLTISSLQPEDFATYYCQQYNNYPYTFGCGTKVEIK.

### 4. Selection of Human FRs and Mutations for C28 #

For the humanized antibodies of C28 #, the FR1, FR2, and FR3 of IGHV1-3*01 and the FR4 of IGHJ6*01 were selected as heavy chain framework region templates, and the FR1, FR2, and FR3 of IGKV1-12*01 and the FR4 of IGKJ4*01 were selected as light chain framework region templates. Optionally, the amino acid residue(s) at position(s) 1, 44, 48, 67, 69, 71, 73, 94, 54, and/or 55 in the heavy chain variable regions of the humanized antibodies were/was substituted; and/or the amino acid residue(s) at position(s) 43, 46, and/or 100 in the light chain variable regions of the humanized antibodies were/was substituted. The positions were determined according to the Kabat numbering scheme. See the table below.

TABLE 18
Mutations for humanized antibodies of C28#

| C28#.VL | | C28#.VH | |
|---|---|---|---|
| L1 | A43S, S46A | H1 | Q1E, R71V, T73K, R94S + G55V |
| L2 | A43S, S46A, G100C | H2 | Q1E, M48I, V67A, I69L, R71V, T73K, R94S + G55V |
| | | H3 | Q1E, R71V, T73K, R94S + N54Q |
| | | H4 | Q1E, M48I, V67A, I69L, R71V, T73K, R94S + N54Q |
| | | H5 | Q1E, M48I, V67A, I69L, R71V, T73K, R94S + N54S |
| | | H6 | Q1E, R71V, T73K, R94S + N54T |
| | | H7 | Q1E, M48I, V67A, I69L, R71V, T73K, R94S + N54T |
| | | H8 | Q1E, R44C, R71V, T73K, R94S + N54T |

The specific sequences of the variable regions of the antibodies obtained by humanizing murine antibody C28 #are shown below.

>C28H1
(SEQ ID NO: 76)
EVQLVQSGAEVKKPGASVKVSCKASGYTFTDYYMNWVRQAPGQRLEWMGDIN
PNNVVTTYNQKFKGRVTITVDKSASTAYMELSSLRSEDTAVYYCASIIIVPDYFDY
WGQGTTVTVSS
>C28H2
(SEQ ID NO: 77)
EVQLVQSGAEVKKPGASVKVSCKASGYTFTDYYMNWVRQAPGQRLEWIGDINP
NNVVTTYNQKFKGRATLTVDKSASTAYMELSSLRSEDTAVYYCASIIIVPDYFDY
WGQGTTVTVSS
>C28H3
(SEQ ID NO: 78)
EVQLVQSGAEVKKPGASVKVSCKASGYTFTDYYMNWVRQAPGQRLEWMGDIN
PNQGVTTYNQKFKGRVTITVDKSASTAYMELSSLRSEDTAVYYCASIIIVPDYFDY
WGQGTTVTVSS
>C28H4
(SEQ ID NO: 79)
EVQLVQSGAEVKKPGASVKVSCKASGYTFTDYYMNWVRQAPGQRLEWIGDINP
NQGVTTYNQKFKGRATLTVDKSASTAYMELSSLRSEDTAVYYCASIIIVPDYFDY
WGQGTTVTVSS
>C28H5
(SEQ ID NO: 80)
EVQLVQSGAEVKKPGASVKVSCKASGYTFTDYYMNWVRQAPGQRLEWIGDINP
NSGVTTYNQKFKGRATLTVDKSASTAYMELSSLRSEDTAVYYCASIIIVPDYFDY
WGQGTTVTVSS
>C28H6
(SEQ ID NO: 81)
EVQLVQSGAEVKKPGASVKVSCKASGYTFTDYYMNWVRQAPGQRLEWMGDIN
PNTGVTTYNQKFKGRVTITVDKSASTAYMELSSLRSEDTAVYYCASIIIVPDYFDY
WGQGTTVTVSS
>C28H7
(SEQ ID NO: 82)
EVQLVQSGAEVKKPGASVKVSCKASGYTFTDYYMNWVRQAPGQRLEWIGDINP
NTGVTTYNQKFKGRATLTVDKSASTAYMELSSLRSEDTAVYYCASIIIVPDYFDY
WGQGTTVTVSS
>C28H8
(SEQ ID NO: 83)
EVQLVQSGAEVKKPGASVKVSCKASGYTFTDYYMNWVRQAPGQCLEWMGDIN
PNTGVTTYNQKFKGRVTITVDKSASTAYMELSSLRSEDTAVYYCASIIIVPDYFDY
WGQGTTVTVSS
>C28L1
(SEQ ID NO: 84)
DIQMTQSPSSVSASVGDRVTITCKASQNVGSNVAWYQQKPGKSPKALIYSASYRY
SGVPSRFSGSGSGTDFTLTISSLQPEDFATYYCQQYNSYPYTFGGGTKVEIK
>C28L2
(SEQ ID NO: 85)
DIQMTQSPSSVSASVGDRVTITCKASQNVGSNVAWYQQKPGKSPKALIYSASYRY
SGVPSRFSGSGSGTDFTLTISSLQPEDFATYYCQQYNSYPYTFGCGTKVEIK.

### 5. Selection of Human FR5 and Mutations for P14 #

For the humanized antibodies of P14 #, the FR1, FR2, and FR3 of IGHV3-21*01 and the FR4 of IGHJ6*01 were selected as heavy chain framework region templates, and the FR1, FR2, and FR3 of IGKV4-1*01 and the FR4 of IGKJ4*01 were selected as light chain framework region templates. Optionally, the amino acid residue(s) at position(s) 13, 28, 30, 49, and/or 96 in the heavy chain variable regions of the humanized antibodies were/was substituted; and/or the amino acid residue(s) at position(s) 4, 58, and/or 53 in the light chain variable regions of the humanized antibodies were/was substituted. The positions were determined according to the Kabat numbering scheme. See the table below.

TABLE 19
Mutations for humanized antibodies of P14#

| P14#.VL | | P14#.VH | |
|---|---|---|---|
| L1 | | H1 | T28A, S30N, S49A + N96T |
| L2 | M4L,V58I | H2 | K13Q, T28A, S30N, S49A + N96T |
| L3 | M4L, V58I + N53H | | |

The specific sequences of the variable regions of the antibodies obtained by humanizing murine antibody P14 #are shown below.

>P14H1
(SEQ ID NO: 86)
EVQLVESGGGLVKPGGSLRLSCAASGFAFNNYDMSWVRQAPGKGLEWVATITGG
GSYTWYPDSVKGRFTISRDNAKNSLYLQMNSLRAEDTAVYYCARLTGYDQAGA
MDYWGQGTTVTVSS;
>P14H2
(SEQ ID NO: 87)
EVQLVESGGGLVQPGGSLRLSCAASGFAFNNYDMSWVRQAPGKGLEWVATITGG
GSYTWYPDSVKGRFTISRDNAKNSLYLQMNSLRAEDTAVYYCARLTGYDQAGA
MDYWGQGTTVTVSS;
>P14L1
(SEQ ID NO: 88)
DIVMTQSPDSLAVSLGERATINCKASQSVDYDGDSYMTWYQQKPGQPPKLLIYA
ASNLDSGVPDRFSGSGSGTDFTLTISSLQAEDVAVYYCQQSNEDPWTFGGGTKVE
IK;
>P14L2
(SEQ ID NO: 89)
DIVLTQSPDSLAVSLGERATINCKASQSVDYDGDSYMTWYQQKPGQPPKLLIYAA
SNLDSGIPDRFSGSGSGTDFTLTISSLQAEDVAVYYCQQSNEDPWTFGGGTKVEIK;
>P14L3
(SEQ ID NO: 90)
DIVLTQSPDSLAVSLGERATINCKASQSVDYDGDSYMTWYQQKPGQPPKLLIYAA
SHLDSGIPDRFSGSGSGTDFTLTISSLQAEDVAVYYCQQSNEDPWTFGGGTKVEIK.

### 6. Selection of Human FRs and Mutations for P96 #

For the humanized antibodies of P96 #, the FR1, FR2, and FR3 of IGHV1-69-2*01 and the FR4 of IGHJ6*01 were selected as heavy chain framework region templates, and the FR1, FR2, and FR3 of IGLV7-43*01 and the FR4 of IGLJ2*01 were selected as light chain framework region templates. Optionally, the amino acid residue(s) at position(s) 24, 27, 28, 29, 30, 71, 76, 93, 94, and/or 55 in the heavy chain variable regions of the humanized antibodies were/was substituted; and/or the amino acid residue(s) at position(s) 36, 44, 46, 49, 57, and/or 58 in the light chain variable regions of the humanized antibodies were/was substituted. The positions were determined according to the Kabat numbering scheme. See the table below.

TABLE 20
Mutations for humanized antibodies of P96#

| P96#.VL | | P96#.VH | |
|---|---|---|---|
| L1 | F36V, P44F, A46G, Y49G, | H1 | V24A, Y27F, T28N, F29I, T30K, A71T, D76N, A93V, |
| | W57G, T58A | | T94F + G55D |

The specific sequences of the variable regions of the antibodies obtained by humanizing murine antibody P96 #are shown below.

>P96H1
(SEQ ID NO: 91)
EVQLVQSGAEVKKPGATVKISCKASGFNIKDYYMHWVQQAPGKGLEWM
GWIDPENDNTLYDPKFQDRVTITTDTSTNTAYMELSSLRSEDTAVYYC
VFGVMTTVEGDFDYWGQGTTVTVSS;
>P96L1
(SEQ ID NO: 92)
ETVVTQEPSLTVSPGGTVTLTCRSSSGTVTTSNYANWVQQKPGQAFRG
LIGGTNNRAPGAPARFSGSLLGGKAALTLSGVQPEDEAEYYCALWYSN
HWVFGGGTKLTVL

The amino acid sequences of substituted CDRs in the humanized antibodies are shown in the table below.

TABLE 21
The amino acid sequences of substituted CDRs

| CDR | Amino acid sequence | CDR | Amino acid sequence |
|---|---|---|---|
| C1H1 CDR2 | QIYPGDGYTNYSGKFRG | C21H1 CDR2 | DINPNNVGTNYNQKFKG |
| | (SEQ ID NO: 93) | | (SEQ ID NO: 100) |
| C1H2 CDR2 | QIYPGDGYTNYQGKFRG | C21H2 CDR2 | DINPNQGGTNYNQKFKG |
| | (SEQ ID NO: 94) | | (SEQ ID NO: 101) |
| C1H3 CDR2 | QIYPGDGYTNYNVKFRG | C21H3 CDR2 | DINPNSGGTNYNQKFKG |
| | (SEQ ID NO: 95) | | (SEQ ID NO: 102) |
| C9H1 CDR2 | VINPYNVRTTYNRKFKG | C21H4 CDR2 | DINPNTGGTNYNQKFKG |
| | (SEQ ID NO: 96) | | (SEQ ID NO: 192) |
| C9H2 CDR2 | (SEQ ID NO: 96) | C21H5 CDR2 | (SEQ ID NO: 101) |
| C9H3 CDR2 | VINPYQGRTTYNRKFKG | C28H1 CDR2 | DINPNNVVTTYNQKFKG |
| | (SEQ ID NO: 97) | | (SEQ ID NO: 103) |
| C9H4 CDR2 | (SEQ ID NO: 97) | C28H2 CDR2 | (SEQ ID NO: 103) |
| C9H5 CDR2 | VINPYSGRTTYNRKFKG | C28H3 CDR2 | DINPNQGVTTYNQKFKG |
| | (SEQ ID NO: 98) | | (SEQ ID NO: 104) |
| C9H6 CDR2 | (SEQ ID NO: 98) | C28H4 CDR2 | (SEQ ID NO: 104) |
| C9H7 CDR2 | VINPYTGRTTYNRKFKG | C28H5 CDR2 | DINPNSGVTTYNQKFKG |
| | (SEQ ID NO: 99) | | (SEQ ID NO: 105) |
| C9H8 CDR2 | (SEQ ID NO: 99) | C28H6 CDR2 | DINPNTGVTTYNQKFKG |
| | | | (SEQ ID NO: 106) |
| P14H1 CDR3 | LTGYDQAGAMDY | C28H7 CDR2 | (SEQ ID NO: 106) |
| | (SEQ ID NO: 107) | | |
| P14H2 CDR3 | (SEQ ID NO: 107) | C28H8 CDR2 | (SEQ ID NO: 106) |
| P14L3 CDR2 | AASHLDS | P96H1 CDR2 | WIDPENDNTLYDPKFQD |
| | (SEQ ID NO: 108) | | (SEQ ID NO: 109) |

After the heavy chain variable regions and the light chain variable regions in the above groups were humanized, they were arbitrarily paired and fused to human constant regions to form humanized antibodies. The amino acid sequence of the heavy chain constant region of the humanized antibodies is set forth in SEQ ID NO: 52, and the amino acid sequence of the light chain constant region is set forth in SEQ ID NO: 53. C1H1L1 represents a humanized antibody using the H1 (C1H1) and L1 (C1L1) of C1. This applies to the others.

### Example 5. Preparation of Anti-CGRP-PACAP Bispecific Antibodies

C21H5L3, C28H8L2, and C28H6L1 were each combined with the variable regions of P14H2L3 and IgG₁ (YTE) (M252Y/S254T/T256E), an IgG₁ mutant, to form three bispecific antibodies: CP-1, CP-2, and CP-3, respectively. C28H8L2 was combined with the variable regions of P14H2L3 and IgG₁ to form a bispecific antibody: CP-4.

CP-1 was a symmetrically structured molecule comprising two identical heavy chains and two identical light chains:
heavy chain: [VH (P14H2)]-[IgG₁ (CH1)]-[VH (C21H5)]-[linker 1]-[VL (C21L3)]-[linker 2]-[IgG₁ (YTE) Fc]; and
light chain: [VL (P14L3)]-[CL], and it is schematically shown in FIG. 1A.

CP-2 was a symmetrically structured molecule comprising two identical heavy chains and two identical light chains:
heavy chain: [VH (P14H2)]-[IgG₁ (CH1)]-[VH (C28H8)]-[linker 1]-[VL (C28L2)]-[linker 2]-[IgG₁ (YTE) Fc]; and
light chain: [VL (P14L3)]-[CL], and it is schematically shown in FIG. 1A.

CP-4 was a symmetrically structured molecule comprising two identical heavy chains and two identical light chains:
heavy chain: [VH (P14H2)]-[IgG₁ (CH1)]-[VH (C28H8)]-[linker 1]-[VL (C28L2)]-[linker 2]-[IgG₁ Fc]; and
light chain: [VL (P14L3)]-[CL], and it is schematically shown in FIG. 1A.

>IgG₁ (YTE) Fc
(SEQ ID NO: 110)
DKTHTCPPCPAPELLGGPSVFLFPPKPKDTLYITREPEVTCVVVDVSHEDPEVKFN
WYVDGVEVHNAKTKPREEQYNSTYRVVSVLTVLHQDWLNGKEYKCKVSNKAL
PAPIEKTISKAKGQPREPQVYTLPPSRDELTKNQVSLTCLVKGFYPSDIAVEWESNG
QPENNYKTTPPVLDSDGSFFLYSKLTVDKSRWQQGNVFSCSVMHEALHNHYTQK
SLSLSPGK;
>Linker 1:
(SEQ ID NO: 189)
GGGGSGGGGSGGGGS;
>Linker 2:
(SEQ ID NO: 186)
GGG;
>CP-1 heavy chain
(SEQ ID NO: 111)
EVqLVESGGGLVQPGGSLRLSCAASGFAFNNYDMSWVRQAPGKGLEWVATITGG
GSYTWYPDSVKGRFTISRDNAKNSLYLQMNSLRAEDTAVYYCARLTGYDQAGA
MDYWGQGTTVTVSSASTKGPSVFPLAPSSKSTSGGTAALGCLVKDYFPEPVTVS
WNSGALTSGVHTFPAVLQSSGLYSLSSVVTVPSSSLGTQTYICNVNHKPSNTKVD
KKVEPKSCEVQLVQSGAEVKKPGASVKVSCKASGYTFTDYYMNWVRQAPGQC
LEWIGDINPNQGGTNYNQKFKGRATLTVDKSASTAYMELSSLRSEDTAVYYCASII
IVPNYFDYWGQGTTVTVSSGGGGSGGGGSGGGGSDIQMTQSPSSLSASVGDRVTI
TCKASQNVDTNVAWFQQKPGKAPKSLIYSASYRYSGVPSRFSGSGSGTDFTLTISS
LQPEDFATYYCQQYNNYPYTFGCGTKVEIKGGGDKTHTCPPCPAPELLGGPSVFL
FPPKPKDTLYITREPEVTCVVVDVSHEDPEVKFNWYVDGVEVHNAKTKPREEQY
NSTYRVVSVLTVLHQDWLNGKEYKCKVSNKALPAPIEKTISKAKGQPREPQVYT
LPPSRDELTKNQVSLTCLVKGFYPSDIAVEWESNGQPENNYKTTPPVLDSDGSFFL
YSKLTVDKSRWQQGNVFSCSVMHEALHNHYTQKSLSLSPGK;
>CP-1 light chain
(SEQ ID NO: 112)
DIVLTQSPDSLAVSLGERATINCKASQSVDYDGDSYMTWYQQKPGQPPKLLIYAA
SHLDSGIPDRFSGSGSGTDFTLTISSLQAEDVAVYYCQQSNEDPWTFGGGTKVEIK
RTVAAPSVFIFPPSDEQLKSGTASVVCLLNNFYPREAKVQWKVDNALQSGNSQES
VTEQDSKDSTYSLSSTLTLSKADYEKHKVYACEVTHQGLSSPVTKSFNRGEC;
>CP-2 heavy chain
(SEQ ID NO: 113)
EVQLVESGGGLVQPGGSLRLSCAASGFAFNNYDMSWVRQAPGKGLEWVATITGG
GSYTWYPDSVKGRFTISRDNAKNSLYLQMNSLRAEDTAVYYCARLTGYDQAGA
MDYWGQGTTVTVSSASTKGPSVFPLAPSSKSTSGGTAALGCLVKDYFPEPVTVS
WNSGALTSGVHTFPAVLQSSGLYSLSSVVTVPSSSLGTQTYICNVNHKPSNTKVD
KKVEPKSCEVQLVQSGAEVKKPGASVKVSCKASGYTFTDYYMNWVRQAPGQC
LEWMGDINPNTGVTTYNQKFKGRVTITVDKSASTAYMELSSLRSEDTAVYYCASI
IIVPDYFDYWGQGTTVTVSSGGGGSGGGGSGGGGSDIQMTQSPSSVSASVGDRV
TITCKASQNVGSNVAWYQQKPGKSPKALIYSASYRYSGVPSRFSGSGSGTDFTLTI
SSLQPEDFATYYCQQYNSYPYTFGCGTKVEIKGGGDKTHTCPPCPAPELLGGPSV
FLFPPKPKDTLYITREPEVTCVVVDVSHEDPEVKFNWYVDGVEVHNAKTKPREE
QYNSTYRVVSVLTVLHQDWLNGKEYKCKVSNKALPAPIEKTISKAKGQPREPQV
YTLPPSRDELTKNQVSLTCLVKGFYPSDIAVEWESNGQPENNYKTTPPVLDSDGSF
FLYSKLTVDKSRWQQGNVFSCSVMHEALHNHYTQKSLSLSPGK;
>CP-2 light chain
(SEQ ID NO: 114)
DIVLTQSPDSLAVSLGERATINCKASQSVDYDGDSYMTWYQQKPGQPPKLLIYAA
SHLDSGIPDRFSGSGSGTDFTLTISSLQAEDVAVYYCQQSNEDPWTFGGGTKVEIK
RTVAAPSVFIFPPSDEQLKSGTASVVCLLNNFYPREAKVQWKVDNALQSGNSQES
VTEQDSKDSTYSLSSTLTLSKADYEKHKVYACEVTHQGLSSPVTKSFNRGEC;
>IgG₁ Fc
(SEQ ID NO: 193)
DKTHTCPPCPAPELLGGPSVFLFPPKPKDTLMISRTPEVTCVVVDVSHEDPEVKFN
WYVDGVEVHNAKTKPREEQYNSTYRVVSVLTVLHQDWLNGKEYKCKVSNKAL
PAPIEKTISKAKGQPREPQVYTLPPSREEMTKNQVSLTCLVKGFYPSDIAVEWESN
GQPENNYKTTPPVLDSDGSFFLYSKLTVDKSRWQQGNVFSCSVMHEALHNHYTQ
KSLSLSPGK;
>CP-4 heavy chain
(SEQ ID NO: 194)
EVQLVESGGGLVQPGGSLRLSCAASGFAFNNYDMSWVRQAPGKGLEWVATITGG
GSYTWYPDSVKGRFTISRDNAKNSLYLQMNSLRAEDTAVYYCARLTGYDQAGA
MDYWGQGTTVTVSSASTKGPSVFPLAPSSKSTSGGTAALGCLVKDYFPEPVTVS
WNSGALTSGVHTFPAVLQSSGLYSLSSVVTVPSSSLGTQTYICNVNHKPSNTKVD
KKVEPKSCEVQLVQSGAEVKKPGASVKVSCKASGYTFTDYYMNWVRQAPGQC
LEWMGDINPNTGVTTYNQKFKGRVTITVDKSASTAYMELSSLRSEDTAVYYCASI
IIVPDYFDYWGQGTTVTVSSGGGGSGGGGSGGGGSDIQMTQSPSSVSASVGDRV
TITCKASQNVGSNVAWYQQKPGKSPKALIYSASYRYSGVPSRFSGSGSGTDFTLTI
SSLQPEDFATYYCQQYNSYPYTFGCGTKVEIKGGGDKTHTCPPCPAPELLGGPSV
FLFPPKPKDTLMISRTPEVTCVVVDVSHEDPEVKFNWYVDGVEVHNAKTKPREE
QYNSTYRVVSVLTVLHQDWLNGKEYKCKVSNKALPAPIEKTISKAKGQPREPQV
YTLPPSREEMTKNQVSLTCLVKGFYPSDIAVEWESNGQPENNYKTTPPVLDSDGS
FFLYSKLTVDKSRWQQGNVFSCSVMHEALHNHYTQKSLSLSPGK;
>CP-4 light chain.
(SEQ ID NO: 114)

CP-3 was an asymmetrically structured molecule, and the intact molecule had a total of four chains:
chain 1: [VH (P14H2)]-[IgG₁ (CH1)]-[IgG₁Fc (S354C, T366W, YTE)];
chain 2: [VL (P14L3)]-[CL];
chain 3: [VH (C28H6)]-[linker 3]-[Titin chain]-[IgG₁Fc (Y349C, T366S, L368A, Y407V, YTE)]; and
chain 4: [VL (C28L1)]-[linker 4]-[Obscurin chain], and it is schematically shown in FIG. 1B, where Obscurin is abbreviated as Ob.

In the molecule:

>Titin chain:
(T.16, SEQ ID NO: 137)
>Ob chain:
(O28, SEQ ID NO: 175)
>IgG₁ Fc (S354C, T366W, and YTE)
(SEQ ID NO: 115)
DKTHTCPPCPAPELLGGPSVFLFPPKPKDTLYITREPEVTCVVVDVSHEDPEVKFN
WYVDGVEVHNAKTKPREEQYNSTYRVVSVLTVLHQDWLNGKEYKCKVSNKAL
PAPIEKTISKAKGQPREPQVYTLPPCRDELTKNQVSLWCLVKGFYPSDIAVEWESN
GQPENNYKTTPPVLDSDGSFFLYSKLTVDKSRWQQGNVFSCSVMHEALHNHYTQ
KSLSLSPGK;
>IgG₁ Fc (Y349C, T366S, L368A, Y407V, and YTE)
(SEQ ID NO: 116)
DKTHTCPPCPAPELLGGPSVFLFPPKPKDTLYITREPEVTCVVVDVSHEDPEVKFN
WYVDGVEVHNAKTKPREEQYNSTYRVVSVLTVLHQDWLNGKEYKCKVSNKAL
PAPIEKTISKAKGQPREPQVCTLPPSRDELTKNQVSLSCAVKGFYPSDIAVEWESN
GQPENNYKTTPPVLDSDGSFFLVSKLTVDKSRWQQGNVFSCSVMHEALHNHYTQ
KSLSLSPGK;
>Linker 3 and linker 4:
(SEQ ID NO: 187)
GGGGS;
>CP-3 chain 1
(SEQ ID NO: 117)
EVQLVESGGGLVQPGGSLRLSCAASGFAFNNYDMSWVRQAPGKGLEWVATITGG
GSYTWYPDSVKGRFTISRDNAKNSLYLQMNSLRAEDTAVYYCARLTGYDQAGA
MDYWGQGTTVTVSSASTKGPSVFPLAPSSKSTSGGTAALGCLVKDYFPEPVTVS
WNSGALTSGVHTFPAVLQSSGLYSLSSVVTVPSSSLGTQTYICNVNHKPSNTKVD
KKVEPKSCDKTHTCPPCPAPELLGGPSVFLFPPKPKDTLYITREPEVTCVVVDVSH
EDPEVKFNWYVDGVEVHNAKTKPREEQYNSTYRVVSVLTVLHQDWLNGKEYK
CKVSNKALPAPIEKTISKAKGQPREPQVYTLPPCRDELTKNQVSLWCLVKGFYPS
DIAVEWESNGQPENNYKTTPPVLDSDGSFFLYSKLTVDKSRWQQGNVFSCSVMH
EALHNHYTQKSLSLSPGK;
>CP-3 chain 2
(SEQ ID NO: 118)
DIVLTQSPDSLAVSLGERATINCKASQSVDYDGDSYMTWYQQKPGQPPKLLIYAA
SHLDSGIPDRFSGSGSGTDFTLTISSLQAEDVAVYYCQQSNEDPWTFGGGTKVEIK
RTVAAPSVFIFPPSDEQLKSGTASVVCLLNNFYPREAKVQWKVDNALQSGNSQES
VTEQDSKDSTYSLSSTLTLSKADYEKHKVYACEVTHQGLSSPVTKSFNRGEC;
>CP-3 chain 3
(SEQ ID NO: 119)
EVQLVQSGAEVKKPGASVKVSCKASGYTFTDYYMNWVRQAPGQRLEWMGDIN
PNTGVTTYNQKFKGRVTITVDKSASTAYMELSSLRSEDTAVYYCASIIIVPDYFDY
WGQGTTVTVSSGGGGSGIPPKIECLPIDISIDEGKVLTVASAFTGEPTPEVTWSTGG
RKIHSQEQGRFHIENTDDSTTLTIKDVQKQDGGLYTLTLRNEFGSDSATVNIHIRSI
DKTHTCPPCPAPELLGGPSVFLFPPKPKDTLYITREPEVTCVVVDVSHEDPEVKEN
WYVDGVEVHNAKTKPREEQYNSTYRVVSVLTVLHQDWLNGKEYKCKVSNKAL
PAPIEKTISKAKGQPREPQVCTLPPSRDELTKNQVSLSCAVKGFYPSDIAVEWESN
GQPENNYKTTPPVLDSDGSFFLVSKLTVDKSRWQQGNVFSCSVMHEALHNHYTQ
KSLSLSPGK;
>CP-3 chain 4
(SEQ ID NO: 120)
DIQMTQSPSSVSASVGDRVTITCKASQNVGSNVAWYQQKPGKSPKALIYSASYRY
SGVPSRFSGSGSGTDFTLTISSLQPEDFATYYCQQYNSYPYTFGGGTKVEIKGGGG
SSGAPRFLTRPKASVVSVGKDATLSCQIVGNPFPQVSWEKDKQPVTAGVRFRLAQ
DGDLYRLKILDLQLSDSGQYVCRARNAHGEAFACLGLQVDAEA.

In the present disclosure, fremanezumab was used as a positive control for the CGRP end, and LY-3451838 and ALDI-1910 were used as positive controls for the PACAP end.

### TEST EXAMPLES

### Test Example 1. ELISAs for Binding Activity of Chimeric Antibodies and Humanized Antibodies of C1 #, C9 #, C21 #, C28 #, and P14 #to CGRP, PACAP, and VIP

In this test example, the abilities of the chimeric antibodies and humanized antibodies of C1 #, C9 #, C21 #, and C28 #to bind to human CGRP and rat CGRP were assessed using ELISAs. Plates were coated with 1 Hg/mL human CGRP and rat CGRP, respectively, and blocked, and the antibodies, serially diluted, were then added. The plates were incubated for 1 h and washed, and a horseradish peroxidase-labeled anti-human Fc secondary antibody was then added. After incubation, color development was performed. The abilities of the chimeric antibody and humanized antibodies of P14 #to bind to PACAP38, PACAP27, and VIP were assessed using ELISAs. Plates were coated with 2 g/mL streptavidin and blocked, and biotinylated PACAP38, PACAP27, and VIP were then added to the plates, respectively. The plates were incubated for 1 h and washed, and the antibodies, serially diluted, were then added. The plates were incubated for 1 h and washed, and a horseradish peroxidase-labeled streptavidin secondary antibody was then added. After incubation, color development was performed. The EC₅₀ values (nM) for the binding of the antibodies to the corresponding antigens are shown in the table below.

TABLE 22
The binding activity of antibodies to CGRP

| | Human CGRP | Rat CGRP | | Human CGRP | Rat CGRP |
|---|---|---|---|---|---|
| Antibody | EC₅₀ (nM) | | Antibody | EC₅₀ (nM) | |
| C1H1L1 | 1.83 | 3.10 | C21#-CHI | 3.79 | 10.10 |
| C1H2L1 | 2.61 | 2.38 | C21H1L1 | 2.54 | 3.86 |
| C1H3L1 | 1.89 | 1.95 | C21H1L2 | 2.57 | 5.26 |
| C1H1L2 | 2.72 | 2.44 | C21H2L1 | 2.30 | 4.26 |
| C1H3L2 | 1.39 | 1.97 | C21H2L2 | 3.40 | 9.27 |
| C1#-CHI | 1.36 | 1.81 | C21H3L1 | 3.57 | 7.94 |
| C9H1L1 | 1.58 | 5.30 | C21H4L1 | 2.90 | 7.36 |
| C9H2L1 | 1.41 | 4.32 | C21H4L2 | 2.80 | 5.68 |
| C9H3L1 | 1.30 | 4.27 | C28H1L1 | 1.34 | 6.28 |
| C9H4L1 | 1.25 | 4.20 | C28H2L1 | 1.33 | 4.04 |
| C9H5L1 | 1.29 | 4.48 | C28H3L1 | 1.65 | 3.97 |
| C9H6L1 | 1.33 | 3.13 | C28H4L1 | 1.43 | 6.45 |
| C9H7L1 | 1.30 | 3.57 | C28H5L1 | 1.58 | 3.72 |
| C9H8L1 | 1.03 | 2.80 | C28H6L1 | 1.26 | 5.29 |
| C9#-CHI | 1.58 | 3.11 | C28H7L1 | 1.47 | 2.72 |
| | | | C28#-CHI | 1.29 | 7.40 |

TABLE 23
The binding activity of antibodies to PACAP and VIP

| | PACAP38 | PACAP27 | VIP |
|---|---|---|---|
| Antibody | EC₅₀ (nM) | | |
| P14#-CHI | 0.09 | 0.14 | N |
| P14H2L3 | 0.09 | 0.2 | N |
| P14H1L2 | 0.09 | 0.19 | N |
| P14H1L1 | 0.11 | 0.19 | N |
| ALD-1910 | 0.07 | 0.15 | 1.02 |
| LY-3451838 | 0.06 | 0.11 | 9.58 |

Note:
“N” means no binding.

The results show that the CGRP antibodies obtained by screening in the present disclosure all exhibited very good abilities to bind to CGRP and relatively good cross-binding activity to rat CGRP. The PACAP antibodies obtained by screening in the present disclosure all exhibited very good abilities to bind to PACAP38 and PACAP27 and did not bind to VIP, and their safety profiles were superior to those of the two positive antibodies.

### Test Example 2. Inhibition of Binding of PACAP38 to Receptors PAC1, VPAC1, and VPAC2 by Chimeric Antibodies and Humanized Antibodies of P14 #and P96 #

In this test example, the abilities of the chimeric antibodies and humanized antibodies of P14 #and P96 #to inhibit the binding of PACAP38 to the receptors PAC1, VPAC1, and VPAC2 were assessed using ELISAs. Plates were coated with 22 μg/mL PAC1, VPAC1, and VPAC, respectively, and blocked, and mixtures of the antibodies, serially diluted, and biotinylated PACAP38 were then added. The plates were incubated for 1 h and washed, and a horseradish peroxidase-labeled streptavidin secondary antibody was then added. After incubation, color development was performed. The IC₅₀ values (nM) for the inhibition of the binding of the antigen to the receptors by the antibodies are shown in the table below.

TABLE 24
The activity of antibodies in inhibiting
the binding of PACAP38 to receptors

| | PAC1 | VPAC1 | VPAC2 |
|---|---|---|---|
| Antibody | IC₅₀ (nM) | | |
| P14#-CHI | 0.48 | 0.27 | 0.23 |
| P14H2L3 | 0.67 | 0.58 | 0.4 |
| P14H1L2 | 0.45 | 0.19 | 0.18 |
| P14H1L1 | 0.51 | 0.2 | 0.21 |
| P96#-CHI | 0.83 | 0.53 | 0.68 |
| P96H1L1 | 0.76 | 0.53 | 0.61 |
| ALD-1910 | 4.89 | 0.65 | 1.58 |
| LY-3451838 | 6.46 | 1.34 | 1.42 |

The results show that the PACAP antibodies obtained by screening in the present disclosure all well inhibited the binding of PACAP38 to the receptors PAC1, VPAC1, and VPAC2.

### Test Example 3. Inhibition of cAMP Production in Cells by Chimeric Antibodies and Humanized Antibodies of C1 #, C9 #, C21 #, C28 #, P14 #, and P96 #

In this test example, the abilities of the chimeric antibodies and humanized antibodies of C1 #, C9 #, C21 #, and C28 #to inhibit cAMP production in SK-N-MC cells and the abilities of the chimeric antibodies and humanized antibodies of P14 #and P96 #to inhibit cAMP production in SH-SY5Y cells were assessed. The cells were incubated with the test antibodies at room temperature for half an hour. Subsequently, CGRP and PACAP38 were separately added as agonists, and the cells were incubated at room temperature for half an hour. Then cAMP-d2 and AnticAMP-Eu-Cryptate, prepared in cell lysis buffer, were separately added. After the cells were incubated at 30° C. in a dark place for 1 h, readings were taken. The IC50 (nM) values for the inhibition of cAMP production in the cells by the antibodies are shown in the table below.

TABLE 25
The activity of antibodies in inhibiting
cAMP production in SK-N-MC cells

| Antibody | IC₅₀ (nM) | Antibody | IC₅₀ (nM) |
|---|---|---|---|
| C1#-CHI | 7.96 | C21#-CHI | 1.28 |
| C1H3L1 | 9.77 | C21H2L1 | 1.25 |
| C9#-CHI | 20.96 | C28#-CHI | 1.21 |
| C9H8L1 | 12.23 | C28H6L1 | 2.49 |
| Fremanezumab | 40.03 | C28H2L1 | 2.55 |

TABLE 26
The activity of antibodies in inhibiting
cAMP production in SH-SY5Y cells

| Antibody | IC₅₀ (nM) | Antibody | IC₅₀ (nM) |
|---|---|---|---|
| P14#-CHI | 1.46 | P96#-CHI | 8.26 |
| P14H2L3 | 4.02 | P96H1L1 | 1.79 |
| P14H1L2 | 2.37 | | |

The results show that the CGRP and PACAP antibodies obtained by screening in the present disclosure all well inhibited cAMP production in the cells, and that their effects were superior to that of the control antibody fremanezumab.

### Test Example 4. Affinities of Antibodies for CGRPs of Different Species

The affinities of bispecific antibodies CP-1, CP-2, and CP-3 for human and rat CGRPs were assessed at 25° C. using a Biacore 8K system. First, the test antibodies (2 g/mL for the mAbs and 4 μg/mL for the diabodies), biotinylated (indicated by Biotin-), were each coupled onto an SA biosensor chip. Then samples of the antigen human CGRP were sequentially injected in ascending order for 180 s, followed by dissociation. After the test was finished, fitting was performed using a 1:1 binding model. The affinities of the antibodies for human CGRP are shown in the table below.

TABLE 27
The affinities of antibodies for human CGRP

| Antibody | ka (1/Ms) | kd (1/s) | KD (M) |
|---|---|---|---|
| Biotin-C21H2L1 | 5.04E+06 | 1.92E−05 | 3.81E−12 |
| Biotin-C28H6L1 | 5.03E+06 | 2.41E−05 | 4.79E−12 |
| Biotin-CP-1 | 2.48E+06 | 1.77E−05 | 7.15E−12 |
| Biotin-CP-2 | 2.00E+06 | 2.35E−05 | 1.17E−11 |
| Biotin-CP-3 | 4.34E+06 | 3.31E−05 | 7.64E−12 |

A Protein A biosensor chip was used to affinity-capture IgG, and a molecule sample was then allowed to flow through the surface of the chip. Reaction signals were detected in real time using a Biacore 8K system to obtain association and dissociation curves. After the dissociation in each experimental cycle was complete, the biosensor chip was washed and regenerated 3 times with 50 mM NaOH. Data fitting was performed using a 1:1 model. The affinities of the antibodies for rat CGRP are shown in Table 28.

TABLE 28
The affinities of antibodies for rat CGRP

| Antibody | ka (1/Ms) | kd (1/s) | KD (M) |
|---|---|---|---|
| C21H2L1 | 2.25E+06 | 2.32E−04 | 1.03E−10 |
| C28H6L1 | 2.06E+06 | 1.39E−04 | 6.74E−11 |
| CP-1 | 1.50E+06 | 1.16E−03 | 7.78E−10 |
| CP-2 | 2.07E+06 | 7.43E−04 | 3.58E−10 |
| CP-3 | 3.35E+06 | 3.01E−04 | 8.97E−11 |

### Test Example 5. Affinities of Antibodies for PACAP38 and PACAP27

In this test example, the abilities of bispecific antibodies CP-1, CP-2, and CP-3 to bind to PACAP38 and PACAP27 were assessed using ELISAs. Plates were coated with 1 μg/mL anti-human Fc and blocked, and the antibodies, serially diluted, were added. The plates were incubated for 1 h and washed, and biotinylated PACAP38 and PACAP27 were then added to the plates, respectively. The plates were incubated for 1 h and washed, and a horseradish peroxidase-labeled streptavidin secondary antibody was then added. After incubation, color development was performed. The EC₅₀ values (nM) for the binding of the antibodies to the corresponding antigens are shown in the table below. CP-1, CP-2, and CP-3 all exhibited relatively high affinities for PACAP38 and PACAP27.

TABLE 29
Assays for the abilities of antibodies
to bind to PACAP38 and PACAP27

| | |
|---|---|
| PACAP38 | PACAP27 |

| | |
|---|---|
| Antibody | EC₅₀ (nM) |

| | | |
|---|---|---|
| CP-1 | 0.15 | 0.28 |
| CP-2 | 0.1 | 0.16 |
| CP-3 | 0.22 | 0.33 |
| P14H2L3 | 0.13 | 0.16 |

### Test Example 6. Inhibition of cAMP Production in Cells by Bispecific Antibodies CP-1, CP-2, and CP-3

In this test example, the abilities of bispecific antibodies CP-1, CP-2, and CP-3 to inhibit cAMP production in SK-N-MC cells and SH-SY5Y cells were assessed. The cells were incubated with the test antibodies at room temperature for half an hour. Subsequently, CGRP and PACAP38 were separately added as agonists, and the cells were incubated at room temperature for half an hour. Then cAMP-d2 and AnticAMP-Eu-Cryptate, prepared in cell lysis buffer, were separately added. After the cells were incubated at 30° C. in a dark place for 1 h, readings were taken. The IC₅₀ values (nM) for the inhibition of cAMP production in the cells by the antibodies are shown in the table below. CP-1, CP-2, and CP-3 all well inhibited cAMP production in the cells.

TABLE 30
Assays for the abilities of antibodies
to inhibit cAMP production in cells

| | |
|---|---|
| SK-N-MC | SH-SY5Y |

| | |
|---|---|
| Antibody | IC₅₀ (nM) |

| | | |
|---|---|---|
| CP-1 | 2.66 | 4.31 |
| CP-2 | 2.03 | 1.92 |
| CP-3 | 5.79 | 10.99 |
| C21H2L1 | 1.98 | / |
| C28H6L1 | 1.61 | / |
| P14H2L3 | / | 2.4 |

### Test Example 7. Pharmacokinetic Evaluations of Bispecific Antibodies CP-1, CP-2, and CP-3 in Rats

Twenty-four male SD rats were divided into 6 groups. After intravenous (IV) administration and subcutaneous (SC) administration, whole blood was collected at different time points, and the concentrations of the bispecific antibodies and the IgG moiety in serum were determined using ELISAs. Their pharmacokinetic parameters and metabolic curves are shown in the table below.

TABLE 31
Pharmacokinetic parameters of bispecific antibodies

| | | IV-3mpk | | | SC-3mpk | | |
|---|---|---|---|---|---|---|---|
| Parameter | Target | CP-3 | CP-1 | CP-2 | CP-3 | CP-1 | CP-2 |
| t_{1/2} (d) | CGRP | 10.9 | 7.0 | 6.6 | 7.6 | 6.4 | 6.5 |
| | PACAP | 12.5 | 7.7 | 7.0 | 7.4 | 6.6 | 6.5 |
| AUC₀₋ₜ | CGRP | 5586 | 4670 | 4679 | 4001 | 3669 | 4536 |
| (μg/mL*h) | PACAP | 5749 | 4845 | 4388 | 4286 | 3851 | 4993 |
| | PACAP/CGRP | 103% | 104% | 94% | 107% | 105% | 110% |
| Tmax (h) | CGRP | / | / | / | 54.0 | 84.0 | 64.0 |
| | PACAP | / | / | / | 54.0 | 72.0 | 64.0 |
| Bioavailability, F | CGRP | 0.72 | 0.79 | 0.97 | / | / | / |
| | PACAP | 0.75 | 0.79 | 1.14 | / | / | / |

Note:
“/”means no detection.

The results show that the bispecific molecules of the present disclosure exhibited no significant difference in half-life between intravenous administration and subcutaneous administration in rats, and that they all exhibited relatively long half-lives, relatively high bioavailability, and relatively good molecular integrity and stability, demonstrating excellent pharmacokinetic profiles.

### Biological Evaluations of In Vivo Activity

### Test Example 8. PACAP38-Induced Mouse Model of cAMP Elevation

In this test example, the in vivo inhibitory pharmacodynamic effects of the test antibodies on mouse plasma cAMP elevation were assessed using a PACAP38-induced mouse model of plasma cAMP elevation.

Experimental animals were randomly grouped by body weight, and each was intraperitoneally given an antibody or PBS at 10 mL/kg. Twenty-four hours after the administration, a PACAP38-rolipram solution or a PBS-rolipram solution (control group) was injected into the tail vein at 5 mL/kg. Ten minutes after PACAP38 was administered, blood was collected and anticoagulated with EDTA. Subsequently, the cAMP level in plasma was determined. The inhibitory pharmacodynamic effects of the antibodies on mouse plasma cAMP elevation are shown in the table below. The results show that CP-1, CP-2, and CP-3 inhibited cAMP production at both high and low doses compared to the blank group.

TABLE 32
The efficacy of bispecific antibodies in
an induced mouse model of cAMP elevation

| Group | Dose | cAMP inhibition rate (%) |
|---|---|---|
| PBS | / | 0 |
| CP-2 | 1.3 mpk | 34 |
| CP-2 | 4 mpk | 42 |
| CP-3 | 1 mpk | 26 |
| CP-3 | 3 mpk | 43 |
| CP-1 | 4 mpk | 64 |
| CP-1 | 1.3 mpk | 22 |
| P14H2L3 | 1 mpk | 24 |
| P14H2L3 | 3 mpk | 41 |

Note:
“/” means no administration.

### Test Example 9. Evaluations of Efficacy of Bispecific Antibodies CP-1, CP-2, and CP-3 in Capsaicin-Induced Rat Model of Cutaneous Blood Flow

In this test example, the effects of CP-1, CP-2, and CP-3 on blood flow were studied using a capsaicin-induced SD rat model of cutaneous vasodilation.

One O-shaped mark with a diameter of about 6 mm was drawn on each side of the ventral midline of a rat, avoiding regions with clearly visible blood vessels. After the baseline cutaneous blood flow rates within the O-shaped marks were recorded, 4 L of capsaicin solution was uniformly applied within a mark, and cutaneous blood flow rate data within the O-shaped marks were collected at different time points. The blood flow increase percentage (%) at each detection point was calculated based on the blood flow value at each time point (blood flow t): blood flow increase percentage (%)=(blood flow t−blood flow baseline)/blood flow baseline×100, and the overall blood flow increase rate (%) was calculated: overall blood flow increase rate (%)=blood flow increase percentage on capsaicin-treated side−blood flow increase percentage on control side. The blood flow rate increase inhibition rate (%) of each group was calculated based on the overall blood flow increase rate: blood flow rate increase inhibition rate (%)=(average overall blood flow increase rate of model control group−overall blood flow increase rate of treatment group)/average overall blood flow increase rate of model control group×100. The overall blood flow increase rates and blood flow increase inhibition rates for the antibodies are shown in the table below.

TABLE 33
Overall blood flow increase rates and
blood flow increase inhibition rates

| | | Overall blood flow | Blood flow increase |
|---|---|---|---|
| Group | Dose | increase rate (%) | inhibition rate (%) |
| PBS | / | 44.6 | No detection |
| CP-2 | 0.4 mpk | 11.8 | 73.6 |
| CP-2 | 2 mpk | 10.8 | 75.8 |
| CP-3 | 1.5 mpk | 16.6 | 62.9 |
| CP-1 | 2 mpk | 13.5 | 69.8 |
| C21H2L1 | 1.5 mpk | 24.7 | 46.2 |
| Fremanezumab | 1.5 mpk | 42 | 5.9 |

Note:
“/” means no administration.

The results show that CP-3, CP-2, and CP-1 exhibited significant inhibitory effects on capsaicin-induced blood flow increases. Under comparable dose conditions, the activity of CP-2 was significantly better than that of monospecific antibody C21H2L1, suggesting that the CGRP/PACAP bispecific antibody has a synergistic effect.

Although the above invention has been described in detail using drawings and examples for a clear understanding, the description and examples should not be construed as limiting the scope of the present disclosure. The disclosures of all the patents and scientific documents cited herein are clearly incorporated by reference in their entirety.

1. An antigen-binding molecule that specifically binds to CGRP and PACAP, comprising at least one antigen-binding moiety that specifically binds to CGRP and at least one antigen-binding moiety that specifically binds to PACAP, wherein the antigen-binding moiety that specifically binds to CGRP comprises a heavy chain variable region CGRP-VH and a light chain variable region CGRP-VL, and the antigen-binding moiety that specifically binds to PACAP comprises a heavy chain variable region PACAP-VH and a light chain variable region PACAP-VL;
preferably,
the antigen-binding molecule binds to human CGRP at 25° C. with a KD of less than 1×10⁻¹⁰ M, as measured by a surface plasmon resonance assay, and/or
the antigen-binding molecule binds to rat CGRP at 25° C. with a KD of less than 1×10⁻⁹ M, as measured by a surface plasmon resonance assay, and/or
the antigen-binding molecule binds to PACAP38 and PACAP27 with an EC₅₀ of less than 1×10⁻⁹ M, as measured by an ELISA, and/or
the antigen-binding molecule does not bind to VIP, and/or
the antigen-binding molecule inhibits cAMP production in cells with an IC₅₀ of less than 4×10⁻⁸ M under conditions of CGRP induction, and/or
the antigen-binding molecule inhibits cAMP production in cells with an IC₅₀ of less than 2×10⁻⁸ M under conditions of PACAP induction.

2. The antigen-binding molecule according to claim 1, wherein:
(i) a CGRP-HCDR1, a CGRP-HCDR2, and a CGRP-HCDR3 in the CGRP-VH comprise the amino acid sequences of a CGRP-HCDR1, a CGRP-HCDR2, and a CGRP-HCDR3 in SEQ ID NO: 83, 46, 76, 77, 78, 79, 80, 81, or 82, respectively, and
a CGRP-LCDR1, a CGRP-LCDR2, and a CGRP-LCDR3 in the CGRP-VL comprise the amino acid sequences of a CGRP-LCDR1, a CGRP-LCDR2, and a CGRP-LCDR3 in SEQ ID NO: 85, 47, or 84, respectively, or
(ii) a CGRP-HCDR1, a CGRP-HCDR2, and a CGRP-HCDR3 in the CGRP-VH comprise the amino acid sequences of a CGRP-HCDR1, a CGRP-HCDR2, and a CGRP-HCDR3 in SEQ ID NO: 72, 44, 68, 69, 70, or 71, respectively, and
a CGRP-LCDR1, a CGRP-LCDR2, and a CGRP-LCDR3 in the CGRP-VL comprise the amino acid sequences of a CGRP-LCDR1, a CGRP-LCDR2, and a CGRP-LCDR3 in SEQ ID NO: 75, 45, 73, or 74, respectively, or
(iii) a CGRP-HCDR1, a CGRP-HCDR2, and a CGRP-HCDR3 in the CGRP-VH comprise the amino acid sequences of a CGRP-HCDR1, a CGRP-HCDR2, and a CGRP-HCDR3 in SEQ ID NO: 40, 54, 55, or 56, respectively, and
a CGRP-LCDR1, a CGRP-LCDR2, and a CGRP-LCDR3 in the CGRP-VL comprise the amino acid sequences of a CGRP-LCDR1, a CGRP-LCDR2, and a CGRP-LCDR3 in SEQ ID NO: 41, 57, or 58, respectively, or
(iv) a CGRP-HCDR1, a CGRP-HCDR2, and a CGRP-HCDR3 in the CGRP-VH comprise the amino acid sequences of a CGRP-HCDR1, a CGRP-HCDR2, and a CGRP-HCDR3 in SEQ ID NO: 42, 59, 60, 61, 62, 63, 64, 65, or 66, respectively, and
a CGRP-LCDR1, a CGRP-LCDR2, and a CGRP-LCDR3 in the CGRP-VL comprise the amino acid sequences of a CGRP-LCDR1, a CGRP-LCDR2, and a CGRP-LCDR3 in SEQ ID NO: 43 or 67, respectively;
preferably,
(i) the CGRP-VH comprises: a CGRP-HCDR1 comprising the amino acid sequence of SEQ ID NO: 22, a CGRP-HCDR2 comprising the amino acid sequence of SEQ ID NO: 106, 23, 103, 104, or 105, and a CGRP-HCDR3 comprising the amino acid sequence of SEQ ID NO: 24, and
the CGRP-VL comprises: a CGRP-LCDR1 comprising the amino acid sequence of SEQ ID NO: 25, a CGRP-LCDR2 comprising the amino acid sequence of SEQ ID NO: 26, and a CGRP-LCDR3 comprising the amino acid sequence of SEQ ID NO: 27, or
(ii) the CGRP-VH comprises: a CGRP-HCDR1 comprising the amino acid sequence of SEQ ID NO: 16, a CGRP-HCDR2 comprising the amino acid sequence of SEQ ID NO: 101, 17, 100, 102, or 192, and a CGRP-HCDR3 comprising the amino acid sequence of SEQ ID NO: 18, and
the CGRP-VL comprises: a CGRP-LCDR1 comprising the amino acid sequence of SEQ ID NO: 19, a CGRP-LCDR2 comprising the amino acid sequence of SEQ ID NO: 20, and a CGRP-LCDR3 comprising the amino acid sequence of SEQ ID NO: 21, or
(iii) the CGRP-VH comprises: a CGRP-HCDR1 comprising the amino acid sequence of SEQ ID NO: 4, a CGRP-HCDR2 comprising the amino acid sequence of SEQ ID NO: 5, 93, 94, or 95, and a CGRP-HCDR3 comprising the amino acid sequence of SEQ ID NO: 6, and
the CGRP-VL comprises: a CGRP-LCDR1 comprising the amino acid sequence of SEQ ID NO: 7, a CGRP-LCDR2 comprising the amino acid sequence of SEQ ID NO: 8, and a CGRP-LCDR3 comprising the amino acid sequence of SEQ ID NO: 9, or
(iv) the CGRP-VH comprises: a CGRP-HCDR1 comprising the amino acid sequence of SEQ ID NO: 10, a CGRP-HCDR2 comprising the amino acid sequence of SEQ ID NO: 11, 96, 97, 98, or 99, and a CGRP-HCDR3 comprising the amino acid sequence of SEQ ID NO: 12, and
the CGRP-VL comprises: a CGRP-LCDR1 comprising the amino acid sequence of SEQ ID NO: 13, a CGRP-LCDR2 comprising the amino acid sequence of SEQ ID NO: 14, and a CGRP-LCDR3 comprising the amino acid sequence of SEQ ID NO: 15;
more preferably,
the CGRP-VH comprises: a CGRP-HCDR1 comprising the amino acid sequence of SEQ ID NO: 22, a CGRP-HCDR2 comprising the amino acid sequence of SEQ ID NO: 106, and a CGRP-HCDR3 comprising the amino acid sequence of SEQ ID NO: 24, and
the CGRP-VL comprises: a CGRP-LCDR1 comprising the amino acid sequence of SEQ ID NO: 25, a CGRP-LCDR2 comprising the amino acid sequence of SEQ ID NO: 26, and a CGRP-LCDR3 comprising the amino acid sequence of SEQ ID NO: 27, or
the CGRP-VH comprises: a CGRP-HCDR1 comprising the amino acid sequence of SEQ ID NO: 16, a CGRP-HCDR2 comprising the amino acid sequence of SEQ ID NO: 101, and a CGRP-HCDR3 comprising the amino acid sequence of SEQ ID NO: 18, and
the CGRP-VL comprises: a CGRP-LCDR1 comprising the amino acid sequence of SEQ ID NO: 19, a CGRP-LCDR2 comprising the amino acid sequence of SEQ ID NO: 20, and a CGRP-LCDR3 comprising the amino acid sequence of SEQ ID NO: 21.

3. The antigen-binding molecule according to claim 2, wherein:
(i) the CGRP-VH comprises the amino acid sequence of SEQ ID NO: 83, 46, 76, 77, 78, 79, 80, 81, or 82, and the CGRP-VL comprises the amino acid sequence of SEQ ID NO: 85, 47, or 84, or
(ii) the CGRP-VH comprises the amino acid sequence of SEQ ID NO: 72, 44, 68, 69, 70, or 71, and the CGRP-VL comprises the amino acid sequence of SEQ ID NO: 75, 45, 73, or 74, or
(iii) the CGRP-VH comprises the amino acid sequence of SEQ ID NO: 40, 54, 55, or 56, and the CGRP-VL comprises the amino acid sequence of SEQ ID NO: 41, 57, or 58, or
(iv) the CGRP-VH comprises the amino acid sequence of SEQ ID NO: 42, 59, 60, 61, 62, 63, 64, 65, or 66, and the CGRP-VL comprises the amino acid sequence of SEQ ID NO: 43 or 67;
preferably,
(i) the CGRP-VH comprises the amino acid sequence of SEQ ID NO: 83, and the CGRP-VL comprises the amino acid sequence of SEQ ID NO: 85, or
the CGRP-VH comprises the amino acid sequence of SEQ ID NO: 46, and the CGRP-VL comprises the amino acid sequence of SEQ ID NO: 47, or
the CGRP-VH comprises the amino acid sequence of SEQ ID NO: 76, and the CGRP-VL comprises the amino acid sequence of SEQ ID NO: 84, or
the CGRP-VH comprises the amino acid sequence of SEQ ID NO: 77, and the CGRP-VL comprises the amino acid sequence of SEQ ID NO: 84, or
the CGRP-VH comprises the amino acid sequence of SEQ ID NO: 78, and the CGRP-VL comprises the amino acid sequence of SEQ ID NO: 84, or
the CGRP-VH comprises the amino acid sequence of SEQ ID NO: 79, and the CGRP-VL comprises the amino acid sequence of SEQ ID NO: 84, or
the CGRP-VH comprises the amino acid sequence of SEQ ID NO: 80, and the CGRP-VL comprises the amino acid sequence of SEQ ID NO: 84, or
the CGRP-VH comprises the amino acid sequence of SEQ ID NO: 81, and the CGRP-VL comprises the amino acid sequence of SEQ ID NO: 84, or
the CGRP-VH comprises the amino acid sequence of SEQ ID NO: 82, and the CGRP-VL comprises the amino acid sequence of SEQ ID NO: 84; or
(ii) the CGRP-VH comprises the amino acid sequence of SEQ ID NO: 72, and the CGRP-VL comprises the amino acid sequence of SEQ ID NO: 75, or
the CGRP-VH comprises the amino acid sequence of SEQ ID NO: 44, and the CGRP-VL comprises the amino acid sequence of SEQ ID NO: 45, or
the CGRP-VH comprises the amino acid sequence of SEQ ID NO: 68, and the CGRP-VL comprises the amino acid sequence of SEQ ID NO: 73, or
the CGRP-VH comprises the amino acid sequence of SEQ ID NO: 68, and the CGRP-VL comprises the amino acid sequence of SEQ ID NO: 74, or
the CGRP-VH comprises the amino acid sequence of SEQ ID NO: 69, and the CGRP-VL comprises the amino acid sequence of SEQ ID NO: 73, or
the CGRP-VH comprises the amino acid sequence of SEQ ID NO: 69, and the CGRP-VL comprises the amino acid sequence of SEQ ID NO: 74, or
the CGRP-VH comprises the amino acid sequence of SEQ ID NO: 70, and the CGRP-VL comprises the amino acid sequence of SEQ ID NO: 73, or
the CGRP-VH comprises the amino acid sequence of SEQ ID NO: 71, and the CGRP-VL comprises the amino acid sequence of SEQ ID NO: 73, or
the CGRP-VH comprises the amino acid sequence of SEQ ID NO: 71, and the CGRP-VL comprises the amino acid sequence of SEQ ID NO: 74; or
(iii) the CGRP-VH comprises the amino acid sequence of SEQ ID NO: 40, and the CGRP-VL comprises the amino acid sequence of SEQ ID NO: 41, or
the CGRP-VH comprises the amino acid sequence of SEQ ID NO: 54, and the CGRP-VL comprises the amino acid sequence of SEQ ID NO: 57, or
the CGRP-VH comprises the amino acid sequence of SEQ ID NO: 55, and the CGRP-VL comprises the amino acid sequence of SEQ ID NO: 57, or
the CGRP-VH comprises the amino acid sequence of SEQ ID NO: 56, and the CGRP-VL comprises the amino acid sequence of SEQ ID NO: 57, or
the CGRP-VH comprises the amino acid sequence of SEQ ID NO: 54, and the CGRP-VL comprises the amino acid sequence of SEQ ID NO: 58, or
the CGRP-VH comprises the amino acid sequence of SEQ ID NO: 56, and the CGRP-VL comprises the amino acid sequence of SEQ ID NO: 58; or
(iv) the CGRP-VH comprises the amino acid sequence of SEQ ID NO: 42, and the CGRP-VL comprises the amino acid sequence of SEQ ID NO: 43, or
the CGRP-VH comprises the amino acid sequence of SEQ ID NO: 59, and the CGRP-VL comprises the amino acid sequence of SEQ ID NO: 67, or
the CGRP-VH comprises the amino acid sequence of SEQ ID NO: 60, and the CGRP-VL comprises the amino acid sequence of SEQ ID NO: 67, or
the CGRP-VH comprises the amino acid sequence of SEQ ID NO: 61, and the CGRP-VL comprises the amino acid sequence of SEQ ID NO: 67, or
the CGRP-VH comprises the amino acid sequence of SEQ ID NO: 62, and the CGRP-VL comprises the amino acid sequence of SEQ ID NO: 67, or
the CGRP-VH comprises the amino acid sequence of SEQ ID NO: 63, and the CGRP-VL comprises the amino acid sequence of SEQ ID NO: 67, or
the CGRP-VH comprises the amino acid sequence of SEQ ID NO: 64, and the CGRP-VL comprises the amino acid sequence of SEQ ID NO: 67, or
the CGRP-VH comprises the amino acid sequence of SEQ ID NO: 65, and the CGRP-VL comprises the amino acid sequence of SEQ ID NO: 67, or
the CGRP-VH comprises the amino acid sequence of SEQ ID NO: 66, and the CGRP-VL comprises the amino acid sequence of SEQ ID NO: 67;
more preferably,
(i) the CGRP-VH comprises the amino acid sequence of SEQ ID NO: 83, and the CGRP-VL comprises the amino acid sequence of SEQ ID NO: 85, or
the CGRP-VH comprises the amino acid sequence of SEQ ID NO: 81, and the CGRP-VL comprises the amino acid sequence of SEQ ID NO: 84; or
(ii) the CGRP-VH comprises the amino acid sequence of SEQ ID NO: 72, and the CGRP-VL comprises the amino acid sequence of SEQ ID NO: 75.

4. The antigen-binding molecule according to claim 1, wherein:
(v) a PACAP-HCDR1, a PACAP-HCDR2, and a PACAP-HCDR3 in the PACAP-VH comprise the amino acid sequences of a PACAP-HCDR1, a PACAP-HCDR2, and a PACAP-HCDR3 in SEQ ID NO: 87, 48, or 86, respectively, and
a PACAP-LCDR1, a PACAP-LCDR2, and a PACAP-LCDR3 in the PACAP-VL comprise the amino acid sequences of a PACAP-LCDR1, a PACAP-LCDR2, and a PACAP-LCDR3 in SEQ ID NO: 90, 49, 88, or 89, respectively, or
(vi) a PACAP-HCDR1, a PACAP-HCDR2, and a PACAP-HCDR3 in the PACAP-VH comprise the amino acid sequences of a PACAP-HCDR1, a PACAP-HCDR2, and a PACAP-HCDR3 in SEQ ID NO: 50 or 91, respectively, and
a PACAP-LCDR1, a PACAP-LCDR2, and a PACAP-LCDR3 in the PACAP-VL comprise the amino acid sequences of a PACAP-LCDR1, a PACAP-LCDR2, and a PACAP-LCDR3 in SEQ ID NO: 51 or 92, respectively;
preferably,
(v) the PACAP-VH comprises: a PACAP-HCDR1 comprising the amino acid sequence of SEQ ID NO: 28, a PACAP-HCDR2 comprising the amino acid sequence of SEQ ID NO: 29, and a PACAP-HCDR3 comprising the amino acid sequence of SEQ ID NO: 107 or 30, and
the PACAP-VL comprises: a PACAP-LCDR1 comprising the amino acid sequence of SEQ ID NO: 31, a PACAP-LCDR2 comprising the amino acid sequence of SEQ ID NO: 108 or 32, and a PACAP-LCDR3 comprising the amino acid sequence of SEQ ID NO: 33; or
(vi) the PACAP-VH comprises: a PACAP-HCDR1 comprising the amino acid sequence of SEQ ID NO: 34, a PACAP-HCDR2 comprising the amino acid sequence of SEQ ID NO: 35 or 109, and a PACAP-HCDR3 comprising the amino acid sequence of SEQ ID NO: 36, and
the PACAP-VL comprises: a PACAP-LCDR1 comprising the amino acid sequence of SEQ ID NO: 37, a PACAP-LCDR2 comprising the amino acid sequence of SEQ ID NO: 38, and a PACAP-LCDR3 comprising the amino acid sequence of SEQ ID NO: 39;
more preferably,
the PACAP-VH comprises: a PACAP-HCDR1 comprising the amino acid sequence of SEQ ID NO: 28, a PACAP-HCDR2 comprising the amino acid sequence of SEQ ID NO: 29, and a PACAP-HCDR3 comprising the amino acid sequence of SEQ ID NO: 107, and
the PACAP-VL comprises: a PACAP-LCDR1 comprising the amino acid sequence of SEQ ID NO: 31, a PACAP-LCDR2 comprising the amino acid sequence of SEQ ID NO: 108, and a PACAP-LCDR3 comprising the amino acid sequence of SEQ ID NO: 33.

5. The antigen-binding molecule according to claim 4, wherein:
(v) the PACAP-VH comprises the amino acid sequence of SEQ ID NO: 87, 48, or 86, and the PACAP-VL comprises the amino acid sequence of SEQ ID NO: 90, 49, 88, or 89, or
(vi) the PACAP-VH comprises the amino acid sequence of SEQ ID NO: 50 or 91, and the PACAP-VL comprises the amino acid sequence of SEQ ID NO: 51 or 92;
preferably,
(v) the PACAP-VH comprises the amino acid sequence of SEQ ID NO: 87, and the PACAP-VL comprises the amino acid sequence of SEQ ID NO: 90, or
the PACAP-VH comprises the amino acid sequence of SEQ ID NO: 48, and the PACAP-VL comprises the amino acid sequence of SEQ ID NO: 49, or
the PACAP-VH comprises the amino acid sequence of SEQ ID NO: 86, and the PACAP-VL comprises the amino acid sequence of SEQ ID NO: 88, or
the PACAP-VH comprises the amino acid sequence of SEQ ID NO: 86, and the PACAP-VL comprises the amino acid sequence of SEQ ID NO: 89; or
(vi) the PACAP-VH comprises the amino acid sequence of SEQ ID NO: 50, and the PACAP-VL comprises the amino acid sequence of SEQ ID NO: 51, or
the PACAP-VH comprises the amino acid sequence of SEQ ID NO: 91, and the PACAP-VL comprises the amino acid sequence of SEQ ID NO: 92;
more preferably,
(v) the PACAP-VH comprises the amino acid sequence of SEQ ID NO: 87, and the PACAP-VL comprises the amino acid sequence of SEQ ID NO: 90.

6. The antigen-binding molecule according to claim 1, wherein:
the CGRP-VH comprises: a CGRP-HCDR1 comprising the amino acid sequence of SEQ ID NO: 22, a CGRP-HCDR2 comprising the amino acid sequence of SEQ ID NO: 106, and a CGRP-HCDR3 comprising the amino acid sequence of SEQ ID NO: 24, and the CGRP-VL comprises: a CGRP-LCDR1 comprising the amino acid sequence of SEQ ID NO: 25, a CGRP-LCDR2 comprising the amino acid sequence of SEQ ID NO: 26, and a CGRP-LCDR3 comprising the amino acid sequence of SEQ ID NO: 27, or
the CGRP-VH comprises: a CGRP-HCDR1 comprising the amino acid sequence of SEQ ID NO: 16, a CGRP-HCDR2 comprising the amino acid sequence of SEQ ID NO: 101, and a CGRP-HCDR3 comprising the amino acid sequence of SEQ ID NO: 18, and the CGRP-VL comprises: a CGRP-LCDR1 comprising the amino acid sequence of SEQ ID NO: 19, a CGRP-LCDR2 comprising the amino acid sequence of SEQ ID NO: 20, and a CGRP-LCDR3 comprising the amino acid sequence of SEQ ID NO: 21; and
the PACAP-VH comprises: a PACAP-HCDR1 comprising the amino acid sequence of SEQ ID NO: 28, a PACAP-HCDR2 comprising the amino acid sequence of SEQ ID NO: 29, and a PACAP-HCDR3 comprising the amino acid sequence of SEQ ID NO: 107, and the PACAP-VL comprises: a PACAP-LCDR1 comprising the amino acid sequence of SEQ ID NO: 31, a PACAP-LCDR2 comprising the amino acid sequence of SEQ ID NO: 108, and a PACAP-LCDR3 comprising the amino acid sequence of SEQ ID NO: 33;
preferably,
the CGRP-VH comprises the amino acid sequence of SEQ ID NO: 83, and the CGRP-VL comprises the amino acid sequence of SEQ ID NO: 85, or
the CGRP-VH comprises the amino acid sequence of SEQ ID NO: 81, and the CGRP-VL comprises the amino acid sequence of SEQ ID NO: 84, or
the CGRP-VH comprises the amino acid sequence of SEQ ID NO: 72, and the CGRP-VL comprises the amino acid sequence of SEQ ID NO: 75; and
the PACAP-VH comprises the amino acid sequence of SEQ ID NO: 87, and the PACAP-VL comprises the amino acid sequence of SEQ ID NO: 90.

7. The antigen-binding molecule according to claim 1, wherein the antigen-binding molecule further comprises an Fc region; preferably, the Fc region is an IgG Fc region; more preferably, the Fc region is an IgG1 Fc region;
preferably, the Fc region comprises one or more amino acid substitutions capable of increasing the serum half-life of the antigen-binding molecule;
more preferably, the Fc region is a human IgG1 Fc region, with the amino acid residue at position 252 being Y, the amino acid residue at position 254 being T, and the amino acid residue at position 256 being E, as numbered according to the EU index.

8. The antigen-binding molecule according to claim 1, wherein the antigen-binding molecule comprises:
two antigen-binding moieties that specifically bind to CGRP,
two antigen-binding moieties that specifically bind to PACAP, and
an Fc region;
preferably,
the antigen-binding molecule comprises:
two first chains having a structure represented by formula (a), and
two second chains having a structure represented by formula (b):
[PACAP-VH]-[CH1]-[CGRP-VH]-[linker 1]-[CGRP-VL]-[linker 2]-[one subunit of the Fc region], and formula (a)
[PACAP-VL]-[CL], formula (b)
wherein the structures represented by formulas (a) and (b) are arranged from the N-terminus to the C-terminus, and the linker 1 and the linker 2 are identical or different peptide linkers;
more preferably,
the antigen-binding molecule comprises: two first chains comprising the amino acid sequence of SEQ ID NO: 113 and two second chains comprising the amino acid sequence of SEQ ID NO: 114;
or
the antigen-binding molecule comprises: two first chains comprising the amino acid sequence of SEQ ID NO: 194 and two second chains comprising the amino acid sequence of SEQ ID NO: 114;
or
the antigen-binding molecule comprises: two first chains comprising the amino acid sequence of SEQ ID NO: 111 and two second chains comprising the amino acid sequence of SEQ ID NO: 112.

9. The antigen-binding molecule according to claim 1, wherein the antigen-binding molecule comprises:
one antigen-binding moiety that specifically binds to CGRP,
one antigen-binding moiety that specifically binds to PACAP, and
an Fc region,
wherein the Fc region comprises a first subunit Fc1 and a second subunit Fc2 that are capable of associating with each other, and the Fc1 and Fc2 each independently have one or more amino acid substitutions that reduce homodimerization of the Fc region;
preferably,
the Fc1 comprises a knob structure according to the knob-into-hole technique, and the Fc2 comprises a hole structure according to the knob-into-hole technique;
more preferably,
the amino acid residue at position 366 of the Fc1 is W; and
the amino acid residue at position 366 of the Fc2 is S, the amino acid residue at position 368 is A, and the amino acid residue at position 407 is V, as numbered according to the EU index.

10. The antigen-binding molecule according to claim 9, wherein one of the antigen-binding moiety that specifically binds to CGRP and the antigen-binding moiety that specifically binds to PACAP comprises a Titin chain and an Obscurin chain that are capable of forming a dimer;
preferably,
the Titin chain comprises an amino acid sequence selected from the group consisting of SEQ ID NO: 121 to SEQ ID NO: 139, and
the Obscurin chain comprises an amino acid sequence selected from the group consisting of SEQ ID NO: 140 to SEQ ID NO: 180;
more preferably,
the Titin chain comprises the amino acid sequence of SEQ ID NO: 137, and the Obscurin chain comprises the amino acid sequence of SEQ ID NO: 175.

11. The antigen-binding molecule according to claim 10, wherein the antigen-binding molecule comprises:
one first chain having a structure represented by formula (c),
one second chain having a structure represented by formula (b),
one third chain having a structure represented by formula (d), and
one fourth chain having a structure represented by formula (e):
[PACAP-VH]-[CH1]-[Fc1], formula (c)
[PACAP-VL]-[CL], formula (b)
[CGRP-VH]-[linker 3]-[Titin chain]-[Fc2], and formula (d)
[CGRP-VL]-[linker 4]-[Obscurin chain], formula (e)
wherein the structures represented by formulas (c), (b), (d), and (e) are arranged from the N-terminus to the C-terminus, and the linker 3 and the linker 4 are identical or different peptide linkers;
preferably,
the antigen-binding molecule comprises: a first chain comprising the amino acid sequence of SEQ ID NO: 117, a second chain comprising the amino acid sequence of SEQ ID NO: 118, a third chain comprising the amino acid sequence of SEQ ID NO: 119, and a fourth chain comprising the amino acid sequence of SEQ ID NO: 120.

12. An isolated antibody capable of specifically binding to CGRP, comprising a heavy chain variable region CGRP-VH and a light chain variable region CGRP-VL, wherein:
(i) a CGRP-HCDR1, a CGRP-HCDR2, and a CGRP-HCDR3 in the CGRP-VH comprise the amino acid sequences of a CGRP-HCDR1, a CGRP-HCDR2, and a CGRP-HCDR3 in SEQ ID NO: 83, 46, 76, 77, 78, 79, 80, 81, or 82, respectively, and a CGRP-LCDR1, a CGRP-LCDR2, and a CGRP-LCDR3 in the CGRP-VL comprise the amino acid sequences of a CGRP-LCDR1, a CGRP-LCDR2, and a CGRP-LCDR3 in SEQ ID NO: 85, 47, or 84, respectively, or
(ii) a CGRP-HCDR1, a CGRP-HCDR2, and a CGRP-HCDR3 in the CGRP-VH comprise the amino acid sequences of a CGRP-HCDR1, a CGRP-HCDR2, and a CGRP-HCDR3 in SEQ ID NO: 72, 44, 68, 69, 70, or 71, respectively, and a CGRP-LCDR1, a CGRP-LCDR2, and a CGRP-LCDR3 in the CGRP-VL comprise the amino acid sequences of a CGRP-LCDR1, a CGRP-LCDR2, and a CGRP-LCDR3 in SEQ ID NO: 75, 45, 73, or 74, respectively, or
(iii) a CGRP-HCDR1, a CGRP-HCDR2, and a CGRP-HCDR3 in the CGRP-VH comprise the amino acid sequences of a CGRP-HCDR1, a CGRP-HCDR2, and a CGRP-HCDR3 in SEQ ID NO: 40, 54, 55, or 56, respectively, and a CGRP-LCDR1, a CGRP-LCDR2, and a CGRP-LCDR3 in the CGRP-VL comprise the amino acid sequences of a CGRP-LCDR1, a CGRP-LCDR2, and a CGRP-LCDR3 in SEQ ID NO: 41, 57, or 58, respectively, or
(iv) a CGRP-HCDR1, a CGRP-HCDR2, and a CGRP-HCDR3 in the CGRP-VH comprise the amino acid sequences of a CGRP-HCDR1, a CGRP-HCDR2, and a CGRP-HCDR3 in SEQ ID NO: 42, 59, 60, 61, 62, 63, 64, 65, or 66, respectively, and a CGRP-LCDR1, a CGRP-LCDR2, and a CGRP-LCDR3 in the CGRP-VL comprise the amino acid sequences of a CGRP-LCDR1, a CGRP-LCDR2, and a CGRP-LCDR3 in SEQ ID NO: 43 or 67, respectively;
preferably,
(i) the CGRP-VH comprises: a CGRP-HCDR1 comprising the amino acid sequence of SEQ ID NO: 22, a CGRP-HCDR2 comprising the amino acid sequence of SEQ ID NO: 106, 23, 103, 104, or 105, and a CGRP-HCDR3 comprising the amino acid sequence of SEQ ID NO: 24, and the CGRP-VL comprises: a CGRP-LCDR1 comprising the amino acid sequence of SEQ ID NO: 25, a CGRP-LCDR2 comprising the amino acid sequence of SEQ ID NO: 26, and a CGRP-LCDR3 comprising the amino acid sequence of SEQ ID NO: 27, or
(ii) the CGRP-VH comprises: a CGRP-HCDR1 comprising the amino acid sequence of SEQ ID NO: 16, a CGRP-HCDR2 comprising the amino acid sequence of SEQ ID NO: 101, 17, 100, 102, or 192, and a CGRP-HCDR3 comprising the amino acid sequence of SEQ ID NO: 18, and the CGRP-VL comprises: a CGRP-LCDR1 comprising the amino acid sequence of SEQ ID NO: 19, a CGRP-LCDR2 comprising the amino acid sequence of SEQ ID NO: 20, and a CGRP-LCDR3 comprising the amino acid sequence of SEQ ID NO: 21, or
(iii) the CGRP-VH comprises: a CGRP-HCDR1 comprising the amino acid sequence of SEQ ID NO: 4, a CGRP-HCDR2 comprising the amino acid sequence of SEQ ID NO: 5, 93, 94, or 95, and a CGRP-HCDR3 comprising the amino acid sequence of SEQ ID NO: 6, and the CGRP-VL comprises: a CGRP-LCDR1 comprising the amino acid sequence of SEQ ID NO: 7, a CGRP-LCDR2 comprising the amino acid sequence of SEQ ID NO: 8, and a CGRP-LCDR3 comprising the amino acid sequence of SEQ ID NO: 9, or
(iv) the CGRP-VH comprises: a CGRP-HCDR1 comprising the amino acid sequence of SEQ ID NO: 10, a CGRP-HCDR2 comprising the amino acid sequence of SEQ ID NO: 11, 96, 97, 98, or 99, and a CGRP-HCDR3 comprising the amino acid sequence of SEQ ID NO: 12, and the CGRP-VL comprises: a CGRP-LCDR1 comprising the amino acid sequence of SEQ ID NO: 13, a CGRP-LCDR2 comprising the amino acid sequence of SEQ ID NO: 14, and a CGRP-LCDR3 comprising the amino acid sequence of SEQ ID NO: 15;
more preferably,
the CGRP-VH comprises: a CGRP-HCDR1 comprising the amino acid sequence of SEQ ID NO: 22, a CGRP-HCDR2 comprising the amino acid sequence of SEQ ID NO: 106, and a CGRP-HCDR3 comprising the amino acid sequence of SEQ ID NO: 24, and the CGRP-VL comprises: a CGRP-LCDR1 comprising the amino acid sequence of SEQ ID NO: 25, a CGRP-LCDR2 comprising the amino acid sequence of SEQ ID NO: 26, and a CGRP-LCDR3 comprising the amino acid sequence of SEQ ID NO: 27, or
the CGRP-VH comprises: a CGRP-HCDR1 comprising the amino acid sequence of SEQ ID NO: 16, a CGRP-HCDR2 comprising the amino acid sequence of SEQ ID NO: 101, and a CGRP-HCDR3 comprising the amino acid sequence of SEQ ID NO: 18, and the CGRP-VL comprises: a CGRP-LCDR1 comprising the amino acid sequence of SEQ ID NO: 19, a CGRP-LCDR2 comprising the amino acid sequence of SEQ ID NO: 20, and a CGRP-LCDR3 comprising the amino acid sequence of SEQ ID NO: 21.

13. The isolated antibody according to claim 12, wherein:
(i) the CGRP-VH comprises the amino acid sequence of SEQ ID NO: 83, 46, 76, 77, 78, 79, 80, 81, or 82, and the CGRP-VL comprises the amino acid sequence of SEQ ID NO: 85, 47, or 84, or
(ii) the CGRP-VH comprises the amino acid sequence of SEQ ID NO: 72, 44, 68, 69, 70, or 71, and the CGRP-VL comprises the amino acid sequence of SEQ ID NO: 75, 45, 73, or 74, or
(iii) the CGRP-VH comprises the amino acid sequence of SEQ ID NO: 40, 54, 55, or 56, and the CGRP-VL comprises the amino acid sequence of SEQ ID NO: 41, 57, or 58, or
(iv) the CGRP-VH comprises the amino acid sequence of SEQ ID NO: 42, 59, 60, 61, 62, 63, 64, 65, or 66, and the CGRP-VL comprises the amino acid sequence of SEQ ID NO: 43 or 67;
preferably,
(i) the CGRP-VH comprises the amino acid sequence of SEQ ID NO: 83, and the CGRP-VL comprises the amino acid sequence of SEQ ID NO: 85, or
the CGRP-VH comprises the amino acid sequence of SEQ ID NO: 46, and the CGRP-VL comprises the amino acid sequence of SEQ ID NO: 47, or
the CGRP-VH comprises the amino acid sequence of SEQ ID NO: 76, and the CGRP-VL comprises the amino acid sequence of SEQ ID NO: 84, or
the CGRP-VH comprises the amino acid sequence of SEQ ID NO: 77, and the CGRP-VL comprises the amino acid sequence of SEQ ID NO: 84, or
the CGRP-VH comprises the amino acid sequence of SEQ ID NO: 78, and the CGRP-VL comprises the amino acid sequence of SEQ ID NO: 84, or
the CGRP-VH comprises the amino acid sequence of SEQ ID NO: 79, and the CGRP-VL comprises the amino acid sequence of SEQ ID NO: 84, or
the CGRP-VH comprises the amino acid sequence of SEQ ID NO: 80, and the CGRP-VL comprises the amino acid sequence of SEQ ID NO: 84, or
the CGRP-VH comprises the amino acid sequence of SEQ ID NO: 81, and the CGRP-VL comprises the amino acid sequence of SEQ ID NO: 84, or
the CGRP-VH comprises the amino acid sequence of SEQ ID NO: 82, and the CGRP-VL comprises the amino acid sequence of SEQ ID NO: 84; or
(ii) the CGRP-VH comprises the amino acid sequence of SEQ ID NO: 72, and the CGRP-VL comprises the amino acid sequence of SEQ ID NO: 75, or
the CGRP-VH comprises the amino acid sequence of SEQ ID NO: 44, and the CGRP-VL comprises the amino acid sequence of SEQ ID NO: 45, or
the CGRP-VH comprises the amino acid sequence of SEQ ID NO: 68, and the CGRP-VL comprises the amino acid sequence of SEQ ID NO: 73, or
the CGRP-VH comprises the amino acid sequence of SEQ ID NO: 68, and the CGRP-VL comprises the amino acid sequence of SEQ ID NO: 74, or
the CGRP-VH comprises the amino acid sequence of SEQ ID NO: 69, and the CGRP-VL comprises the amino acid sequence of SEQ ID NO: 73, or
the CGRP-VH comprises the amino acid sequence of SEQ ID NO: 69, and the CGRP-VL comprises the amino acid sequence of SEQ ID NO: 74, or
the CGRP-VH comprises the amino acid sequence of SEQ ID NO: 70, and the CGRP-VL comprises the amino acid sequence of SEQ ID NO: 73, or
the CGRP-VH comprises the amino acid sequence of SEQ ID NO: 71, and the CGRP-VL comprises the amino acid sequence of SEQ ID NO: 73, or
the CGRP-VH comprises the amino acid sequence of SEQ ID NO: 71, and the CGRP-VL comprises the amino acid sequence of SEQ ID NO: 74; or
(iii) the CGRP-VH comprises the amino acid sequence of SEQ ID NO: 40, and the CGRP-VL comprises the amino acid sequence of SEQ ID NO: 41, or
the CGRP-VH comprises the amino acid sequence of SEQ ID NO: 54, and the CGRP-VL comprises the amino acid sequence of SEQ ID NO: 57, or
the CGRP-VH comprises the amino acid sequence of SEQ ID NO: 55, and the CGRP-VL comprises the amino acid sequence of SEQ ID NO: 57, or
the CGRP-VH comprises the amino acid sequence of SEQ ID NO: 56, and the CGRP-VL comprises the amino acid sequence of SEQ ID NO: 57, or
the CGRP-VH comprises the amino acid sequence of SEQ ID NO: 54, and the CGRP-VL comprises the amino acid sequence of SEQ ID NO: 58, or
the CGRP-VH comprises the amino acid sequence of SEQ ID NO: 56, and the CGRP-VL comprises the amino acid sequence of SEQ ID NO: 58; or
(iv) the CGRP-VH comprises the amino acid sequence of SEQ ID NO: 42, and the CGRP-VL comprises the amino acid sequence of SEQ ID NO: 43, or
the CGRP-VH comprises the amino acid sequence of SEQ ID NO: 59, and the CGRP-VL comprises the amino acid sequence of SEQ ID NO: 67, or
the CGRP-VH comprises the amino acid sequence of SEQ ID NO: 60, and the CGRP-VL comprises the amino acid sequence of SEQ ID NO: 67, or
the CGRP-VH comprises the amino acid sequence of SEQ ID NO: 61, and the CGRP-VL comprises the amino acid sequence of SEQ ID NO: 67, or
the CGRP-VH comprises the amino acid sequence of SEQ ID NO: 62, and the CGRP-VL comprises the amino acid sequence of SEQ ID NO: 67, or
the CGRP-VH comprises the amino acid sequence of SEQ ID NO: 63, and the CGRP-VL comprises the amino acid sequence of SEQ ID NO: 67, or
the CGRP-VH comprises the amino acid sequence of SEQ ID NO: 64, and the CGRP-VL comprises the amino acid sequence of SEQ ID NO: 67, or
the CGRP-VH comprises the amino acid sequence of SEQ ID NO: 65, and the CGRP-VL comprises the amino acid sequence of SEQ ID NO: 67, or
the CGRP-VH comprises the amino acid sequence of SEQ ID NO: 66, and the CGRP-VL comprises the amino acid sequence of SEQ ID NO: 67;
more preferably,
(i) the CGRP-VH comprises the amino acid sequence of SEQ ID NO: 83, and the CGRP-VL comprises the amino acid sequence of SEQ ID NO: 85, or
the CGRP-VH comprises the amino acid sequence of SEQ ID NO: 81, and the CGRP-VL comprises the amino acid sequence of SEQ ID NO: 84; or
(ii) the CGRP-VH comprises the amino acid sequence of SEQ ID NO: 72, and the CGRP-VL comprises the amino acid sequence of SEQ ID NO: 75.

14. An isolated antibody capable of specifically binding to PACAP, comprising a heavy chain variable region PACAP-VH and a light chain variable region PACAP-VL, wherein:
(v) a PACAP-HCDR1, a PACAP-HCDR2, and a PACAP-HCDR3 in the PACAP-VH comprise the amino acid sequences of a PACAP-HCDR1, a PACAP-HCDR2, and a PACAP-HCDR3 in SEQ ID NO: 87, 48, or 86, respectively, and a PACAP-LCDR1, a PACAP-LCDR2, and a PACAP-LCDR3 in the PACAP-VL comprise the amino acid sequences of a PACAP-LCDR1, a PACAP-LCDR2, and a PACAP-LCDR3 in SEQ ID NO: 90, 49, 88, or 89, respectively, or
(vi) a PACAP-HCDR1, a PACAP-HCDR2, and a PACAP-HCDR3 in the PACAP-VH comprise the amino acid sequences of a PACAP-HCDR1, a PACAP-HCDR2, and a PACAP-HCDR3 in SEQ ID NO: 50 or 91, respectively, and a PACAP-LCDR1, a PACAP-LCDR2, and a PACAP-LCDR3 in the PACAP-VL comprise the amino acid sequences of a PACAP-LCDR1, a PACAP-LCDR2, and a PACAP-LCDR3 in SEQ ID NO: 51 or 92, respectively;
preferably,
(v) the PACAP-VH comprises: a PACAP-HCDR1 comprising the amino acid sequence of SEQ ID NO: 28, a PACAP-HCDR2 comprising the amino acid sequence of SEQ ID NO: 29, and a PACAP-HCDR3 comprising the amino acid sequence of SEQ ID NO: 107 or 30, and the PACAP-VL comprises: a PACAP-LCDR1 comprising the amino acid sequence of SEQ ID NO: 31, a PACAP-LCDR2 comprising the amino acid sequence of SEQ ID NO: 108 or 32, and a PACAP-LCDR3 comprising the amino acid sequence of SEQ ID NO: 33; or
(vi) the PACAP-VH comprises: a PACAP-HCDR1 comprising the amino acid sequence of SEQ ID NO: 34, a PACAP-HCDR2 comprising the amino acid sequence of SEQ ID NO: 35 or 109, and a PACAP-HCDR3 comprising the amino acid sequence of SEQ ID NO: 36, and the PACAP-VL comprises: a PACAP-LCDR1 comprising the amino acid sequence of SEQ ID NO: 37, a PACAP-LCDR2 comprising the amino acid sequence of SEQ ID NO: 38, and a PACAP-LCDR3 comprising the amino acid sequence of SEQ ID NO: 39;
more preferably,
the PACAP-VH comprises: a PACAP-HCDR1 comprising the amino acid sequence of SEQ ID NO: 28, a PACAP-HCDR2 comprising the amino acid sequence of SEQ ID NO: 29, and a PACAP-HCDR3 comprising the amino acid sequence of SEQ ID NO: 107, and the PACAP-VL comprises: a PACAP-LCDR1 comprising the amino acid sequence of SEQ ID NO: 31, a PACAP-LCDR2 comprising the amino acid sequence of SEQ ID NO: 108, and a PACAP-LCDR3 comprising the amino acid sequence of SEQ ID NO: 33.

15. The isolated antibody according to claim 14, wherein:
(v) the PACAP-VH comprises the amino acid sequence of SEQ ID NO: 87, 48, or 86, and the PACAP-VL comprises the amino acid sequence of SEQ ID NO: 90, 49, 88, or 89, or
(vi) the PACAP-VH comprises the amino acid sequence of SEQ ID NO: 50 or 91, and the PACAP-VL comprises the amino acid sequence of SEQ ID NO: 51 or 92;
preferably,
(v) the PACAP-VH comprises the amino acid sequence of SEQ ID NO: 87, and the PACAP-VL comprises the amino acid sequence of SEQ ID NO: 90, or
the PACAP-VH comprises the amino acid sequence of SEQ ID NO: 48, and the PACAP-VL comprises the amino acid sequence of SEQ ID NO: 49, or
the PACAP-VH comprises the amino acid sequence of SEQ ID NO: 86, and the PACAP-VL comprises the amino acid sequence of SEQ ID NO: 88, or
the PACAP-VH comprises the amino acid sequence of SEQ ID NO: 86, and the PACAP-VL comprises the amino acid sequence of SEQ ID NO: 89; or
(vi) the PACAP-VH comprises the amino acid sequence of SEQ ID NO: 50, and the PACAP-VL comprises the amino acid sequence of SEQ ID NO: 51, or
the PACAP-VH comprises the amino acid sequence of SEQ ID NO: 91, and the PACAP-VL comprises the amino acid sequence of SEQ ID NO: 92;
more preferably,
(v) the PACAP-VH comprises the amino acid sequence of SEQ ID NO: 87, and the PACAP-VL comprises the amino acid sequence of SEQ ID NO: 90.

16. The isolated antibody according to claim 12, wherein the antibody is a bispecific antibody;
preferably, the bispecific antibody specifically binds to CGRP and PACAP.

17. A pharmaceutical composition, comprising:
a therapeutically effective amount of the antigen-binding molecule according to claim 1, and
one or more pharmaceutically acceptable vehicles, diluents, buffers, or excipients,
wherein preferably, the pharmaceutical composition further comprises at least one second therapeutic agent.

18. An isolated nucleic acid, wherein the isolated nucleic acid encodes the antigen-binding molecule according to claim 1.

19. A host cell, comprising the isolated nucleic acid according to claim 18.

20. A method for treating a disease, comprising a step of:
administering to a subject a therapeutically effective amount of the antigen-binding molecule according to claim 1,
wherein preferably, the disease is pain;
more preferably, the disease is headache;
most preferably, the disease is migraine or cluster headache.


SEQUENCE LISTING (194 sequences)

SEQ ID NO: 1 - protein, 374 aa, synthetic construct
  CHAIN 1..374: note = PAC1-Fc
MHSDCIFKKEQAMCLEKIQRANELMGFNDSSPGCPGMWDNITCWKPAHVGEMVLVSCPELFRIFNPDQVWETETIGESDFGDSNSLDLSDMGVVSRNCTEDGWSEPFPHYFDACGFDEYESETGDQDYYYLSVKADIEGRMDEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 351 aa, synthetic construct
  CHAIN 1..351: note = VPAC1-Fc
ARLQEECDYVQMIEVQHKQCLEEAQLENETIGCSKMWDNLTCWPATPRGQVVVLACPLIFKLFSSIQGRNVSRSCTDEGWTHLEPGPYPIACGLDDKAASLDEQQTMFYGSVDIEGRMDEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 345 aa, synthetic construct
  CHAIN 1..345: note = VPAC2-Fc
IHPECRFHLEIQEEETKCAELLRSQTEKHKACSGVWDNITCWRPANVGETVTVPCPKVFSNFYSKAGNISKNCTSDGWSETFPDFVDACGYSDPEDESKITFYILVDIEGRMDEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 5 aa, synthetic construct
  DOMAIN 1..5: note = C1 HCDR1
TSWMN

SEQ ID NO: 5 - protein, 17 aa, synthetic construct
  DOMAIN 1..17: note = C1 HCDR2
QIYPGDGYTNYNGKFRG

SEQ ID NO: 6 - protein, 5 aa, synthetic construct
  DOMAIN 1..5: note = C1 HCDR3
DYEAL

SEQ ID NO: 7 - protein, 16 aa, synthetic construct
  DOMAIN 1..16: note = C1 LCDR1
RSSQSLVHSNGNTYFH

SEQ ID NO: 8 - protein, 7 aa, synthetic construct
  DOMAIN 1..7: note = C1 LCDR2
EVSNRFS

SEQ ID NO: 9 - protein, 9 aa, synthetic construct
  DOMAIN 1..9: note = C1 LCDR3
SQSTHVPFT

SEQ ID NO: 10 - protein, 5 aa, synthetic construct
  DOMAIN 1..5: note = C9 HCDR1
DYYMN

SEQ ID NO: 11 - protein, 17 aa, synthetic construct
  DOMAIN 1..17: note = C9 HCDR2
VINPYNGRTTYNRKFKG

SEQ ID NO: 12 - protein, 10 aa, synthetic construct
  DOMAIN 1..10: note = C9 HCDR3
LLIRPHYFDY

SEQ ID NO: 13 - protein, 11 aa, synthetic construct
  DOMAIN 1..11: note = C9 LCDR1
KASQNVGTNVA

SEQ ID NO: 14 - protein, 7 aa, synthetic construct
  DOMAIN 1..7: note = C9 LCDR2
SASYRYS

SEQ ID NO: 15 - protein, 9 aa, synthetic construct
  DOMAIN 1..9: note = C9 LCDR3
QQYNFYPYT

SEQ ID NO: 16 - protein, 5 aa, synthetic construct
  DOMAIN 1..5: note = C21 HCDR1
DYYMN

SEQ ID NO: 17 - protein, 17 aa, synthetic construct
  DOMAIN 1..17: note = C21 HCDR2
DINPNNGGTNYNQKFKG

SEQ ID NO: 18 - protein, 10 aa, synthetic construct
  DOMAIN 1..10: note = C21 HCDR3
IIIVPNYFDY

SEQ ID NO: 19 - protein, 11 aa, synthetic construct
  DOMAIN 1..11: note = C21 LCDR1
KASQNVDTNVA

SEQ ID NO: 20 - protein, 7 aa, synthetic construct
  DOMAIN 1..7: note = C21 LCDR2
SASYRYS

SEQ ID NO: 21 - protein, 9 aa, synthetic construct
  DOMAIN 1..9: note = C21 LCDR3
QQYNNYPYT

SEQ ID NO: 22 - protein, 5 aa, synthetic construct
  DOMAIN 1..5: note = C28 HCDR1
DYYMN

SEQ ID NO: 23 - protein, 17 aa, synthetic construct
  DOMAIN 1..17: note = C28 HCDR2
DINPNNGVTTYNQKFKG

SEQ ID NO: 24 - protein, 10 aa, synthetic construct
  DOMAIN 1..10: note = C28 HCDR3
IIIVPDYFDY

SEQ ID NO: 25 - protein, 11 aa, synthetic construct
  DOMAIN 1..11: note = C28 LCDR1
KASQNVGSNVA

SEQ ID NO: 26 - protein, 7 aa, synthetic construct
  DOMAIN 1..7: note = C28 LCDR2
SASYRYS

SEQ ID NO: 27 - protein, 9 aa, synthetic construct
  DOMAIN 1..9: note = C28 LCDR3
QQYNSYPYT

SEQ ID NO: 28 - protein, 5 aa, synthetic construct
  DOMAIN 1..5: note = P14 HCDR1
NYDMS

SEQ ID NO: 29 - protein, 17 aa, synthetic construct
  DOMAIN 1..17: note = P14 HCDR2
TITGGGSYTWYPDSVKG

SEQ ID NO: 30 - protein, 12 aa, synthetic construct
  DOMAIN 1..12: note = P14 HCDR3
LNGYDQAGAMDY

SEQ ID NO: 31 - protein, 15 aa, synthetic construct
  DOMAIN 1..15: note = P14 LCDR1
KASQSVDYDGDSYMT

SEQ ID NO: 32 - protein, 7 aa, synthetic construct
  DOMAIN 1..7: note = P14 LCDR2
AASNLDS

SEQ ID NO: 33 - protein, 9 aa, synthetic construct
  DOMAIN 1..9: note = P14 LCDR3
QQSNEDPWT

SEQ ID NO: 34 - protein, 5 aa, synthetic construct
  CHAIN 1..5: note = P96 HCDR1
DYYMH

SEQ ID NO: 35 - protein, 17 aa, synthetic construct
  DOMAIN 1..17: note = P96 HCDR2
WIDPENGNTLYDPKFQD

SEQ ID NO: 36 - protein, 12 aa, synthetic construct
  DOMAIN 1..12: note = P96 HCDR3
GVMTTVEGDFDY

SEQ ID NO: 37 - protein, 14 aa, synthetic construct
  DOMAIN 1..14: note = P96 LCDR1
RSSSGTVTTSNYAN

SEQ ID NO: 38 - protein, 7 aa, synthetic construct
  DOMAIN 1..7: note = P96 LCDR2
GTNNRAP

SEQ ID NO: 39 - protein, 9 aa, synthetic construct
  DOMAIN 1..9: note = P96 LCDR3
ALWYSNHWV

SEQ ID NO: 40 - protein, 114 aa, synthetic construct
  DOMAIN 1..114: note = C1#
QVQLQQSGAELVRPGSSVKISCKASGYTFSTSWMNWVKQRPGQGLEWIGQIYPGDGYTNYNGKFRGKATLTADKSSSTAYMQLSSLTSEDSAVYFCAGDYEALWGQGTTLTVSS

SEQ ID NO: 41 - protein, 112 aa, synthetic construct
  DOMAIN 1..112: note = C1#
DVVMTQTPLSLPVSLGDQASISCRSSQSLVHSNGNTYFHWYLQRPGQSPKLLIYEVSNRFSGVPDRFSGSGSGTDFTLKISRVEAEDLGVYFCSQSTHVPFTFGSGTKLEIK

SEQ ID NO: 42 - protein, 119 aa, synthetic construct
  DOMAIN 1..119: note = C9#
EVQLQQSGPVLVKPGASVKMSCKASGYTFTDYYMNWVKQSLGKSLEWIGVINPYNGRTTYNRKFKGKATLTVDKSSSTAYMELNSLTSEDSAVYYCASLLIRPHYFDYWGQGTTLTVSS

SEQ ID NO: 43 - protein, 107 aa, synthetic construct
  DOMAIN 1..107: note = C9#
DIVMTQSQKFMATSVGDRVSVTCKASQNVGTNVAWYQQKPGQSPKALIYSASYRYSVVPDRFTGSGSGTDFTLTIRNVQSEDLAEYHCQQYNFYPYTFGAGTKLELK

SEQ ID NO: 44 - protein, 119 aa, synthetic construct
  DOMAIN 1..119: note = C21#
EVQLQQSGPELVKPGASVRISCKASGYTFTDYYMNWVRQSHRKSLEWIGDINPNNGGTNYNQKFKGKATLTVDKSSSTAYMELRSLTSEDSAVYYCASIIIVPNYFDYWGQGTTLTVSS

SEQ ID NO: 45 - protein, 107 aa, synthetic construct
  DOMAIN 1..107: note = C21#
DIVMTQSQKFMSTSVGDRVSVTCKASQNVDTNVAWFQQRPGQSPKALIYSASYRYSGVPDRFTGSGSGTDFTLTISNVQSEDLADYFCQQYNNYPYTFGGGTKLEIE

SEQ ID NO: 46 - protein, 119 aa, synthetic construct
  DOMAIN 1..119: note = C28#
EVQLQQSGPELVKPGASVKISCEASGYTFTDYYMNWVKQSHGKSLEWIGDINPNNGVTTYNQKFKGKATLTVDKSSSTAYMELRSLTSEDSAVYYCASIIIVPDYFDYWGQGTTLTVSS

SEQ ID NO: 47 - protein, 107 aa, synthetic construct
  DOMAIN 1..107: note = C28#
DIVMTHSQKFMSTSVGDRVSVTCKASQNVGSNVAWYQEKPGQSPKALIYSASYRYSGVPDRFTGSGSGTDFTLTISNVQSEDLAEYFCQQYNSYPYTFGGGTKLEIK

SEQ ID NO: 48 - protein, 121 aa, synthetic construct
  DOMAIN 1..121: note = P14#
DVKLVESGGGLVKPGGSLKVSCAASGFAFNNYDMSWVRQTPEKRLDWVATITGGGSYTWYPDSVKGRFTISRDNARNTLFLQMSSLRSEDTALYYCARLNGYDQAGAMDYWGQGTSVTVSS

SEQ ID NO: 49 - protein, 111 aa, synthetic construct
  DOMAIN 1..111: note = P14#
DIVLTQSPASLAVSLGQRATISCKASQSVDYDGDSYMTWYQQKPGQPPKLLIYAASNLDSGIPARFSGSGSGTDFTLNIHPVEEEDAATYYCQQSNEDPWTFGGGTKLEIK

SEQ ID NO: 50 - protein, 121 aa, synthetic construct
  DOMAIN 1..121: note = P96#
EVQLQQSGADLVRPGALVKLSCKASGFNIKDYYMHWVKQRPEQGLEWIGWIDPENGNTLYDPKFQDKASITTDTSSNTAYLQLSSLTSEDTAVYYCVFGVMTTVEGDFDYWGQGTALTVSS

SEQ ID NO: 51 - protein, 109 aa, synthetic construct
  DOMAIN 1..109: note = P96#
QAVVTQESALTTSPGETVTLTCRSSSGTVTTSNYANWVQEKPDHLFTGLIGGTNNRAPGAPARFSGSLIGDKAALTITGAQTEDEAIYFCALWYSNHWVFGGGTKLTVL

SEQ ID NO: 52 - protein, 330 aa, synthetic construct
  DOMAIN 1..330: note = mol_type = protein organism = synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 53 - protein, 107 aa, synthetic construct [sequence omitted]

SEQ ID NO: 54 - protein, 114 aa, synthetic construct
  DOMAIN 1..114: note = C1H1
EVQLVQSGAEVKKPGSSVKVSCKASGYTFSTSWMNWVRQAPGQGLEWMGQIYPGDGYTNYSGKFRGRVTITADKSTSTAYMELSSLRSEDTAVYYCAGDYEALWGQGTTVTVSS

SEQ ID NO: 55 - protein, 114 aa, synthetic construct
  DOMAIN 1..114: note = C1H2
EVQLVQSGAEVKKPGSSVKVSCKASGYTFSTSWMNWVRQAPGQGLEWMGQIYPGDGYTNYQGKFRGRVTITADKSTSTAYMELSSLRSEDTAVYYCAGDYEALWGQGTTVTVSS

SEQ ID NO: 56 - protein, 114 aa, synthetic construct
  DOMAIN 1..114: note = C1H3
EVQLVQSGAEVKKPGSSVKVSCKASGYTFSTSWMNWVRQAPGQGLEWMGQIYPGDGYTNYNVKFRGRVTITADKSTSTAYMELSSLRSEDTAVYYCAGDYEALWGQGTTVTVSS

SEQ ID NO: 57 - protein, 112 aa, synthetic construct
  DOMAIN 1..112: note = C1L1
DIVMTQTPLSLPVTPGEPASISCRSSQSLVHSNGNTYFHWYLQKPGQSPQLLIYEVSNRFSGVPDRFSGSGSGTDFTLKISRVEAEDVGVYYCSQSTHVPFTFGQGTKLEIK

SEQ ID NO: 58 - protein, 112 aa, synthetic construct
  DOMAIN 1..112: note = C1L2
DIVMTQTPLSLPVTPGEPASISCRSSQSLVHSSGNTYFHWYLQKPGQSPQLLIYEVSNRFSGVPDRFSGSGSGTDFTLKISRVEAEDVGVYYCSQSTHVPFTFGQGTKLEIK

SEQ ID NO: 59 - protein, 119 aa, synthetic construct
  DOMAIN 1..119: note = C9H1
EVQLVQSGAEVKKPGASVKVSCKASGYTFTDYYMNWVRQAPGQRLEWMGVINPYNVRTTYNRKFKGRVTITVDKSASTAYMELSSLRSEDTAVYYCASLLIRPHYFDYWGQGTTVTVSS

SEQ ID NO: 60 - protein, 119 aa, synthetic construct
  DOMAIN 1..119: note = C9H2
EVQLVQSGAEVKKPGASVKVSCKASGYTFTDYYMNWVRQAPGQRLEWIGVINPYNVRTTYNRKFKGRATLTVDKSASTAYMELSSLRSEDTAVYYCASLLIRPHYFDYWGQGTTVTVSS

SEQ ID NO: 61 - protein, 119 aa, synthetic construct
  DOMAIN 1..119: note = C9H3
EVQLVQSGAEVKKPGASVKVSCKASGYTFTDYYMNWVRQAPGQRLEWMGVINPYQGRTTYNRKFKGRVTITVDKSASTAYMELSSLRSEDTAVYYCASLLIRPHYFDYWGQGTTVTVSS

SEQ ID NO: 62 - protein, 119 aa, synthetic construct
  DOMAIN 1..119: note = C9H4
EVQLVQSGAEVKKPGASVKVSCKASGYTFTDYYMNWVRQAPGQRLEWIGVINPYQGRTTYNRKFKGRATLTVDKSASTAYMELSSLRSEDTAVYYCASLLIRPHYFDYWGQGTTVTVSS

SEQ ID NO: 63 - protein, 119 aa, synthetic construct
  DOMAIN 1..119: note = C9H5
EVQLVQSGAEVKKPGASVKVSCKASGYTFTDYYMNWVRQAPGQRLEWMGVINPYSGRTTYNRKFKGRVTITVDKSASTAYMELSSLRSEDTAVYYCASLLIRPHYFDYWGQGTTVTVSS

SEQ ID NO: 64 - protein, 119 aa, synthetic construct
  DOMAIN 1..119: note = C9H6
EVQLVQSGAEVKKPGASVKVSCKASGYTFTDYYMNWVRQAPGQRLEWIGVINPYSGRTTYNRKFKGRATLTVDKSASTAYMELSSLRSEDTAVYYCASLLIRPHYFDYWGQGTTVTVSS

SEQ ID NO: 65 - protein, 119 aa, synthetic construct
  DOMAIN 1..119: note = C9H7
EVQLVQSGAEVKKPGASVKVSCKASGYTFTDYYMNWVRQAPGQRLEWMGVINPYTGRTTYNRKFKGRVTITVDKSASTAYMELSSLRSEDTAVYYCASLLIRPHYFDYWGQGTTVTVSS

SEQ ID NO: 66 - protein, 119 aa, synthetic construct
  DOMAIN 1..119: note = C9H8
EVQLVQSGAEVKKPGASVKVSCKASGYTFTDYYMNWVRQAPGQRLEWIGVINPYTGRTTYNRKFKGRATLTVDKSASTAYMELSSLRSEDTAVYYCASLLIRPHYFDYWGQGTTVTVSS

SEQ ID NO: 67 - protein, 107 aa, synthetic construct
  DOMAIN 1..107: note = C9L1
DIQMTQSPSSLSASVGDRVTITCKASQNVGTNVAWYQQKPGKSPKALIYSASYRYSGVPSRFSGSGSGTDFTLTISSLQPEDVATYHCQQYNFYPYTFGQGTKLEIK

SEQ ID NO: 68 - protein, 119 aa, synthetic construct
  DOMAIN 1..119: note = C21H1
EVQLVQSGAEVKKPGASVKVSCKASGYTFTDYYMNWVRQAPGQRLEWIGDINPNNVGTNYNQKFKGRATLTVDKSASTAYMELSSLRSEDTAVYYCASIIIVPNYFDYWGQGTTVTVSS

SEQ ID NO: 69 - protein, 119 aa, synthetic construct
  DOMAIN 1..119: note = C21H2
EVQLVQSGAEVKKPGASVKVSCKASGYTFTDYYMNWVRQAPGQRLEWIGDINPNQGGTNYNQKFKGRATLTVDKSASTAYMELSSLRSEDTAVYYCASIIIVPNYFDYWGQGTTVTVSS

SEQ ID NO: 70 - protein, 119 aa, synthetic construct
  DOMAIN 1..119: note = C21H3
EVQLVQSGAEVKKPGASVKVSCKASGYTFTDYYMNWVRQAPGQRLEWMGDINPNSGGTNYNQKFKGRVTITVDKSASTAYMELSSLRSEDTAVYYCASIIIVPNYFDYWGQGTTVTVSS

SEQ ID NO: 71 - protein, 119 aa, synthetic construct
  DOMAIN 1..119: note = C21H4
EVQLVQSGAEVKKPGASVKVSCKASGYTFTDYYMNWVRQAPGQRLEWIGDINPNTGGTNYNQKFKGRATLTVDKSASTAYMELSSLRSEDTAVYYCASIIIVPNYFDYWGQGTTVTVSS

SEQ ID NO: 72 - protein, 119 aa, synthetic construct
  DOMAIN 1..119: note = C21H5
EVQLVQSGAEVKKPGASVKVSCKASGYTFTDYYMNWVRQAPGQCLEWIGDINPNQGGTNYNQKFKGRATLTVDKSASTAYMELSSLRSEDTAVYYCASIIIVPNYFDYWGQGTTVTVSS

SEQ ID NO: 73 - protein, 107 aa, synthetic construct
  DOMAIN 1..107: note = C21L1
DIQMTQSPSSLSASVGDRVTITCKASQNVDTNVAWFQQKPGKAPKSLIYSASYRYSGVPSRFSGSGSGTDFTLTISSLQPEDFATYYCQQYNNYPYTFGGGTKVEIK

SEQ ID NO: 74 - protein, 107 aa, synthetic construct
  DOMAIN 1..107: note = C21L2
DIQMTQSPSSLSASVGDRVTITCKASQNVDTNVAWFQQKPGKSPKALIYSASYRYSGVPSRFSGSGSGTDFTLTISSLQPEDFATYYCQQYNNYPYTFGGGTKVEIK

SEQ ID NO: 75 - protein, 107 aa, synthetic construct
  DOMAIN 1..107: note = C21L3
DIQMTQSPSSLSASVGDRVTITCKASQNVDTNVAWFQQKPGKAPKSLIYSASYRYSGVPSRFSGSGSGTDFTLTISSLQPEDFATYYCQQYNNYPYTFGCGTKVEIK

SEQ ID NO: 76 - protein, 119 aa, synthetic construct
  DOMAIN 1..119: note = C28H1
EVQLVQSGAEVKKPGASVKVSCKASGYTFTDYYMNWVRQAPGQRLEWMGDINPNNVVTTYNQKFKGRVTITVDKSASTAYMELSSLRSEDTAVYYCASIIIVPDYFDYWGQGTTVTVSS

SEQ ID NO: 77 - protein, 119 aa, synthetic construct
  DOMAIN 1..119: note = C28H2
EVQLVQSGAEVKKPGASVKVSCKASGYTFTDYYMNWVRQAPGQRLEWIGDINPNNVVTTYNQKFKGRATLTVDKSASTAYMELSSLRSEDTAVYYCASIIIVPDYFDYWGQGTTVTVSS

SEQ ID NO: 78 - protein, 119 aa, synthetic construct
  DOMAIN 1..119: note = C28H3
EVQLVQSGAEVKKPGASVKVSCKASGYTFTDYYMNWVRQAPGQRLEWMGDINPNQGVTTYNQKFKGRVTITVDKSASTAYMELSSLRSEDTAVYYCASIIIVPDYFDYWGQGTTVTVSS

SEQ ID NO: 79 - protein, 119 aa, synthetic construct
  DOMAIN 1..119: note = C28H4
EVQLVQSGAEVKKPGASVKVSCKASGYTFTDYYMNWVRQAPGQRLEWIGDINPNQGVTTYNQKFKGRATLTVDKSASTAYMELSSLRSEDTAVYYCASIIIVPDYFDYWGQGTTVTVSS

SEQ ID NO: 80 - protein, 119 aa, synthetic construct
  DOMAIN 1..119: note = C28H5
EVQLVQSGAEVKKPGASVKVSCKASGYTFTDYYMNWVRQAPGQRLEWIGDINPNSGVTTYNQKFKGRATLTVDKSASTAYMELSSLRSEDTAVYYCASIIIVPDYFDYWGQGTTVTVSS

SEQ ID NO: 81 - protein, 119 aa, synthetic construct
  DOMAIN 1..119: note = C28H6
EVQLVQSGAEVKKPGASVKVSCKASGYTFTDYYMNWVRQAPGQRLEWMGDINPNTGVTTYNQKFKGRVTITVDKSASTAYMELSSLRSEDTAVYYCASIIIVPDYFDYWGQGTTVTVSS

SEQ ID NO: 82 - protein, 119 aa, synthetic construct
  DOMAIN 1..119: note = C28H7
EVQLVQSGAEVKKPGASVKVSCKASGYTFTDYYMNWVRQAPGQRLEWIGDINPNTGVTTYNQKFKGRATLTVDKSASTAYMELSSLRSEDTAVYYCASIIIVPDYFDYWGQGTTVTVSS

SEQ ID NO: 83 - protein, 119 aa, synthetic construct
  DOMAIN 1..119: note = C28H8
EVQLVQSGAEVKKPGASVKVSCKASGYTFTDYYMNWVRQAPGQCLEWMGDINPNTGVTTYNQKFKGRVTITVDKSASTAYMELSSLRSEDTAVYYCASIIIVPDYFDYWGQGTTVTVSS

SEQ ID NO: 84 - protein, 107 aa, synthetic construct
  DOMAIN 1..107: note = C28L1
DIQMTQSPSSVSASVGDRVTITCKASQNVGSNVAWYQQKPGKSPKALIYSASYRYSGVPSRFSGSGSGTDFTLTISSLQPEDFATYYCQQYNSYPYTFGGGTKVEIK

SEQ ID NO: 85 - protein, 107 aa, synthetic construct
  DOMAIN 1..107: note = C28L2
DIQMTQSPSSVSASVGDRVTITCKASQNVGSNVAWYQQKPGKSPKALIYSASYRYSGVPSRFSGSGSGTDFTLTISSLQPEDFATYYCQQYNSYPYTFGCGTKVEIK

SEQ ID NO: 86 - protein, 121 aa, synthetic construct
  DOMAIN 1..121: note = P14H1
EVQLVESGGGLVKPGGSLRLSCAASGFAFNNYDMSWVRQAPGKGLEWVATITGGGSYTWYPDSVKGRFTISRDNAKNSLYLQMNSLRAEDTAVYYCARLTGYDQAGAMDYWGQGTTVTVSS

SEQ ID NO: 87 - protein, 121 aa, synthetic construct
  DOMAIN 1..121: note = P14H2
EVQLVESGGGLVQPGGSLRLSCAASGFAFNNYDMSWVRQAPGKGLEWVATITGGGSYTWYPDSVKGRFTISRDNAKNSLYLQMNSLRAEDTAVYYCARLTGYDQAGAMDYWGQGTTVTVSS

SEQ ID NO: 88 - protein, 111 aa, synthetic construct
  DOMAIN 1..111: note = P14L1
DIVMTQSPDSLAVSLGERATINCKASQSVDYDGDSYMTWYQQKPGQPPKLLIYAASNLDSGVPDRFSGSGSGTDFTLTISSLQAEDVAVYYCQQSNEDPWTFGGGTKVEIK

SEQ ID NO: 89 - protein, 111 aa, synthetic construct
  DOMAIN 1..111: note = P14L2
DIVLTQSPDSLAVSLGERATINCKASQSVDYDGDSYMTWYQQKPGQPPKLLIYAASNLDSGIPDRFSGSGSGTDFTLTISSLQAEDVAVYYCQQSNEDPWTFGGGTKVEIK

SEQ ID NO: 90 - protein, 111 aa, synthetic construct
  DOMAIN 1..111: note = P14L3
DIVLTQSPDSLAVSLGERATINCKASQSVDYDGDSYMTWYQQKPGQPPKLLIYAASHLDSGIPDRFSGSGSGTDFTLTISSLQAEDVAVYYCQQSNEDPWTFGGGTKVEIK

SEQ ID NO: 91 - protein, 121 aa, synthetic construct
  DOMAIN 1..121: note = P96H1
EVQLVQSGAEVKKPGATVKISCKASGFNIKDYYMHWVQQAPGKGLEWMGWIDPENDNTLYDPKFQDRVTITTDTSTNTAYMELSSLRSEDTAVYYCVFGVMTTVEGDFDYWGQGTTVTVSS

SEQ ID NO: 92 - protein, 109 aa, synthetic construct
  DOMAIN 1..109: note = P96L1
ETVVTQEPSLTVSPGGTVTLTCRSSSGTVTTSNYANWVQQKPGQAFRGLIGGTNNRAPGAPARFSGSLLGGKAALTLSGVQPEDEAEYYCALWYSNHWVFGGGTKLTVL

SEQ ID NO: 93 - protein, 17 aa, synthetic construct
  DOMAIN 1..17: note = C1H1 CDR2
QIYPGDGYTNYSGKFRG

SEQ ID NO: 94 - protein, 17 aa, synthetic construct
  DOMAIN 1..17: note = C1H2 CDR2
QIYPGDGYTNYQGKFRG

SEQ ID NO: 95 - protein, 17 aa, synthetic construct
  DOMAIN 1..17: note = C1H3 CDR2
QIYPGDGYTNYNVKFRG

SEQ ID NO: 96 - protein, 17 aa, synthetic construct
  DOMAIN 1..17: note = C9H1 CDR2
VINPYNVRTTYNRKFKG

SEQ ID NO: 97 - protein, 17 aa, synthetic construct
  DOMAIN 1..17: note = C9H3 CDR2
VINPYQGRTTYNRKFKG

SEQ ID NO: 98 - protein, 17 aa, synthetic construct
  DOMAIN 1..17: note = C9H5 CDR2
VINPYSGRTTYNRKFKG

SEQ ID NO: 99 - protein, 17 aa, synthetic construct
  DOMAIN 1..17: note = C9H7 CDR2
VINPYTGRTTYNRKFKG

SEQ ID NO: 100 - protein, 17 aa, synthetic construct
  DOMAIN 1..17: note = C21H1 CDR2
DINPNNVGTNYNQKFKG

SEQ ID NO: 101 - protein, 17 aa, synthetic construct
  DOMAIN 1..17: note = C21H2 CDR2
DINPNQGGTNYNQKFKG

SEQ ID NO: 102 - protein, 17 aa, synthetic construct
  DOMAIN 1..17: note = C21H3 CDR2
DINPNSGGTNYNQKFKG

SEQ ID NO: 103 - protein, 17 aa, synthetic construct
  DOMAIN 1..17: note = C28H1 CDR2
DINPNNVVTTYNQKFKG

SEQ ID NO: 104 - protein, 17 aa, synthetic construct
  DOMAIN 1..17: note = C28H3 CDR2
DINPNQGVTTYNQKFKG

SEQ ID NO: 105 - protein, 17 aa, synthetic construct
  DOMAIN 1..17: note = C28H5 CDR2
DINPNSGVTTYNQKFKG

SEQ ID NO: 106 - protein, 17 aa, synthetic construct
  DOMAIN 1..17: note = C28H6 CDR2
DINPNTGVTTYNQKFKG

SEQ ID NO: 107 - protein, 12 aa, synthetic construct
  DOMAIN 1..12: note = P14H1 CDR3
LTGYDQAGAMDY

SEQ ID NO: 108 - protein, 7 aa, synthetic construct
  DOMAIN 1..7: note = P14L3 CDR2
AASHLDS

SEQ ID NO: 109 - protein, 17 aa, synthetic construct
  DOMAIN 1..17: note = P96H1 CDR2
WIDPENDNTLYDPKFQD

SEQ ID NO: 110 - protein, 227 aa, synthetic construct
  DOMAIN 1..227: note = IgG1(YTE)Fc
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 111 - protein, 695 aa, synthetic construct
  CHAIN 1..695: note = CP-1
EVQLVESGGGLVQPGGSLRLSCAASGFAFNNYDMSWVRQAPGKGLEWVATITGGGSYTWYPDSVKGRFTISRDNAKNSLYLQMNSLRAEDTAVYYCARLTGYDQAGAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCEVQLVQSGAEVKKPGASVKVSCKASGYTFTDYYMNWVRQAPGQCLEWIGDINPNQGGTNYNQKFKGRATLTVDKSASTAYMELSSLRSEDTAVYYCASIIIVPNYFDYWGQGTTVTVSSGGGGSGGGGSGGGGSDIQMTQSPSSLSASVGDRVTITCKASQNVDTNVAWFQQKPGKAPKSLIYSASYRYSGVPSRFSGSGSGTDFTLTISSLQPEDFATYYCQQYNNYPYTFGCGTKVEIKGGGDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 112 - protein, 218 aa, synthetic construct
  CHAIN 1..218: note = CP-1
DIVLTQSPDSLAVSLGERATINCKASQSVDYDGDSYMTWYQQKPGQPPKLLIYAASHLDSGIPDRFSGSGSGTDFTLTISSLQAEDVAVYYCQQSNEDPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 113 - protein, 695 aa, synthetic construct
  CHAIN 1..695: note = CP-2
EVQLVESGGGLVQPGGSLRLSCAASGFAFNNYDMSWVRQAPGKGLEWVATITGGGSYTWYPDSVKGRFTISRDNAKNSLYLQMNSLRAEDTAVYYCARLTGYDQAGAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCEVQLVQSGAEVKKPGASVKVSCKASGYTFTDYYMNWVRQAPGQCLEWMGDINPNTGVTTYNQKFKGRVTITVDKSASTAYMELSSLRSEDTAVYYCASIIIVPDYFDYWGQGTTVTVSSGGGGSGGGGSGGGGSDIQMTQSPSSVSASVGDRVTITCKASQNVGSNVAWYQQKPGKSPKALIYSASYRYSGVPSRFSGSGSGTDFTLTISSLQPEDFATYYCQQYNSYPYTFGCGTKVEIKGGGDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 114 - protein, 218 aa, synthetic construct; identical to SEQ ID NO: 112
  CHAIN 1..218: note = CP-2

SEQ ID NO: 115 - protein, 227 aa, synthetic construct
  DOMAIN 1..227: note = IgG1Fc
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 116 - protein, 227 aa, synthetic construct
  DOMAIN 1..227: note = IgG1Fc
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 117 - protein, 451 aa, synthetic construct
  CHAIN 1..451: note = CP-31
EVQLVESGGGLVQPGGSLRLSCAASGFAFNNYDMSWVRQAPGKGLEWVATITGGGSYTWYPDSVKGRFTISRDNAKNSLYLQMNSLRAEDTAVYYCARLTGYDQAGAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 118 - protein, 218 aa, synthetic construct; identical to SEQ ID NO: 112
  CHAIN 1..218: note = CP-32

SEQ ID NO: 119 - protein, 449 aa, synthetic construct
  CHAIN 1..449: note = CP-33
EVQLVQSGAEVKKPGASVKVSCKASGYTFTDYYMNWVRQAPGQRLEWMGDINPNTGVTTYNQKFKGRVTITVDKSASTAYMELSSLRSEDTAVYYCASIIIVPDYFDYWGQGTTVTVSSGGGGSGIPPKIECLPIDISIDEGKVLTVASAFTGEPTPEVTWSTGGRKIHSQEQGRFHIENTDDSTTLTIKDVQKQDGGLYTLTLRNEFGSDSATVNIHIRSIDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 120 - protein, 209 aa, synthetic construct
  CHAIN 1..209: note = CP-34
DIQMTQSPSSVSASVGDRVTITCKASQNVGSNVAWYQQKPGKSPKALIYSASYRYSGVPSRFSGSGSGTDFTLTISSLQPEDFATYYCQQYNSYPYTFGGGTKVEIKGGGGSSGAPRFLTRPKASVVSVGKDATLSCQIVGNPFPQVSWEKDKQPVTAGVRFRLAQDGDLYRLKILDLQLSDSGQYVCRARNAHGEAFACLGLQVDAEA

SEQ ID NO: 121 - protein, 98 aa, synthetic construct
  DOMAIN 1..98: note = T.0
GIPPKIEALPSDISIDEGKVLTVACAFTGEPTPEVTWSCGGRKIHSQEQGRFHIENTDDLTTLIIMDVQKQDGGLYTLSLGNEFGSDSATVNIHIRSI

SEQ ID NO: 122 - protein, 98 aa, synthetic construct
  DOMAIN 1..98: note = T.1
GIPPKIECLPSDISIDEGKVLTVASAFTGEPTPEVTWSTGGRKIHSQEQGRFHIENTDDLTTLIIMDVQKQDGGLYTLSLGNEFGSDSATVNIHIRSI

SEQ ID NO: 123 - protein, 98 aa, synthetic construct
  DOMAIN 1..98: note = T.2
GIPPKIEALPSDISIDEGKCLTVASAFTGEPTPEVTWSTGGRKIHSQEQGRFHIENTDDLTTLIIMDVQKQDGGLYTLSLGNEFGSDSATVNIHIRSI

SEQ ID NO: 124 - protein, 98 aa, synthetic construct
  DOMAIN 1..98: note = T.3
GIPPKIEALPSDISIDEGKVLTVASCFTGEPTPEVTWSTGGRKIHSQEQGRFHIENTDDLTTLIIMDVQKQDGGLYTLSLGNEFGSDSATVNIHIRSI

SEQ ID NO: 125 - protein, 98 aa, synthetic construct
  DOMAIN 1..98: note = T.4
GIPPKIEALPSDISIDEGKVLCVASAFTGEPTPEVTWSTGGRKIHSQEQGRFHIENTDDLTTLIIMDVQKQDGGLYTLSLGNEFGSDSATVNIHIRSI

SEQ ID NO: 126 - protein, 103 aa, synthetic construct
  DOMAIN 1..103: note = T.5
KAGIRGIPPKIECLPSDISIDEGKVLTVASAFTGEPTPEVTWSTGGRKIHSQEQGRFHIENTDDLTTLIIMDVQKQDGGLYTLSLGNEFGSDSATVNIHIRSI

SEQ ID NO: 127 - protein, 103 aa, synthetic construct
  DOMAIN 1..103: note = T.6
KAGIRGIPPKIEALPSDISIDEGKVLTVACAFTGEPTPEVTWSCGGRKIHSQEQGRFHIENTDDLTTLIIMDVQKQDGGLYTLSLGNEFGSDSATVNIHIRSI

SEQ ID NO: 128 - protein, 98 aa, synthetic construct
  DOMAIN 1..98: note = T.7
GIPPKIECLPSDISIDEGKVLTVASAFTGEPTPEVTWSTGGRKIHSQEQGRFHIENTDDLTTLIISDVQKQDGGLYSLSLGNEFGSDSATVNIHIRSI

SEQ ID NO: 129 - protein, 98 aa, synthetic construct
  DOMAIN 1..98: note = T.8
GIPPKIECLPSDISIDEGKVLTVASAFTGEPTPEVTWSTGGRKIHSQEQGRFHIENTDDLTTLIIKDVQRQDGGLYTLTLRNEFGSDSATVNIHIRSI

SEQ ID NO: 130 - protein, 98 aa, synthetic construct
  DOMAIN 1..98: note = T.9
GIWPKIECLPIDLSIDEGKVLMVASAFTGEPTPEVTWSTGGRKIHSQEQGRFHIENTDDLTTLIIMDVQKQDGGLYTLSLGMEFGSDSATVNIHIRSI

SEQ ID NO: 131 - protein, 98 aa, synthetic construct
  DOMAIN 1..98: note = T.10
GIPPKIECLPIDISIDEGKVLTVASAFTGEPTPEVTWSTGGRKIHSQEQGRFHIENTDDLTTLIIKDVQKQDGGLYTLTLRNEFGSDSATVNIHIRSI

SEQ ID NO: 132 - protein, 98 aa, synthetic construct
  DOMAIN 1..98: note = T.11
GIPPKIECLPSDISIDEGKVLTVASAFTGEPTPEVTWSTSGKKISSEEGGRFHIESTEDLTTLIIMDVQKQDGGVYTLSLGNDLGSDSATVNIHIRSI

SEQ ID NO: 133 - protein, 98 aa, synthetic construct
  DOMAIN 1..98: note = T.12
GIPPKIECLPSDISIDEGKVLTVASAFTGEPTPEVTWSTGGRKIHSEEGGRFHIESTEDLTTLIIMDVQKQDGGVYTLSLGNEFGSDSATVNIHIRSI

SEQ ID NO: 134 - protein, 98 aa, synthetic construct
  DOMAIN 1..98: note = T.13
GIPPKIECLPSDISIDEGKVLTVASAFTGEPTPEVTWSTGGRKIHSQEQGRFHIESTEDLTTLIIMDVQKQDGGVYTLSLGNEFGSDSATVNIHIRSI

SEQ ID NO: 135 - protein, 98 aa, synthetic construct
  DOMAIN 1..98: note = T.14
GIPPKIECLPSDISIDEGKVLTVASAFTGEPTPEVTWSTGGRKIHSQEQGRFHIESTEDLTTLIISDVQKQDGGLYSLSLGNEFGSDSATVNIHIRSI

SEQ ID NO: 136 - protein, 103 aa, synthetic construct
  DOMAIN 1..103: note = T.15
KAGIRGIPPKIECLPSDISIDEGKVLTVASAFTGEPTPEVTWSTGGRKIHSQEQGRFHIESTEDLTTLIISDVQKQDGGLYSLSLGNEFGSDSATVNIHIRSI

SEQ ID NO: 137 - protein, 98 aa, synthetic construct
  DOMAIN 1..98: note = T.16
GIPPKIECLPIDISIDEGKVLTVASAFTGEPTPEVTWSTGGRKIHSQEQGRFHIENTDDSTTLTIKDVQKQDGGLYTLTLRNEFGSDSATVNIHIRSI

SEQ ID NO: 138 - protein, 98 aa, synthetic construct
  DOMAIN 1..98: note = T.17
GIPPKIECLPIDISIDEGKCLTVASAFTGEPTPEVTWSTGGRKIHSQEQGRFHIENTDDSTTLTIKDVQKQDGGLYTLTLRNEFGSDSATVNIHIRSI

SEQ ID NO: 139 - protein, 98 aa, synthetic construct
  DOMAIN 1..98: note = T.18
GIPPKIECLPIDISIDEGKVLTVASCFTGEPTPEVTWSTGGRKIHSQEQGRFHIENTDDSTTLTIKDVQKQDGGLYTLTLRNEFGSDSATVNIHIRSI

SEQ ID NO: 140 - protein, 97 aa, synthetic construct
  DOMAIN 1..97: note = O.0
SGAPRFLTRPKAFVVSVGKDATLSCQIVGNPTPQVSWEKDQQPVAAGARFRLAQDGDLYRLTILDLALGDSGQYVCRARNAIGEAFAAVGLQVDAEA

SEQ ID NO: 141 - protein, 98 aa, synthetic construct
  DOMAIN 1..98: note = OL.0
QGSPPCFLRFPRPVRVVSGAEAELKCVVLGEPPPVVVWEKGGQQLAASERLSFPADGAEHGLLLTAALPTDAGVYVCRARNAAGEAYAAAAVTVLEPP

SEQ ID NO: 142 - protein, 97 aa, synthetic construct
  DOMAIN 1..97: note = O.1
SGAPRFLTRPKAFVVSVGKDATLSCQIVGNPTPQVSWEKDQQPVAAGARFRLAQDGDLYRLTILDLALGDSGQYVCRARNAIGEAFACVGLQVDAEA

SEQ ID NO: 143 - protein, 97 aa, synthetic construct
  DOMAIN 1..97: note = O.2
SGCPRFLTRPKAFVVSVGKDATLSCQIVGNPTPQVSWEKDQQPVAAGARFRLAQDGDLYRLTILDLALGDSGQYVCRARNAIGEAFAAVGLQVDAEA

SEQ ID NO: 144 - protein, 97 aa, synthetic construct
  DOMAIN 1..97: note = O.3
SGAPRFLTCPKAFVVSVGKDATLSCQIVGNPTPQVSWEKDQQPVAAGARFRLAQDGDLYRLTILDLALGDSGQYVCRARNAIGEAFAAVGLQVDAEA

SEQ ID NO: 145 - protein, 97 aa, synthetic construct
  DOMAIN 1..97: note = O.4
SGAPRFLTRPKAFVVSVGKDATLSSQIVGNPTPQVSWEKDQQPVAAGARFRLAQDGDLYRLTILDLALGDSGQYVSRARNAIGEAFACVGLQVDAEA

SEQ ID NO: 146 - protein, 97 aa, synthetic construct
  DOMAIN 1..97: note = O.5
SGCPRFLTRPKAFVVSVGKDATLSSQIVGNPTPQVSWEKDQQPVAAGARFRLAQDGDLYRLTILDLALGDSGQYVSRARNAIGEAFAAVGLQVDAEA

SEQ ID NO: 147 - protein, 97 aa, synthetic construct
  DOMAIN 1..97: note = O.6
SGAPRFLTCPKAFVVSVGKDATLSSQIVGNPTPQVSWEKDQQPVAAGARFRLAQDGDLYRLTILDLALGDSGQYVSRARNAIGEAFAAVGLQVDAEA

SEQ ID NO: 148 - protein, 97 aa, synthetic construct
  DOMAIN 1..97: note = O.7
SGAPRFKTRPKAFVVSVGKDATLSSQIVGNPTPQVSWEKDQQPVAAGARFRLAQDGDLYRLKILDLALGDSGQYVSRARNAIGEAFACVGLQVDAEA

SEQ ID NO: 149 - protein, 97 aa, synthetic construct
  DOMAIN 1..97: note = O.8
SGAPRFKTRPKAFVVSVGKDATLSSQIVGNPTPQVSWEKDQQPVAAGARFRLAQDGDLYRLHILDLALGDSGQYVSRARNAIGEAFACVGLQVDAEA

SEQ ID NO: 150 - protein, 97 aa, synthetic construct
  DOMAIN 1..97: note = O.9
SGAPRFLTRPLAFVVSVGKDATLSSQIVGNPTPQVSWEKDQQPVAAGARFRLAQDGDLYRLKILDLALGDSGQYVSRARNAIGEAFACVGLQVDAEA

SEQ ID NO: 151 - protein, 97 aa, synthetic construct
  DOMAIN 1..97: note = O.10
SGAPRFLTRPLAFVVSVGKDATLSSQIVGNPTPQVSWEKDQQPVAAGARFRLAQDGDLYRLHILDLALGDSGQYVSRARNAIGEAFACVGLQVDAEA

SEQ ID NO: 152 - protein, 102 aa, synthetic construct
  DOMAIN 1..102: note = O.11
DQPQFSGAPRFLTRPKAFVVSVGKDATLSSQIVGNPTPQVSWEKDQQPVAAGARFRLAQDGDLYRLTILDLALGDSGQYVSRARNAIGEAFACVGLQVDAEA

SEQ ID NO: 153 - protein, 102 aa, synthetic construct
  DOMAIN 1..102: note = O.12
DQPQFSGAPRFKTRPKAFVVSVGKDATLSSQIVGNPTPQVSWEKDQQPVAAGARFRLAQDGDLYRLKILDLALGDSGQYVSRARNAIGEAFACVGLQVDAEA

SEQ ID NO: 154 - protein, 102 aa, synthetic construct
  DOMAIN 1..102: note = O.13
DQPQFSGAPRFKTRPKAFVVSVGKDATLSSQIVGNPTPQVSWEKDQQPVAAGARFRLAQDGDLYRLHILDLALGDSGQYVSRARNAIGEAFACVGLQVDAEA

SEQ ID NO: 155 - protein, 102 aa, synthetic construct
  DOMAIN 1..102: note = O.14
DQPQFSGAPRFRTRPKAFVVSVGKDATLSSQIVGNPTPQVSWEKDQQPVAAGARFRLAQDGDLYRLKILDLALGDSGQYVSRARNAIGEAFACVGLQVDAEA

SEQ ID NO: 156 - protein, 102 aa, synthetic construct
  DOMAIN 1..102: note = O.15
DQPQFSGAPRFRTRPKAFVVSVGKDATLSSQIVGNPTPQVSWEKDQQPVAAGARFRLAQDGDLYRLHILDLALGDSGQYVSRARNAIGEAFACVGLQVDAEA

SEQ ID NO: 157 - protein, 102 aa, synthetic construct
  DOMAIN 1..102: note = O.16
DQPQFSGAPRFLTRPKAFVVSVGKDATLSCQIVGNPTPQVSWEKDQQPVAAGARFRLAQDGDLYRLTILDLALGDSGQYVCRARNAIGEAFAAVGLQVDAEA

SEQ ID NO: 158 - protein, 98 aa, synthetic construct
  DOMAIN 1..98: note = OL.1
QGSPPEFLRFPRPVRVVSGAEAELKCVVLGEPPPVVVWEKGGQQLAASERLSFPADGAEHGLLLTAALPTDAGCYVCRARNAAGEAYAAAAVTVLEPP

SEQ ID NO: 159 - protein, 98 aa, synthetic construct
  DOMAIN 1..98: note = OL.2
QGSPPEFLRFPRPVRVVSGAEAELKCVVLGEPPPVVVWEKGGQQLAASERLSFPADGAEHGLLLTAALPTDAGVYVCRARNAACEAYAAAAVTVLEPP

SEQ ID NO: 160 - protein, 98 aa, synthetic construct
  DOMAIN 1..98: note = OL.3
QGSPPEFLRFPRPVRVVSGAEAELKCVVLGEPPPVVVWEKGGQQLAASERLSFPADGAEHGLLLTAALPTDAGVYVCRARNAAGECYAAAAVTVLEPP

SEQ ID NO: 161 - protein, 98 aa, synthetic construct
  DOMAIN 1..98: note = OL.4
QGSPPEFLRFPRPVRVVSGAEAELKSVVLGEPPPVVVWEKGGQQLAASERLSFPADGAEHGLLLTAALPTDAGCYVSRARNAAGEAYAAAAVTVLEPP

SEQ ID NO: 162 - protein, 98 aa, synthetic construct
  DOMAIN 1..98: note = OL.5
QGSPPEFLRFPRPVRVVSGAEAELKSVVLGEPPPVVVWEKGGQQLAASERLSFPADGAEHGLLLTAALPTDAGVYVSRARNAACEAYAAAAVTVLEPP

SEQ ID NO: 163 - protein, 98 aa, synthetic construct
  DOMAIN 1..98: note = OL.6
QGSPPEFLRFPRPVRVVSGAEAELKSVVLGEPPPVVVWEKGGQQLAASERLSFPADGAEHGLLLTAALPTDAGVYVSRARNAAGECYAAAAVTVLEPP

SEQ ID NO: 164 - protein, 97 aa, synthetic construct
  DOMAIN 1..97: note = O.17
SGAPRFLTRPKSYTVSVGKDASLSSQIVGNPTPQVSWEKDQQPVAAGARFRLAQDGDLYRLKILDLALGDSGQYVSRARNAIGEAFACVGLQVDAEA

SEQ ID NO: 165 - protein, 97 aa, synthetic construct
  DOMAIN 1..97: note = O.18
SEAPRFLTRPLAFVVSEGKDATLSSQIVGDPPPEVTWEKDQQPITAGARFRLAQDGDVYRLEILDLQLSDSGQYVSRARNAIGEAFACVGLQVDAEG

SEQ ID NO: 166 - protein, 97 aa, synthetic construct
  DOMAIN 1..97: note = O.19
SGAPRFLTRPLAFVVSVGKLAMLSSQIVGNPTPQVSWEKDQQPVAAGARFRLLQDGDLYRLKILDLALGDSGQYVSRARNAIGEAFACVGLQVDAEA

SEQ ID NO: 167 - protein, 97 aa, synthetic construct
  DOMAIN 1..97: note = O.20
SGAPRFLTRPLAFVVSVGKDATLSSQIVGNPTPQVSWEKDKQPVTAGARFRLAQDGDLYRLKILDLQLSDSGQYVSRARNAIGEAFACLGLQVDAEA

SEQ ID NO: 168 - protein, 97 aa, synthetic construct
  DOMAIN 1..97: note = O.21
SGAPRFLTRPLAFVVSVGKDATLSSQIVGNPTPQVSWEKDQLPVTAGARFRLAQDGDLYRLKILDLTLSDSGQYVSRARNAIGEAFACVGLEVGAEA

SEQ ID NO: 169 - protein, 97 aa, synthetic construct
  DOMAIN 1..97: note = O.22
SGAPRFLTRPLSYVVSVGKDASLSSQIVGNPTPQVSWEKDQLPVTAGARFRLAQDGDLYRLKILDLQLSDSGQYVSRARNAIGEAFACVGLEVGAEA

SEQ ID NO: 170 - protein, 102 aa, synthetic construct
  DOMAIN 1..102: note = O.23
DQPQFSGAPRFLTRPLSYVVSVGKDASLSSQIVGNPTPQVSWEKDQLPVTAGARFRLAQDGDLYRLKILDLQLSDSGQYVSRARNAIGEAFACVGLEVGAEA

SEQ ID NO: 171 - protein, 97 aa, synthetic construct
  DOMAIN 1..97: note = O.24
SGAPRFLTRPKAFVVSVGKDATLSSQIVGNPFPQVSWEKDKQPVTAGVRFRLAQDGDLYRLKILDLQLSDSGQYVSRARNAIGEAFACLGLQVDAEA

SEQ ID NO: 172 - protein, 97 aa, synthetic construct
  DOMAIN 1..97: note = O.25
SGAPRFLTRPKASVVSVGKDATLSSQIVGNPFPQVSWEKDKQPVTAGVRFRLAQDGDLYRLKILDLQLSDSGQYVSRARNAHGEAFACLGLQVDAEA

SEQ ID NO: 173 - protein, 97 aa, synthetic construct
  DOMAIN 1..97: note = O.26
SGCPRFLTRPKASVVSVGKDATLSSQIVGNPFPQVSWEKDKQPVTAGVRFRLAQDGDLYRLKILDLQLSDSGQYVSRARNAHGEAFACLGLQVDAEA

SEQ ID NO: 174 - protein, 97 aa, synthetic construct
  DOMAIN 1..97: note = O.27
SGAPRFLTCPKASVVSVGKDATLSSQIVGNPFPQVSWEKDKQPVTAGVRFRLAQDGDLYRLKILDLQLSDSGQYVSRARNAHGEAFACLGLQVDAEA

SEQ ID NO: 175 - protein, 97 aa, synthetic construct
  DOMAIN 1..97: note = O.28
SGAPRFLTRPKASVVSVGKDATLSCQIVGNPFPQVSWEKDKQPVTAGVRFRLAQDGDLYRLKILDLQLSDSGQYVCRARNAHGEAFACLGLQVDAEA

SEQ ID NO: 176 - protein, 97 aa, synthetic construct
  DOMAIN 1..97: note = O.29
SGCPRFLTRPKASVVSVGKDATLSCQIVGNPFPQVSWEKDKQPVTAGVRFRLAQDGDLYRLKILDLQLSDSGQYVCRARNAHGEAFACLGLQVDAEA

SEQ ID NO: 177 - protein, 97 aa, synthetic construct
  DOMAIN 1..97: note = O.30
SGAPRFLTCPKASVVSVGKDATLSCQIVGNPFPQVSWEKDKQPVTAGVRFRLAQDGDLYRLKILDLQLSDSGQYVCRARNAHGEAFACLGLQVDAEA

SEQ ID NO: 178 - protein, 97 aa, synthetic construct
  DOMAIN 1..97: note = O.31
SGAPRFLTRPKASVVSVGKDATLSSQIVGNPFPQVSWEKDKQPVTAGVRFRLAQDGDLYRLKILDCQLSDSGQYVSRARNAHGEAFACLGLQCDAEA

SEQ ID NO: 179 - protein, 97 aa, synthetic construct
  DOMAIN 1..97: note = O.32
SGCPRFLTRPKASVVSVGKDATLSSQIVGNPFPQVSWEKDKQPVTAGVRFRLAQDGDLYRLKILDCQLSDSGQYVSRARNAHGEAFACLGLQCDAEA

SEQ ID NO: 180 - protein, 97 aa, synthetic construct
  DOMAIN 1..97: note = O.33
SGAPRFLTCPKASVVSVGKDATLSSQIVGNPFPQVSWEKDKQPVTAGVRFRLAQDGDLYRLKILDCQLSDSGQYVSRARNAHGEAFACLGLQCDAEA

SEQ ID NO: 181 - protein, 227 aa, synthetic construct
  DOMAIN 1..227: note = Fc1
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 182 - protein, 227 aa, synthetic construct
  DOMAIN 1..227: note = Fc2
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 183 - protein, 103 aa, synthetic construct
  DOMAIN 1..103: note = CH1
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 184 - protein, 118 aa, synthetic construct
  DOMAIN 1..118: note = VH2-L
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSS

SEQ ID NO: 185 - protein, 108 aa, synthetic construct
  DOMAIN 1..108: note = VL2-L
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIK

SEQ ID NO: 186 - skipped

SEQ ID NO: 187 - protein, 5 aa, synthetic construct
  DOMAIN 1..5: note = linker
GGGGS

SEQ ID NO: 188 - protein, 10 aa, synthetic construct
  DOMAIN 1..10: note = linker
GGGGSGGGGS

SEQ ID NO: 189 - protein, 15 aa, synthetic construct
  DOMAIN 1..15: note = linker
GGGGSGGGGSGGGGS

SEQ ID NO: 190 - protein, 5 aa, synthetic construct
  DOMAIN 1..5: note = linker
ASTKG

SEQ ID NO: 191 - protein, 5 aa, synthetic construct
  DOMAIN 1..5: note = linker
RTVAS

SEQ ID NO: 192 - protein, 17 aa, synthetic construct
DINPNTGGTNYNQKFKG

SEQ ID NO: 193 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 194 - protein, 695 aa, synthetic construct
EVQLVESGGGLVQPGGSLRLSCAASGFAFNNYDMSWVRQAPGKGLEWVATITGGGSYTWYPDSVKGRFTISRDNAKNSLYLQMNSLRAEDTAVYYCARLTGYDQAGAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCEVQLVQSGAEVKKPGASVKVSCKASGYTFTDYYMNWVRQAPGQCLEWMGDINPNTGVTTYNQKFKGRVTITVDKSASTAYMELSSLRSEDTAVYYCASIIIVPDYFDYWGQGTTVTVSSGGGGSGGGGSGGGGSDIQMTQSPSSVSASVGDRVTITCKASQNVGSNVAWYQQKPGKSPKALIYSASYRYSGVPSRFSGSGSGTDFTLTISSLQPEDFATYYCQQYNSYPYTFGCGTKVEIKGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK